(19) United States
(12) Patent Application Publication
(10) Pub. No.: US 2013/0060033 A1
(43) Pub. Date: Mar. 7, 2013

(54) LIGHT-EMITTING ELEMENT, LIGHT-EMITTING DEVICE, ELECTRONIC DEVICE, LIGHTING DEVICE, AND HETEROCYCLIC COMPOUND

(71) Applicants: Satoshi SEO, Sagamihara (JP); Satoko SHITAGAKI, Isehara (JP); Hiroki SUZUKI, Atsugi (JP); Kaori OGITA, Isehara (JP); Tomohiro KUBOTA, Isehara (JP); Miyako MORIKUBO, Atsugi (JP); Yasushi KITANO, Atsugi (JP)

(72) Inventors: Satoshi SEO, Sagamihara (JP); Satoko SHITAGAKI, Isehara (JP); Hiroki SUZUKI, Atsugi (JP); Kaori OGITA, Isehara (JP); Tomohiro KUBOTA, Isehara (JP); Miyako MORIKUBO, Atsugi (JP); Yasushi KITANO, Atsugi (JP)

(73) Assignee: SEMICONDUCTOR ENERGY LABORATORY CO., LTD., Atsugi-shi (JP)

(21) Appl. No.: 13/599,191

(22) Filed: Aug. 30, 2012

(30) Foreign Application Priority Data
Aug. 31, 2011 (JP) 2011-188235
Jun. 27, 2012 (JP) 2012-144180

(51) Int. Cl. C07D 241/38

(52) U.S. Cl. 544/343

(57) ABSTRACT

Provided is a novel heterocyclic compound which has excellent heat resistance and can be used as a host material for a light-emitting substance (a substance emitting fluorescence or a substance emitting phosphorescence). A light-emitting element includes a heterocyclic compound which includes one dibenzo[f,h]quinoxaline ring, one ring having a hole-transport skeleton, and two to eight benzene rings. Note that in the above structure, the molecular weight of the heterocyclic compound is greater than or equal to 564 and less than or equal to 1000.

### TECHNICAL FIELD

The present invention relates to a light-emitting element, a light-emitting device, an electronic device, a lighting device, and a heterocyclic compound.

### BACKGROUND ART

In recent years, research and development have been extensively conducted on light-emitting elements utilizing electroluminescence (EL). In the basic structure of such a light-emitting element, a layer containing a light-emitting substance is interposed between a pair of electrodes. By voltage application to this element, light emission from the light-emitting substance can be obtained.

Such light-emitting elements are self-luminous elements and have advantages over liquid crystal displays in having high pixel visibility and eliminating the need for backlights, for example; thus, light-emitting elements are thought to be suitable for flat panel display elements. Light-emitting elements are also highly advantageous in that they can be thin and lightweight. Furthermore, very high speed response is one of the features of such elements.

Furthermore, since such light-emitting elements can be formed in a film form, they make it possible to provide planar light emission easily. Accordingly, large-area elements with the use of planar light emission can be easily formed. This is a feature difficult to obtain with point light sources typified by incandescent lamps and LEDs or linear light sources typified by fluorescent lamps. Thus, the light-emitting elements also have great potential as planar light sources applicable to lightings and the like.

Such light-emitting elements utilizing electroluminescence can be broadly classified according to whether a light-emitting substance is an organic compound or an inorganic compound. In the case of an organic EL element in which a layer containing an organic compound used as a light-emitting substance is provided between a pair of electrodes, application of a voltage to the light-emitting element causes injection of electrons from a cathode and holes from an anode into the layer containing the organic compound having a light-emitting property and thus a current flows. The injected electrons and holes then lead the organic compound having a light-emitting property to its excited state, whereby light emission is obtained from the excited organic compound having a light-emitting property.

The excited state formed by an organic compound can be a singlet excited state or a triplet excited state. Emission from the singlet excited state (S*) is called fluorescence, and emission from the triplet excited state (T*) is called phosphorescence. In addition, the statistical generation ratio thereof in a light-emitting element is considered to be as follows: S*:T*=1:3.

In a compound which converts energy of a singlet excited state into light emission (hereinafter, referred to as a fluorescent compound), at room temperature, light emission from the triplet excited state (phosphorescence) is not observed while only light emission from the singlet excited state (fluorescence) is observed. Therefore, the internal quantum efficiency (the ratio of generated photons to injected carriers) of a light-emitting element using a fluorescent compound is assumed to have a theoretical limit of 25% based on the ratio of S* to T* which is 1:3.

In contrast, in a compound which converts energy of a triplet excited state into light emission (hereinafter, referred to as a phosphorescent compound), light emission from the triplet excited state (phosphorescence) is observed. Further, in a phosphorescent compound, since intersystem crossing (i.e. transfer from a singlet excited state to a triplet excited state) easily occurs, the internal quantum efficiency can be increased to 75% to 100% in theory. In other words, the emission efficiency can be three to four times as much as that of a fluorescent compound. For this reason, light-emitting elements using phosphorescent compounds are now under active development in order to realize highly efficient light-emitting elements.

When a light-emitting layer of a light-emitting element is formed using a phosphorescent compound described above, in order to suppress concentration quenching or quenching due to triplet-triplet annihilation in the phosphorescent compound, the light-emitting layer is often formed such that the phosphorescent compound is dispersed in a matrix of another compound. Here, the compound serving as the matrix is called a host material, and the compound dispersed in the matrix, such as the phosphorescent compound, is called a guest material.

In the case where a phosphorescent compound is a guest material, a host material needs to have higher triplet excitation energy (a larger energy difference between a ground state and a triplet excited state) than the phosphorescent compound.

Furthermore, since singlet excitation energy (an energy difference between a ground state and a singlet excited state) is higher than triplet excitation energy, a substance that has high triplet excitation energy also has high singlet excitation energy. Therefore, the above substance that has high triplet excitation energy is also effective in a light-emitting element using a fluorescent compound as a light-emitting substance.

Studies have been conducted on compounds having dibenzo[f,h]quinoxaline rings, which are examples of the host material used when a phosphorescent compound is a guest material (e.g., see Patent Documents 1 and 2).

### REFERENCES

### Patent Document

[Patent Document 1] PCT International Publication No. 03/058667
[Patent Document 2] Japanese Published Patent Application No. 2007-189001

### DISCLOSURE OF INVENTION

According to an embodiment of the present invention, a novel heterocyclic compound which has excellent heat resistance and can be used as a host material for a light-emitting substance (a substance emitting fluorescence or a substance emitting phosphorescence) is provided. Further, according to an embodiment of the present invention, a light-emitting element which has excellent heat resistance, a low driving voltage, high current efficiency, and a long lifetime is provided. Furthermore, according to an embodiment of the present invention, a light-emitting device, an electronic device, and a lighting device in which power consumption is reduced with the use of the light-emitting element are provided.

An embodiment of the present invention is a light-emitting element including a heterocyclic compound which includes one dibenzo[f,h]quinoxaline ring, one ring having a hole-transport skeleton, and two to eight benzene rings.

Further, an embodiment of the present invention is a light-emitting element including a heterocyclic compound which includes one dibenzo[f,h]quinoxaline ring and one ring having a hole-transport skeleton and whose molecular weight is greater than or equal to 564 and less than or equal to 1000. With the use of the heterocyclic compound of an embodiment of the present invention, a uniform film can be formed by vacuum evaporation; however, if the molecular weight is too large, the evaporation temperature is increased, which leads to a problem of thermal decomposition or the like. Therefore, the molecular weight is preferably in the above range. Further, the heterocyclic compound preferably includes one dibenzo[f,h]quinoxaline ring, one ring having a hole-transport skeleton, and two to eight benzene rings.

Another embodiment of the present invention is a light-emitting element including a heterocyclic compound which includes one dibenzo[f,h]quinoxaline ring, one ring having a hole-transport skeleton, and four to eight benzene rings.

Further, an embodiment of the present invention is a light-emitting element including a heterocyclic compound which includes one dibenzo[f,h]quinoxaline ring and one ring having a hole-transport skeleton and whose molecular weight is greater than or equal to 716 and less than or equal to 1000. With the use of the heterocyclic compound of an embodiment of the present invention, a uniform film can be formed by vacuum evaporation; however, if the molecular weight is too large, the evaporation temperature is increased, which leads to a problem of thermal decomposition or the like. Therefore, the molecular weight is preferably in the above range. Further, the heterocyclic compound preferably includes one dibenzo[f,h]quinoxaline ring, one ring having a hole-transport skeleton, and four to eight benzene rings.

In each of the structures, the ring having a hole-transport skeleton is a carbazole ring, a dibenzothiophene ring, or a dibenzofuran ring.

Further, in each of the structures, the heterocyclic compound has a biphenyl group or a biphenyldiyl group. With a biphenyl skeleton, heat resistance can be improved. Moreover, a biphenyl skeleton forms a bulky skeleton to prevent crystallization. In view of the above, an m-biphenyl group or a 3,3′-biphenyldiyl group is particularly preferable.

Another embodiment of the present invention is a heterocyclic compound represented by the following general formula (G1).

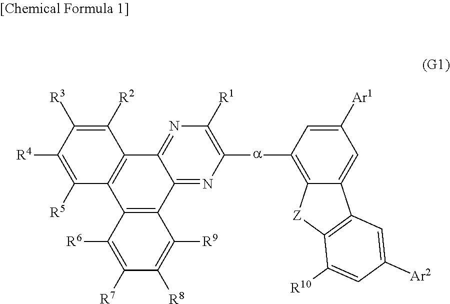

Note that in the formula, α represents a substituted or unsubstituted phenylene group, Ar¹ and Ar² each represent a substituted or unsubstituted biphenyl group, R¹ to R¹⁰ independently represent hydrogen, an alkyl group having 1 to 4 carbon atoms, or a substituted or unsubstituted aryl group having 6 to 13 carbon atoms, and Z represents oxygen or sulfur.

In the above structure, the phenylene group represented by α is an m-phenylene group.

Another embodiment of the present invention is a heterocyclic compound represented by the following general formula (G2).

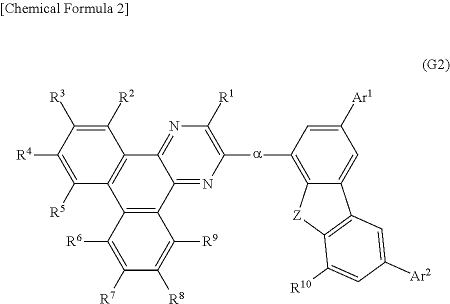

Note that in the formula, α represents a substituted or unsubstituted biphenyldiyl group, Ar¹ and Ar² each represent a substituted or unsubstituted phenyl group or a substituted or unsubstituted biphenyl group, R¹ to R¹⁰ independently represent hydrogen, an alkyl group having 1 to 4 carbon atoms, or a substituted or unsubstituted aryl group having 6 to 13 carbon atoms, and Z represents oxygen or sulfur.

In the above structure, the biphenyldiyl group represented by α is a biphenyl-3,3′-diyl group.

Another embodiment of the present invention is a heterocyclic compound represented by the following structural formula (418).

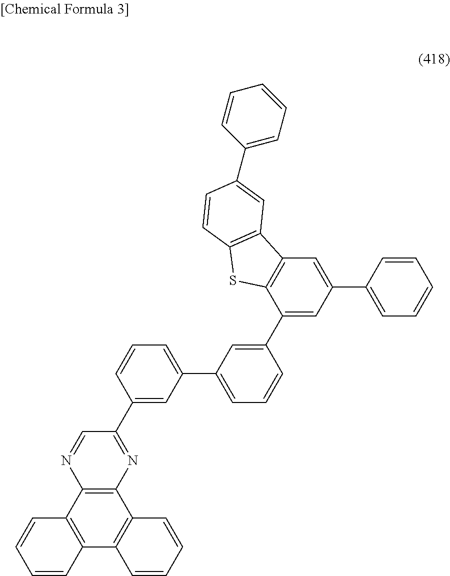 [SMILES: c1ccc(-c2ccc3sc4c(-c5cccc(-c6cccc(-c7cnc8c9ccccc9c9ccccc9c8n7)c6)c5)cc(-c5ccccc5)cc4c3c2)cc1]

Another embodiment of the present invention is a heterocyclic compound represented by the following structural formula (400).

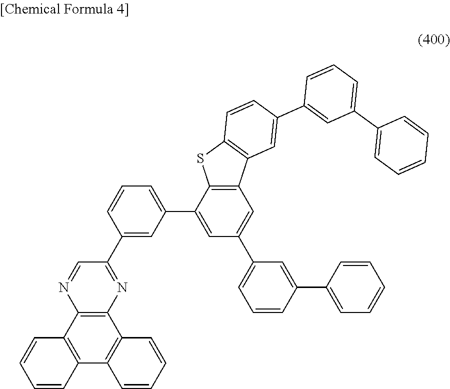 [SMILES: c1ccc(-c2cccc(-c3ccc4sc5c(-c6cccc(-c7cnc8c9ccccc9c9ccccc9c8n7)c6)cc(-c6cccc(-c7ccccc7)c6)cc5c4c3)c2)cc1]

The heterocyclic compound according to any one of embodiments of the present invention has a hole-transport skeleton in addition to a dibenzo[f,h]quinoxaline ring, thereby easily accepting holes. Further, the heterocyclic compound according to any one of embodiments of the present invention includes a plurality of benzene rings, thereby having excellent heat resistance. Accordingly, by use of the heterocyclic compound according to any one of embodiments of the present invention as a host material of a light-emitting layer, a light-emitting element can have excellent heat resistance, and electrons and holes recombine in the light-emitting layer, so that it is possible to suppress the decrease in the lifetime of the light-emitting element. Furthermore, the introduction of a hole-transport skeleton enables the heterocyclic compound according to any one of embodiments of the present invention to have a three-dimensionally bulky structure, and the heterocyclic compound is difficult to crystallize when formed into a film. By the use of the heterocyclic compound for a light-emitting element, the element can have a long lifetime. Moreover, in this heterocyclic compound, in the case where a dibenzo[f,h]quinoxaline ring and a hole-transport skeleton are bonded through a benzene ring, decreases in band gap and triplet excitation energy can be prevented as compared with a compound in which a dibenzo[f,h]quinoxaline ring and a hole-transport skeleton are directly bonded. By the use of the heterocyclic compound for a light-emitting element, the element can have high current efficiency. Accordingly, a light-emitting element including the heterocyclic compound according to any one of embodiments of the present invention is also an embodiment of the present invention.

That is, another embodiment of the present invention is a light-emitting element which includes an EL layer between a pair of electrodes, where the heterocyclic compound according to any one of embodiments of the present invention is included in a light-emitting layer of the EL layer.

Other embodiments of the present invention are not only a light-emitting device including the light-emitting element but also an electronic device and a lighting device each including the light-emitting device. Accordingly, a light-emitting device in this specification refers to an image display device or a light source (including an illuminating device). Further, the light-emitting device includes any of the following modules in its category: a module in which a connector such as a flexible printed circuit (FPC), a tape automated bonding (TAB) tape, or a tape carrier package (TCP) is attached to a light-emitting device; a module having a TAB tape or a TCP provided with a printed wiring board at the end thereof; and a module having an integrated circuit (IC) directly mounted over a light-emitting element by a chip on glass (COG) method.

According to an embodiment of the present invention, a novel heterocyclic compound which has excellent heat resistance and can be used as a host material for a light-emitting substance (a substance emitting fluorescence or a substance emitting phosphorescence) can be provided. Further, according to an embodiment of the present invention, a light-emitting element which has excellent heat resistance, a low driving voltage, high current efficiency, and a long lifetime can be provided. Furthermore, according to an embodiment of the present invention, a light-emitting device, an electronic device, and a lighting device in which power consumption is reduced with the use of the light-emitting element can be provided.

### BRIEF DESCRIPTION OF DRAWINGS

In the accompanying drawings:

FIG. 1 illustrates a structure of a light-emitting element;

FIG. 2 illustrates a structure of a light-emitting element;

FIGS. 3A and 3B each illustrate a structure of a light-emitting element;

FIG. 4 illustrates a light-emitting device;

FIGS. 5A and 5B illustrate a light-emitting device;

FIGS. 6A to 6D each illustrate an electronic device;

FIGS. 7A to 7C illustrate an electronic device;

FIG. 8 illustrates lighting devices;

FIG. 9 shows a ¹H-NMR chart of a heterocyclic compound represented by a structural formula (418);

FIG. 10 shows a ¹H-NMR chart of a heterocyclic compound represented by a structural formula (400);

FIG. 11 illustrates a light-emitting element;

FIG. 12 shows luminance versus current density characteristics of a light-emitting element 1;

FIG. 13 shows luminance versus voltage characteristics of a light-emitting element 1;

FIG. 14 shows current efficiency versus luminance characteristics of a light-emitting element 1;

FIG. 15 shows an emission spectrum of a light-emitting element 1;

FIG. 16 shows results of preservation tests at 80° C. of a light-emitting element 2 and a comparative light-emitting element;

FIG. 17 shows results of preservation tests at 80° C. of a light-emitting element 3 and a comparative light-emitting element;

FIG. 18 shows results of preservation tests at 80° C.;

FIG. 19 shows an LC-MS measurement result of a heterocyclic compound represented by a structural formula (418);

FIG. 20 shows an LC-MS measurement result of a heterocyclic compound represented by a structural formula (400);

FIG. 21 shows an LC-MS measurement result of a heterocyclic compound represented by a structural formula (103);

FIGS. 22A and 22B each show an LC-MS measurement result of a heterocyclic compound represented by a structural formula (113);

FIG. 23 shows luminance versus current density characteristics of a light-emitting element 4;

FIG. 24 shows luminance versus voltage characteristics of a light-emitting element 4;

FIG. 25 shows current efficiency versus luminance characteristics of a light-emitting element 4;

FIG. 26 shows an emission spectrum of a light-emitting element 4;

FIG. 27 shows a result of a preservation test at 100° C. of a light-emitting element 4;

FIG. 28 shows a result of a preservation test at 100° C.;

FIGS. 29A and 29B show a ¹H-NMR chart of a heterocyclic compound represented by a structural formula (131);

FIGS. 30A and 30B each show an LC-MS measurement result of a heterocyclic compound represented by a structural formula (131);

FIG. 31 shows a result of TOF-SIMS (positive ion) measurement of a heterocyclic compound represented by a structural formula (400);

FIG. 32 shows a result of TOF-SIMS (negative ion) measurement of a heterocyclic compound represented by a structural formula (103);

FIG. 33 shows a result of TOF-SIMS (positive ion) measurement of a heterocyclic compound represented by a structural formula (113);

FIGS. 34A and 34B show a ¹H-NMR chart of a heterocyclic compound represented by a structural formula (203); and

FIG. 35 shows an LC-MS measurement result of a heterocyclic compound represented by a structural formula (203).

### BEST MODE FOR CARRYING OUT THE INVENTION

Hereinafter, embodiments and examples of the present invention will be described in detail with reference to the drawings. Note that the present invention is not limited to the description given below, and various changes and modifications can be made without departing from the spirit and scope of the present invention. Therefore, the present invention should not be interpreted as being limited to the description of the embodiments and examples given below.

### Embodiment 1

In this embodiment, a light-emitting element which is an embodiment of the present invention will be described.

A light-emitting element according to an embodiment of the present invention includes a heterocyclic compound which includes one dibenzo[f,h]quinoxaline ring, one ring having a hole-transport skeleton, and two to eight benzene rings. The number of the benzene rings is preferably two to eight because heat resistance of the compound can be increased. In terms of heat resistance, the light-emitting element according to an embodiment of the present invention preferably includes a heterocyclic compound which includes one dibenzo[f,h]quinoxaline ring and one ring having a hole-transport skeleton and whose molecular weight is greater than or equal to 564 and less than or equal to 1000. Note that such an effect is particularly obtained in the case where the heterocyclic compound is used for a light-emitting layer of a light-emitting element.

In order to further increase heat resistance of a compound, a light-emitting element according to an embodiment of the present invention may include a heterocyclic compound which includes one dibenzo[f,h]quinoxaline ring, one ring having a hole-transport skeleton, and four to eight benzene rings. In terms of heat resistance, the light-emitting element according to an embodiment of the present invention preferably includes a heterocyclic compound which includes one dibenzo[f,h]quinoxaline ring and one ring having a hole-transport skeleton and whose molecular weight is greater than or equal to 716 and less than or equal to 1000.

The heterocyclic compound included in either of the light-emitting elements has a biphenyl group or a biphenyldiyl group. As the ring having a hole-transport skeleton, a carbazole ring, dibenzothiophene ring, or a dibenzofuran ring is used.

An embodiment of the present invention is a heterocyclic compound represented by a general formula (G1).

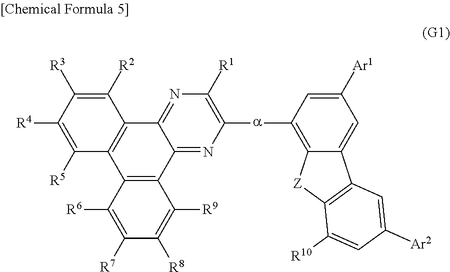

In the general formula (G1), α represents a substituted or unsubstituted phenylene group, Ar¹ and Ar² each represent a substituted or unsubstituted biphenyl group, R¹ to R¹⁰ independently represent hydrogen, an alkyl group having 1 to 4 carbon atoms, or a substituted or unsubstituted aryl group having 6 to 13 carbon atoms, and Z represents oxygen or sulfur.

An embodiment of the present invention is a heterocyclic compound represented by a general formula (G2).

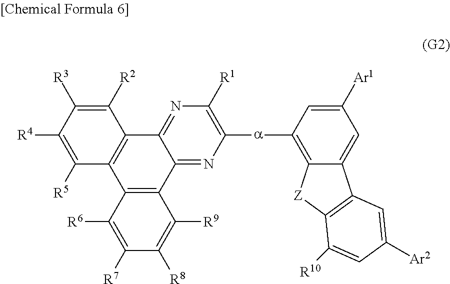

In the general formula (G2), α represents a substituted or unsubstituted biphenyldiyl group, Ar¹ and Ar² each represent a substituted or unsubstituted phenyl group or a substituted or unsubstituted biphenyl group, R¹ to R¹⁰ independently represent hydrogen, an alkyl group having 1 to 4 carbon atoms, or a substituted or unsubstituted aryl group having 6 to 13 carbon atoms, and Z represents oxygen or sulfur.

As specific structures of a in the general formula (G1) or (G2), there are substituents represented by structural formulae (1-1) to (1-9), for example.

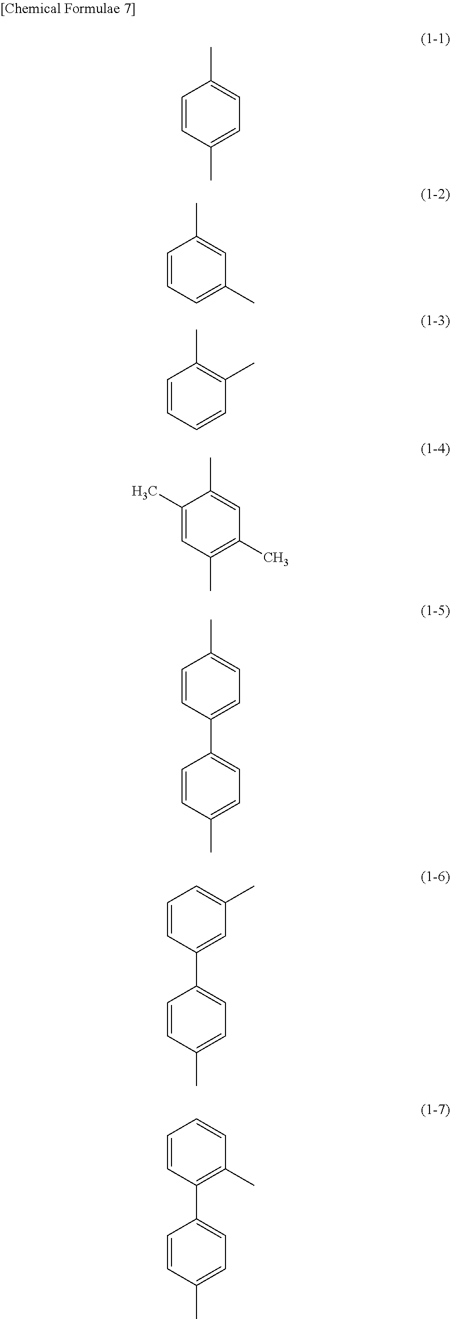

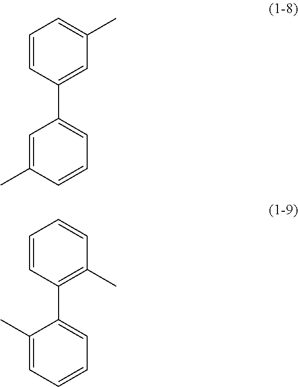

As specific structures of Ar¹ and Ar² in the general formula (G1) or (G2), there are substituents represented by structural formulae (2-10) to (2-16), for example.

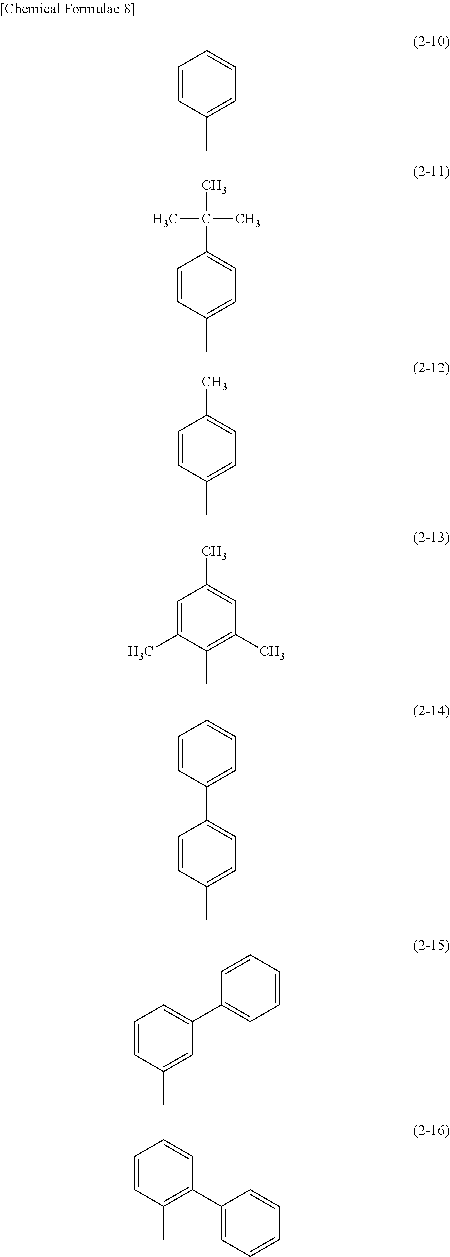

As specific structures of R¹ to R¹⁰ in the general formula (G1) or (G2), there are substituents represented by structural formulae (2-1) to (2-19), for example.

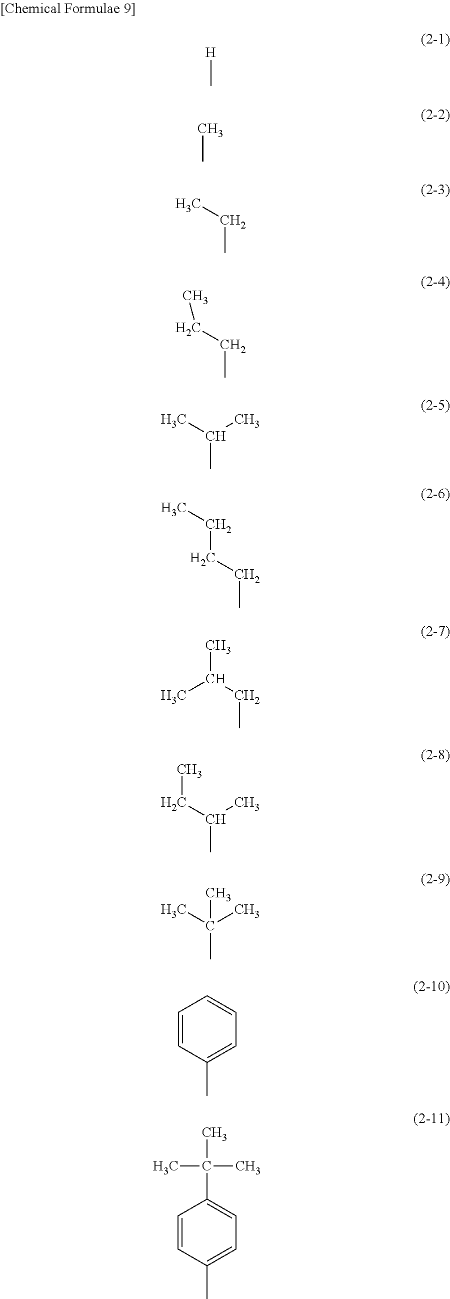

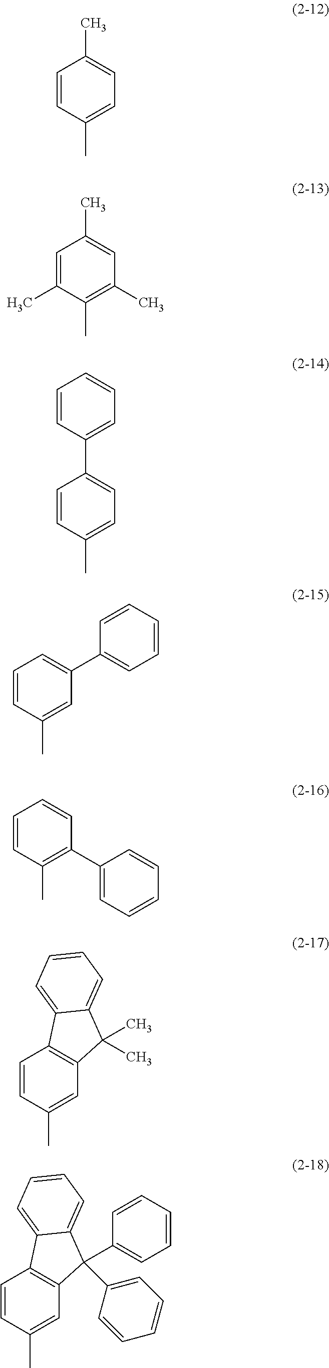

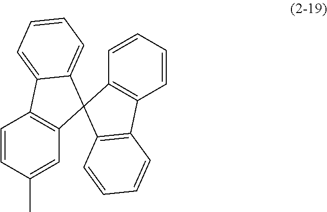

Specific examples of a heterocyclic compound which can be used in an embodiment of the present invention include heterocyclic compounds represented by structural formulae (100) to (131), (200) to (230), (300) to (329), (400) to (436), and (500) to (536). Note that specific examples of the compound represented by the general formula (G1) include the compounds represented by the structural formulae (400) to (417) and (500) to (517). Further, specific examples of the compound represented by the general formula (G2) include the compounds represented by the structural formulae (418) to (436) and (518) to (536). Note that the present invention is not limited to the above compounds.

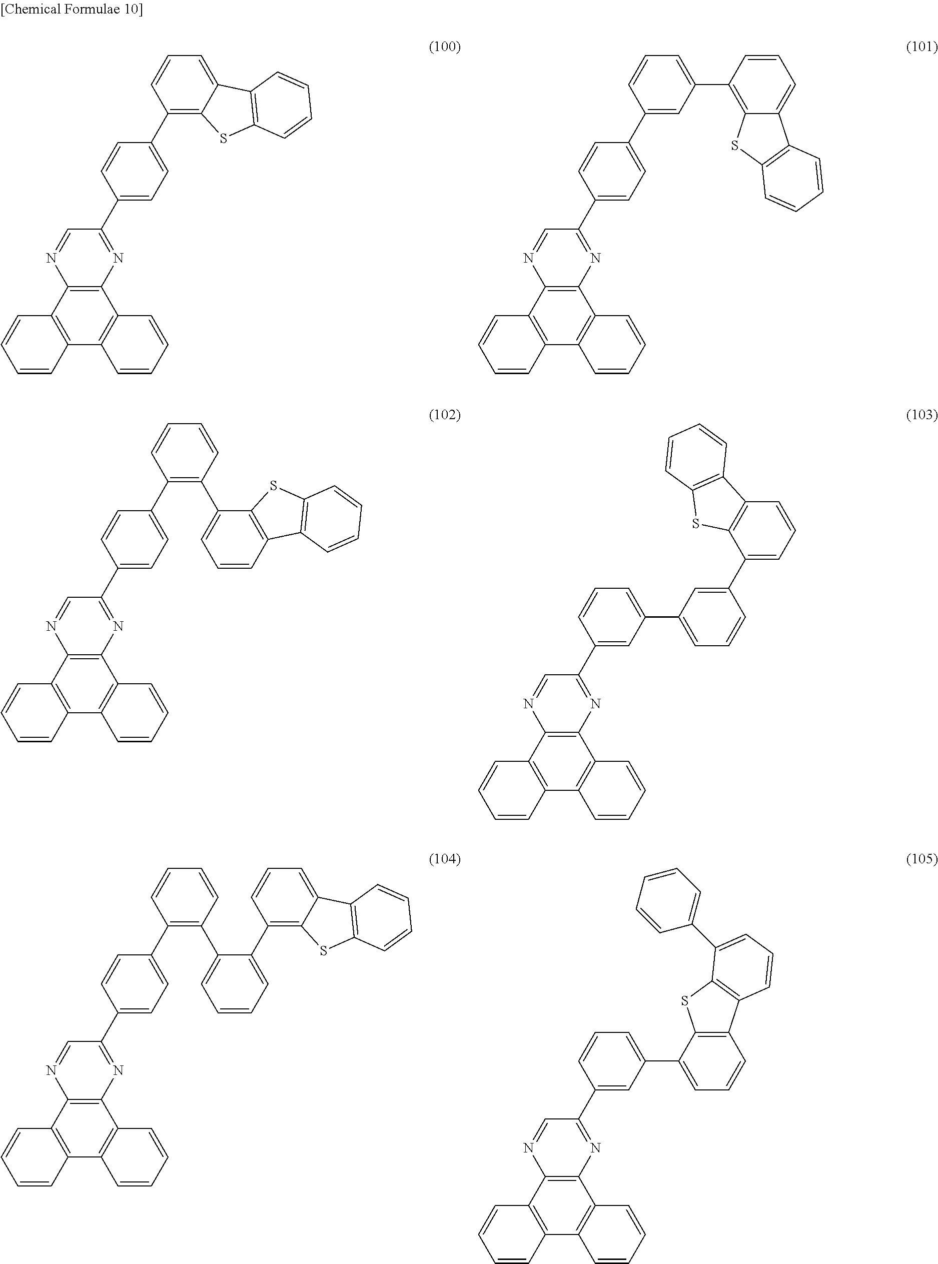

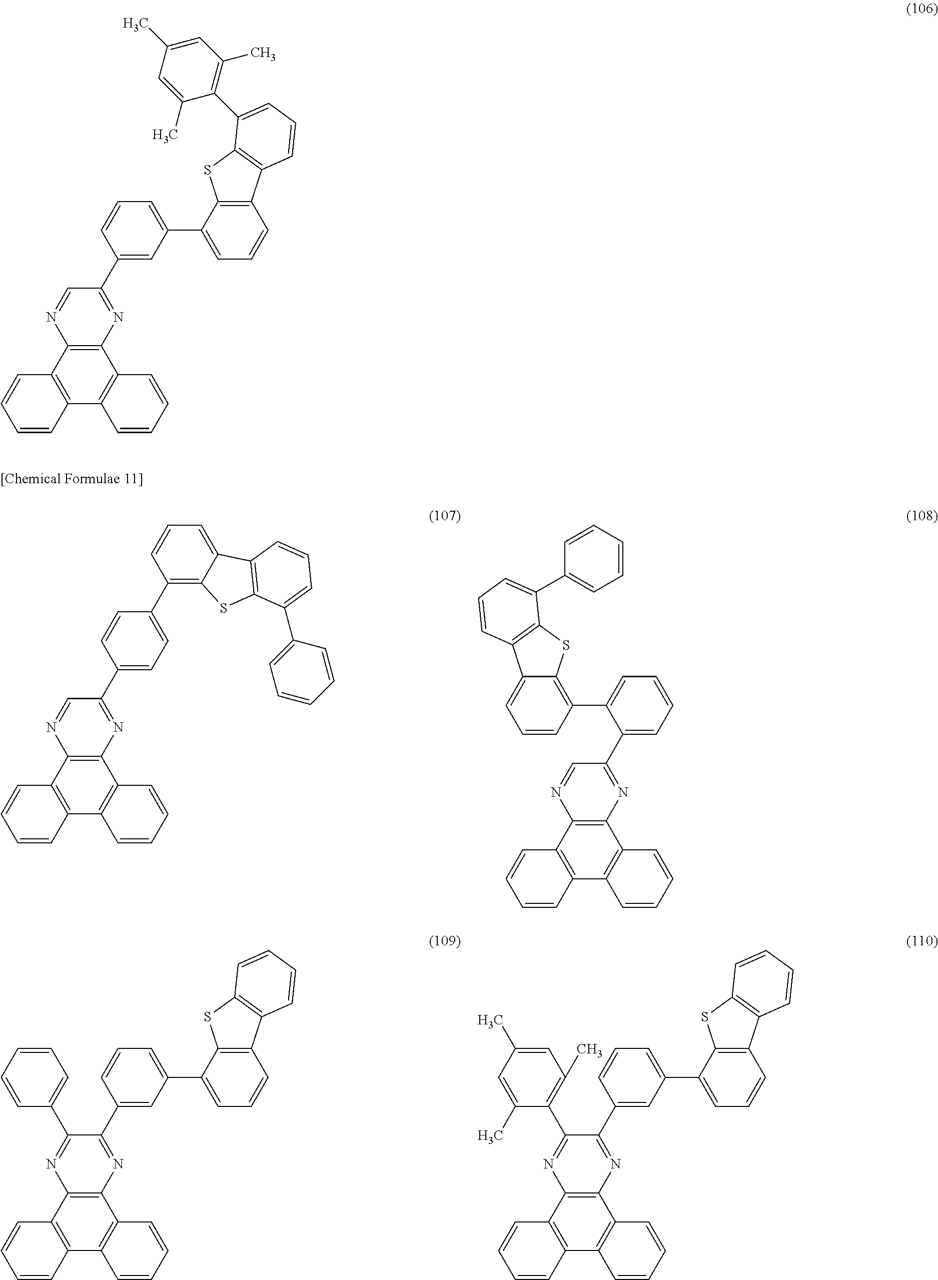

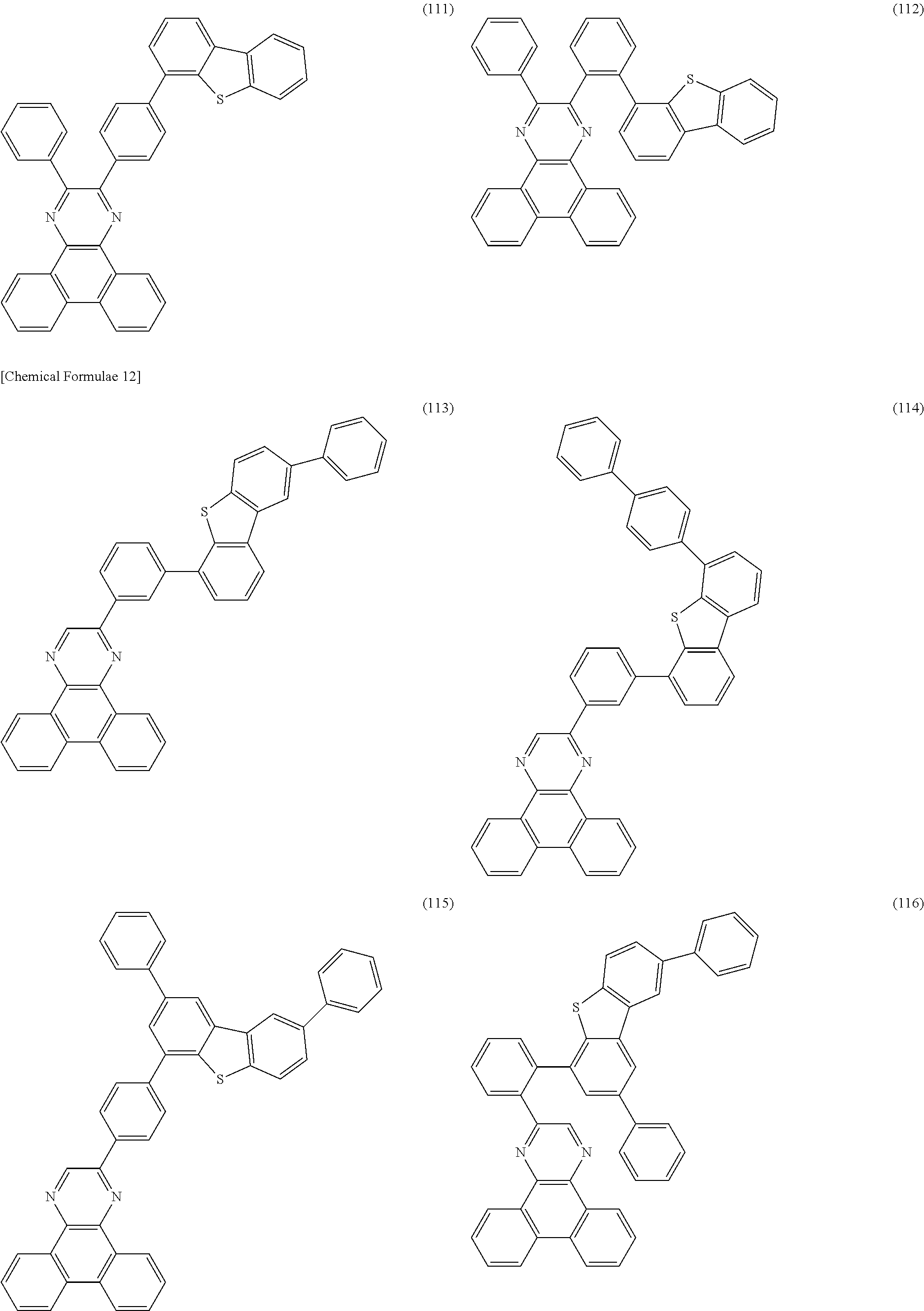

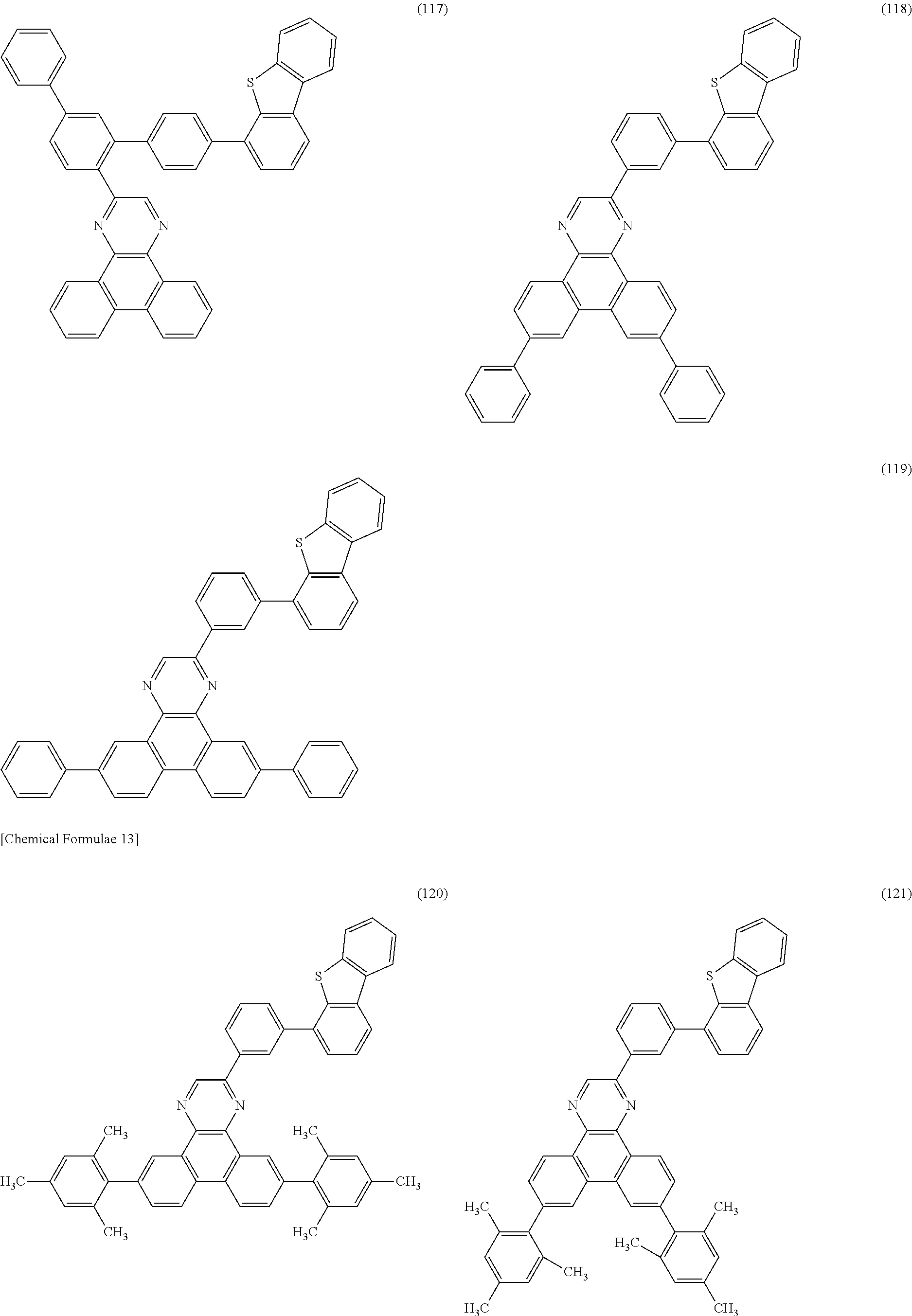

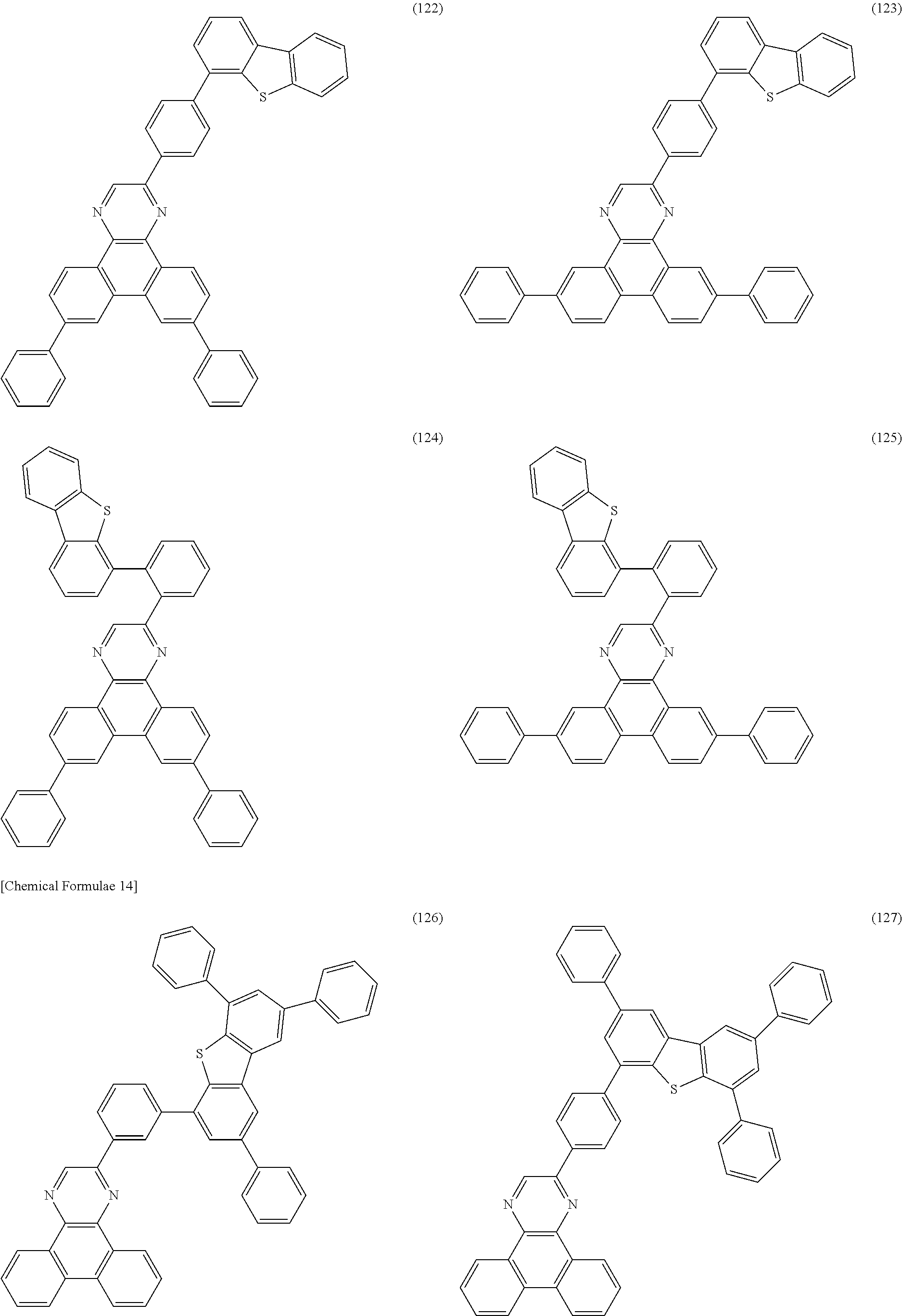

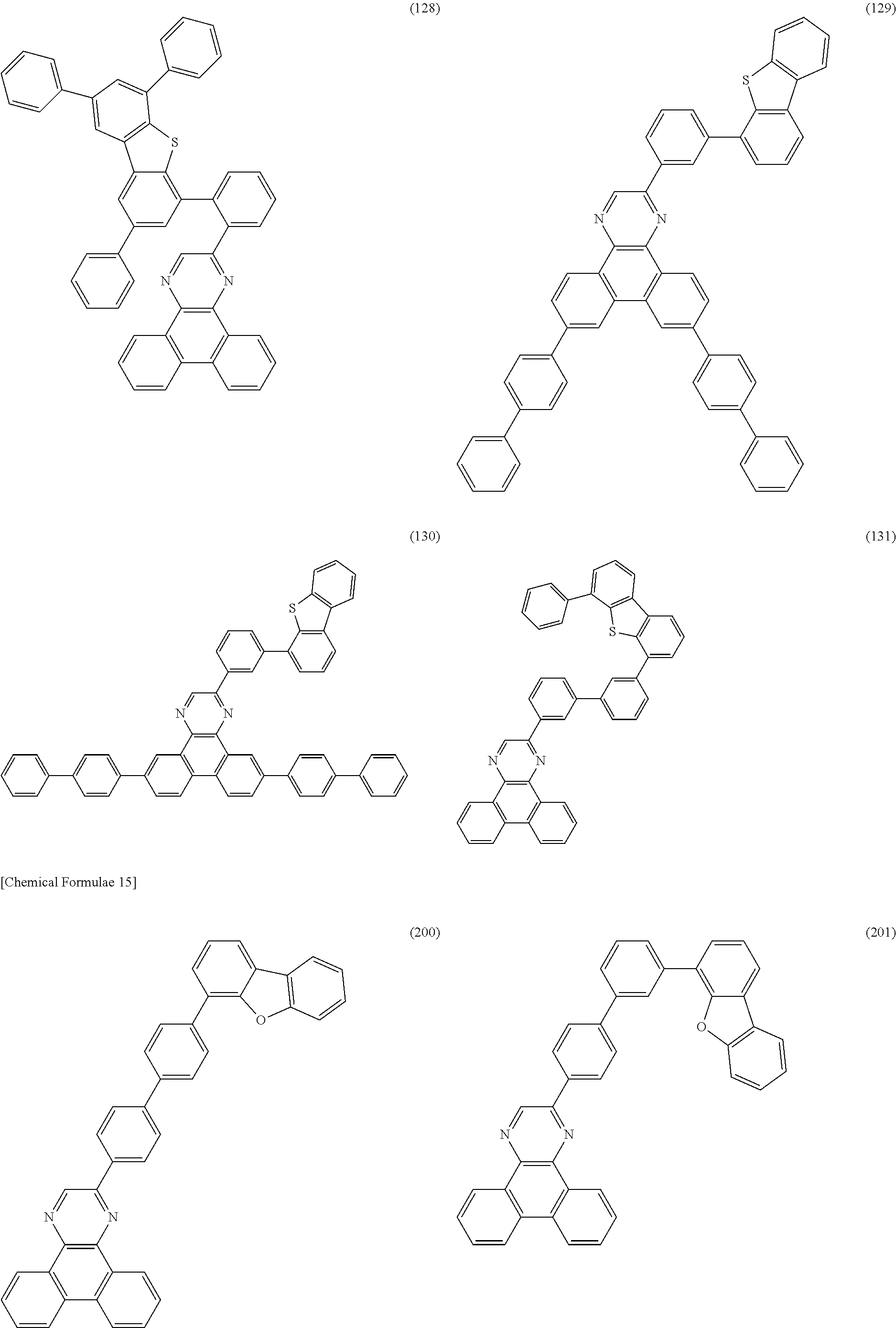

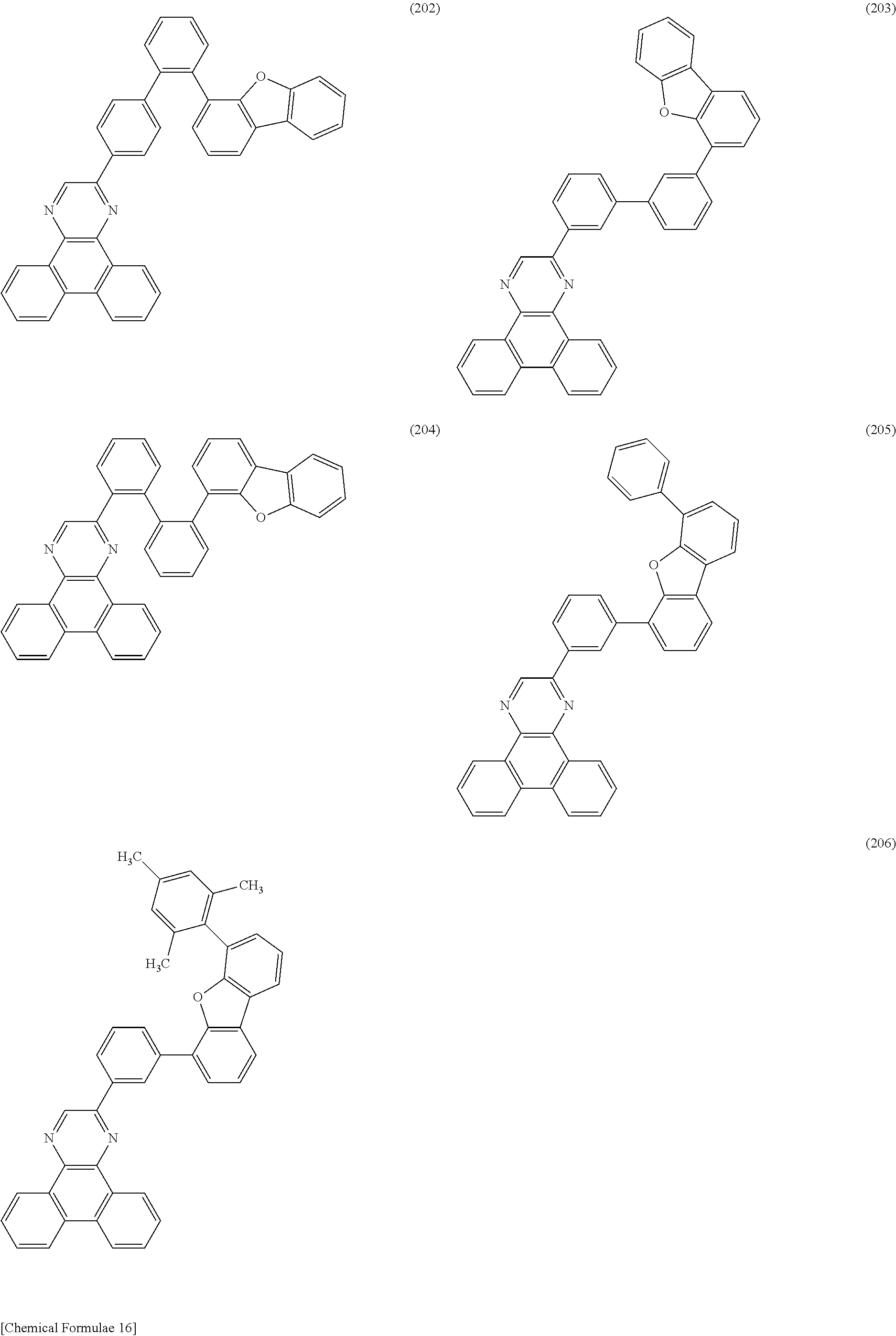

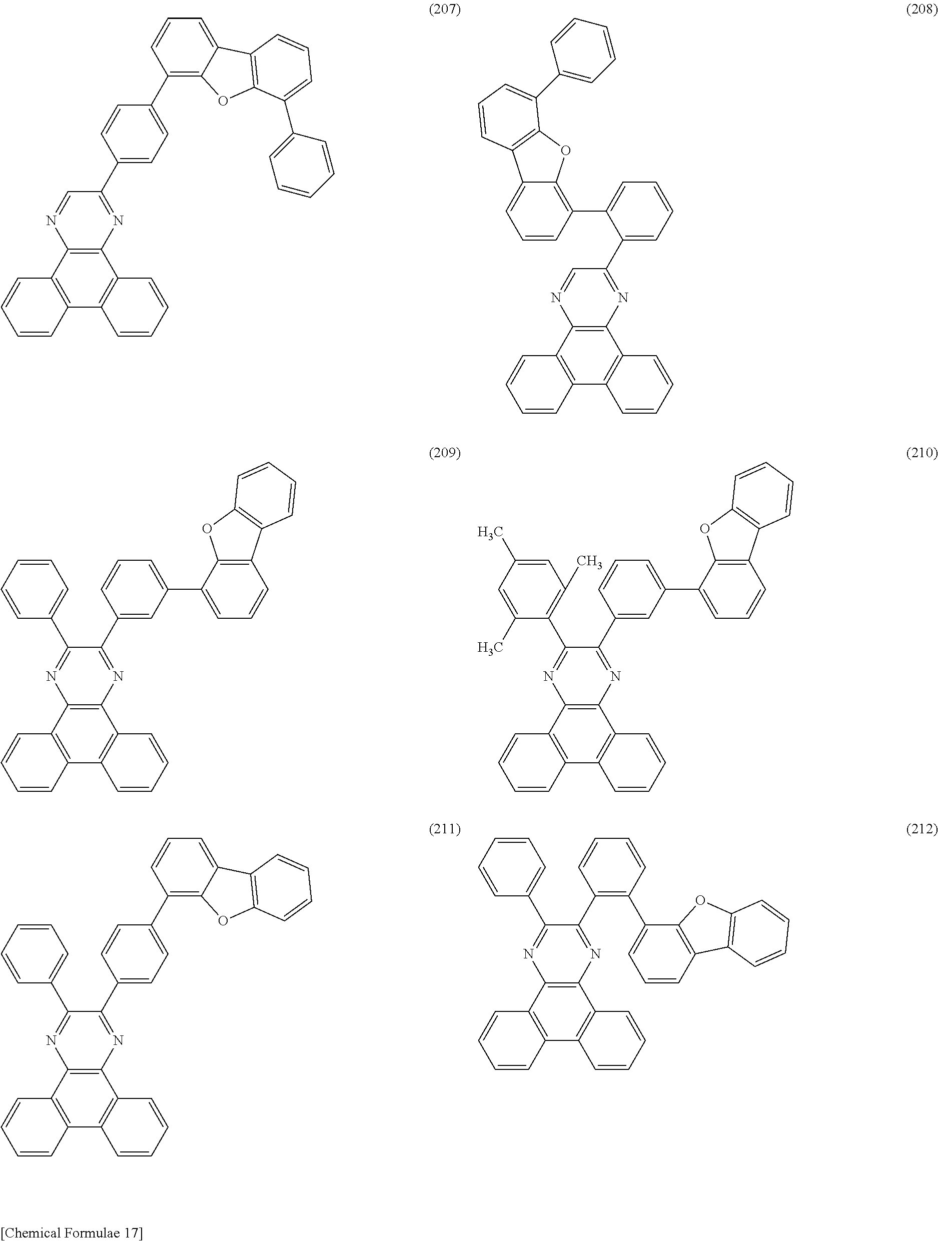

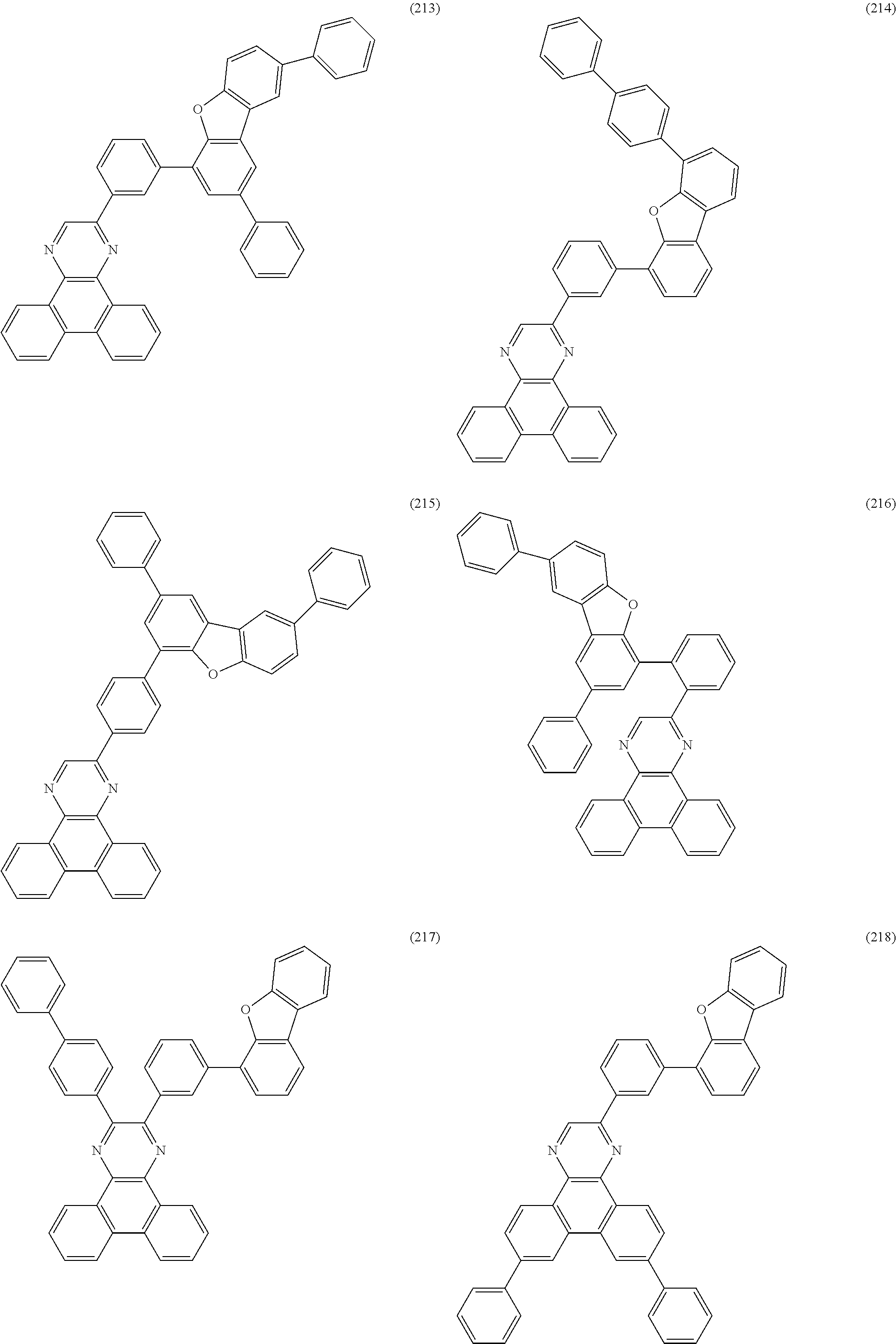

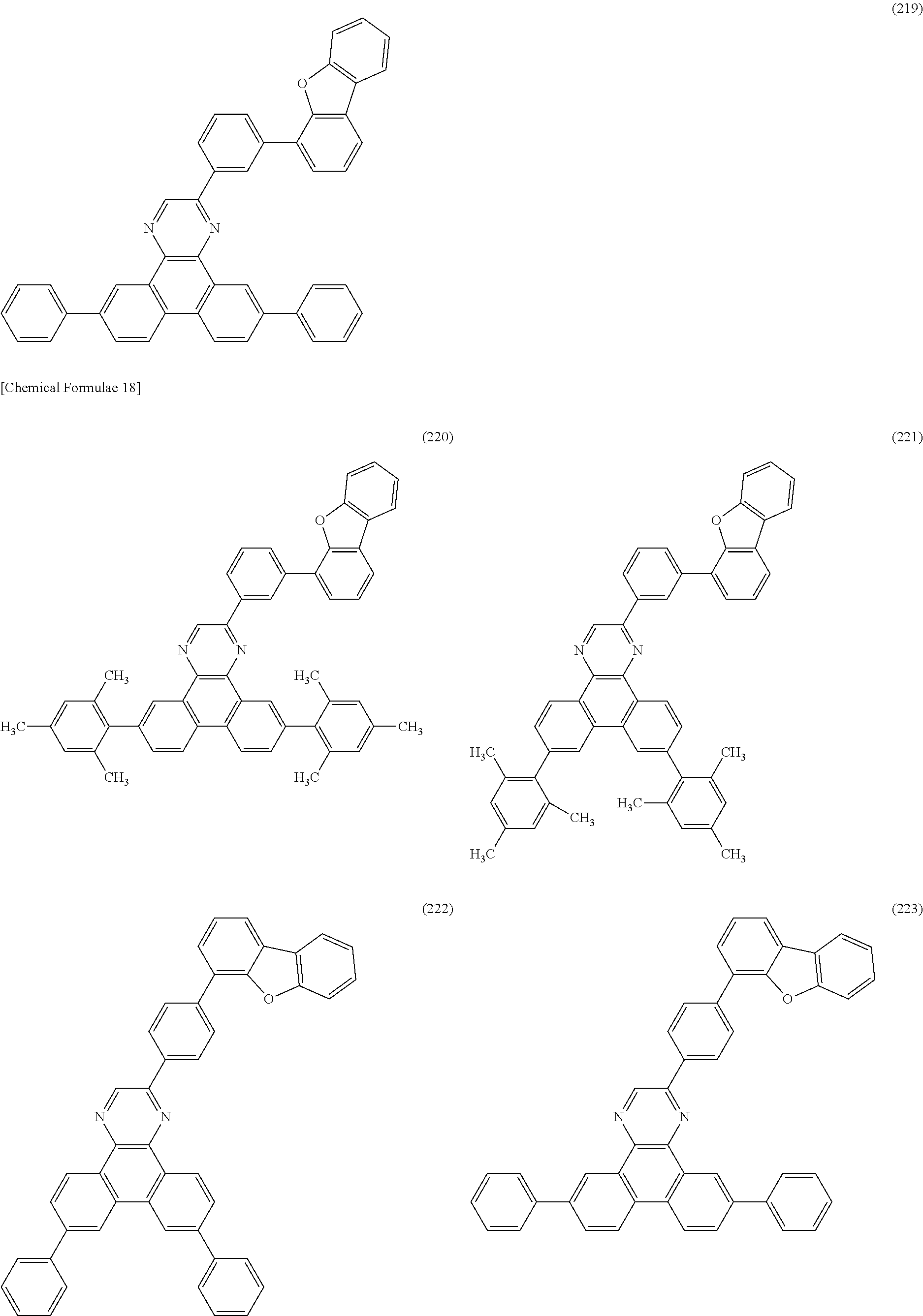

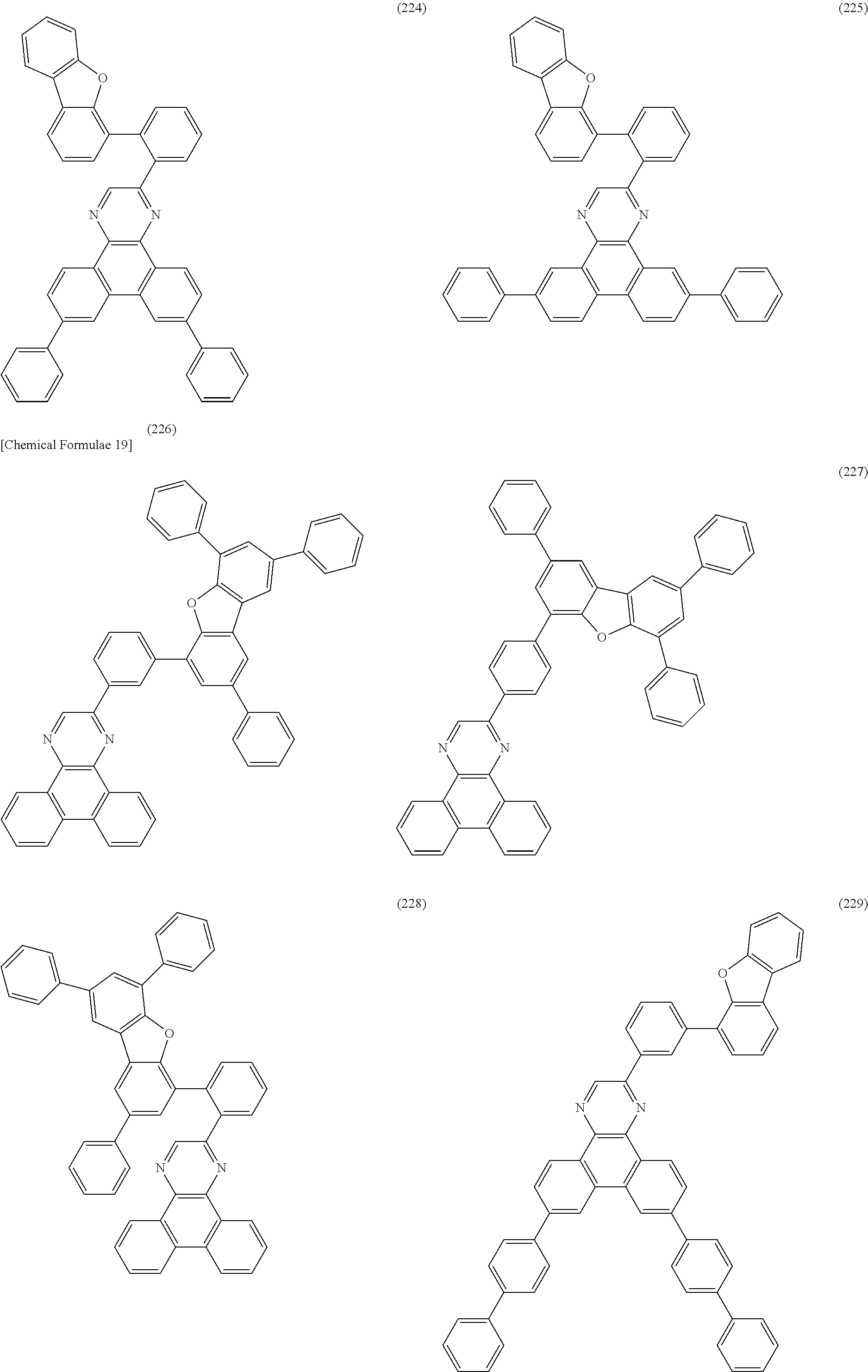

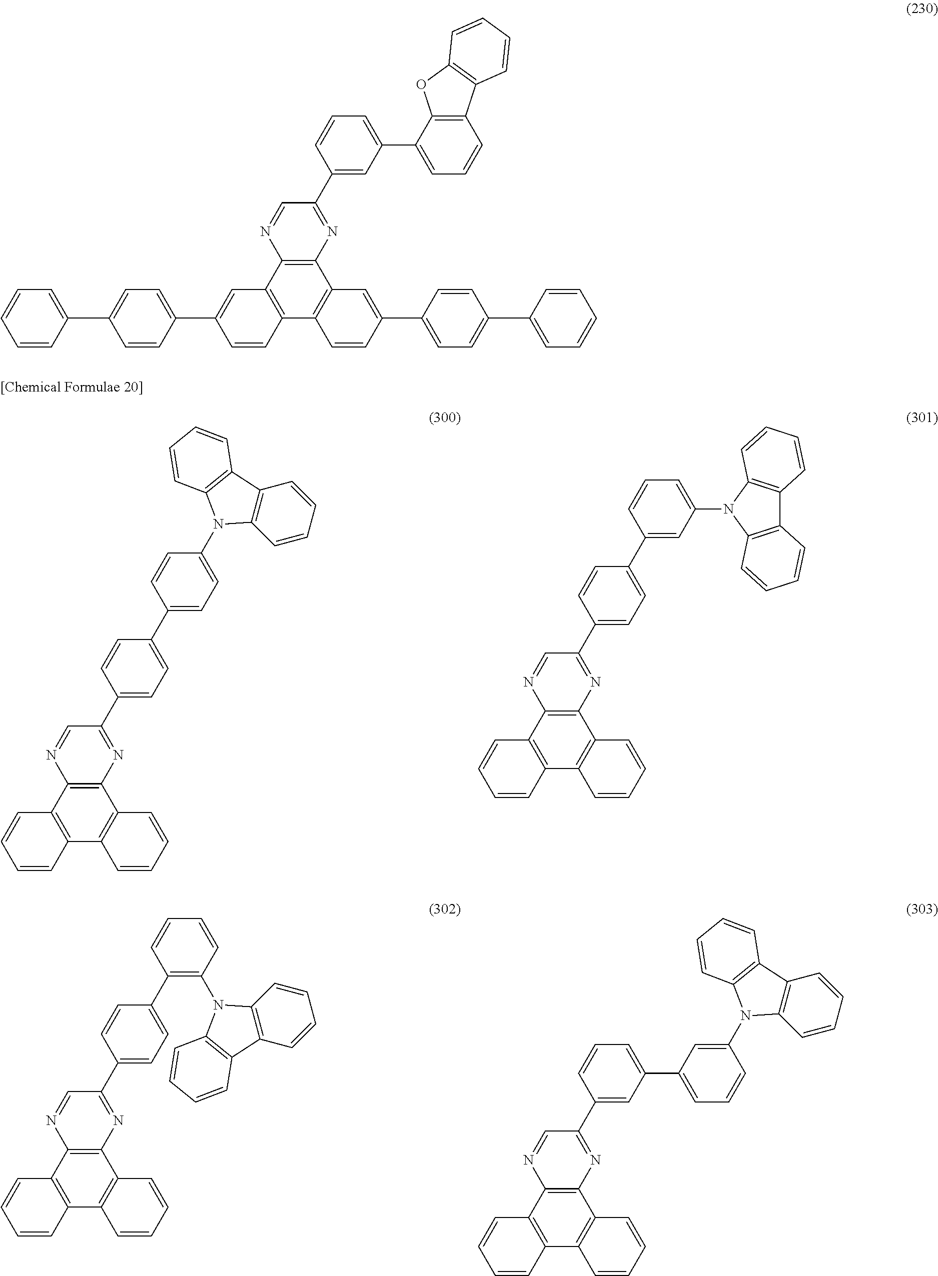

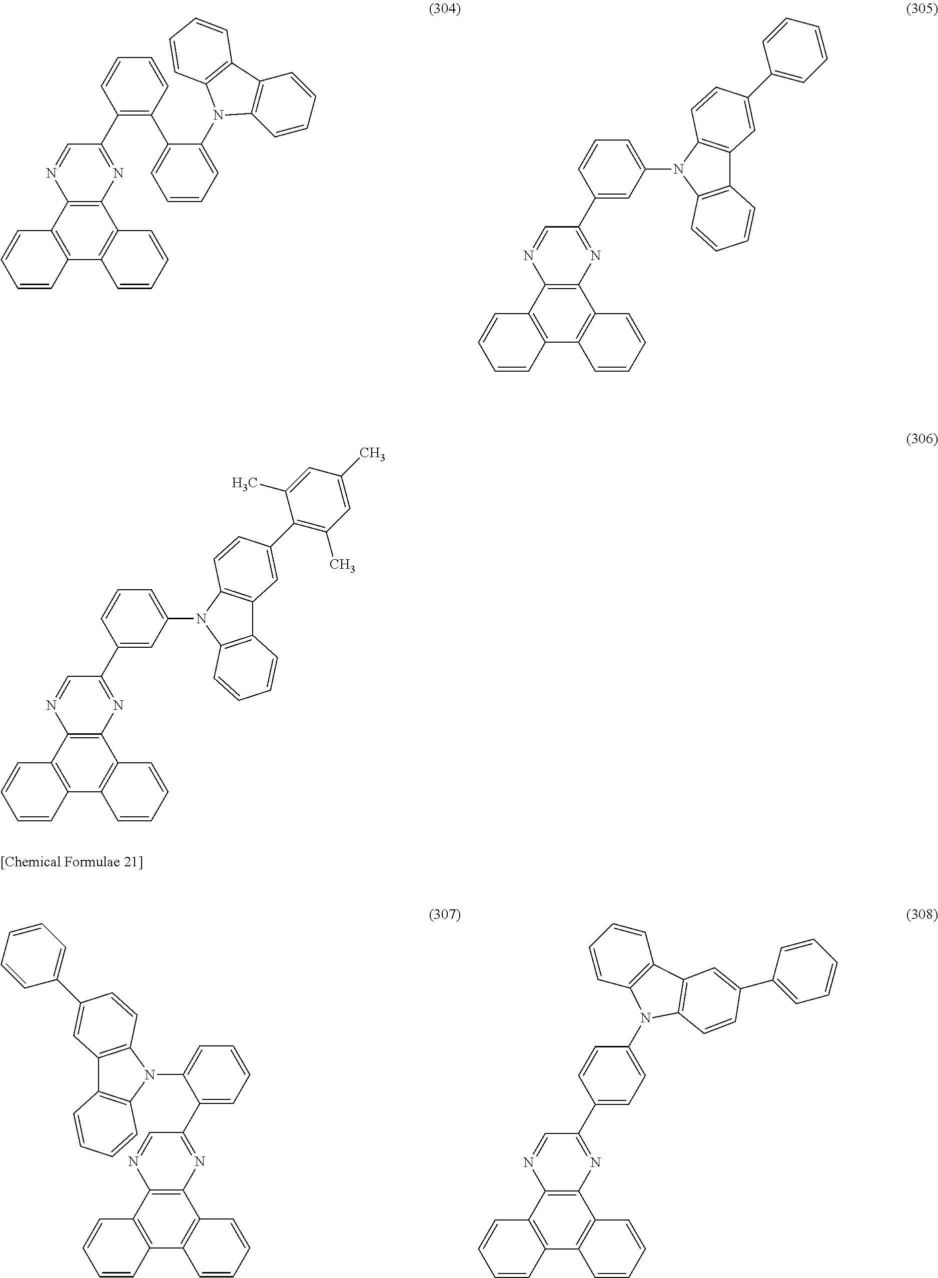

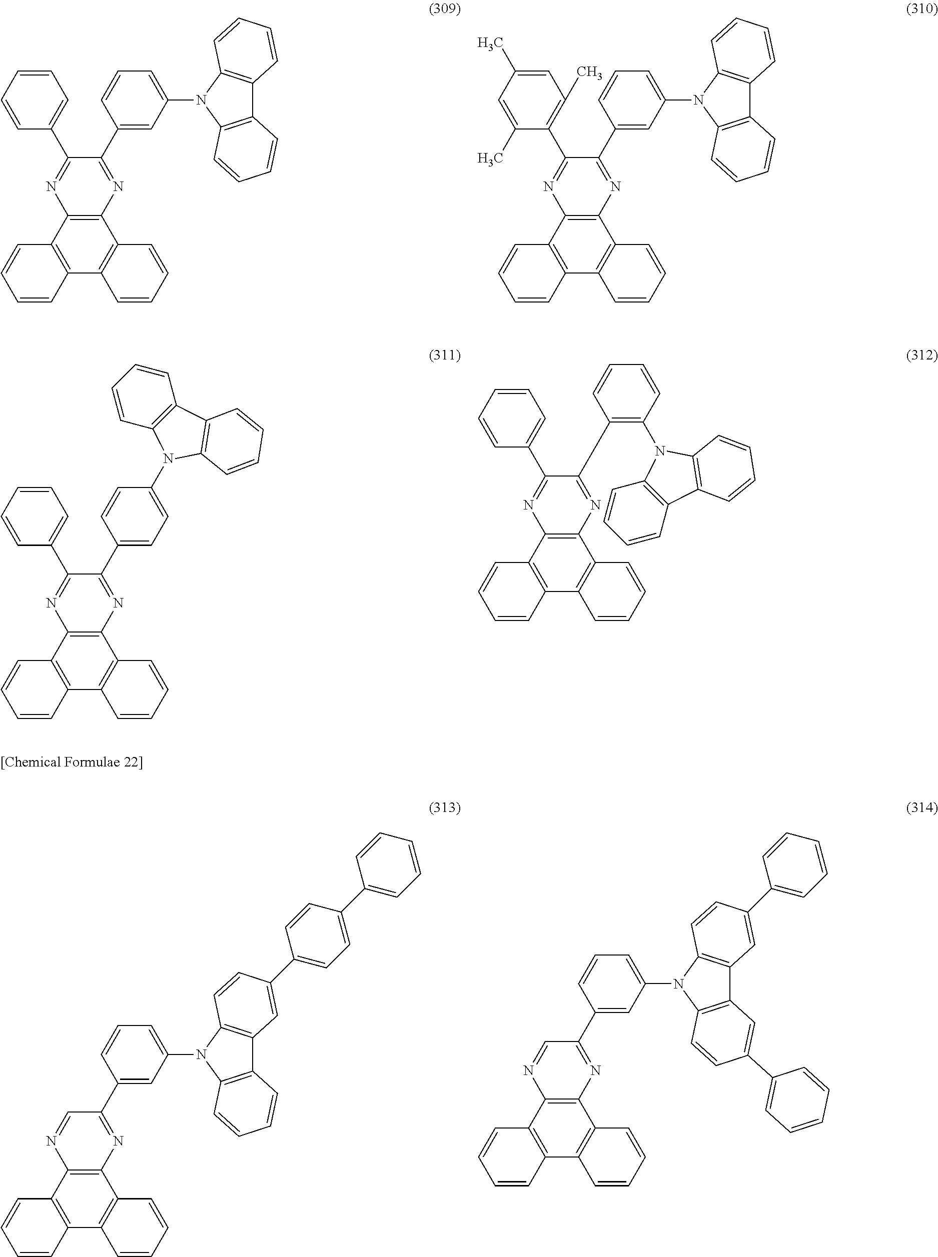

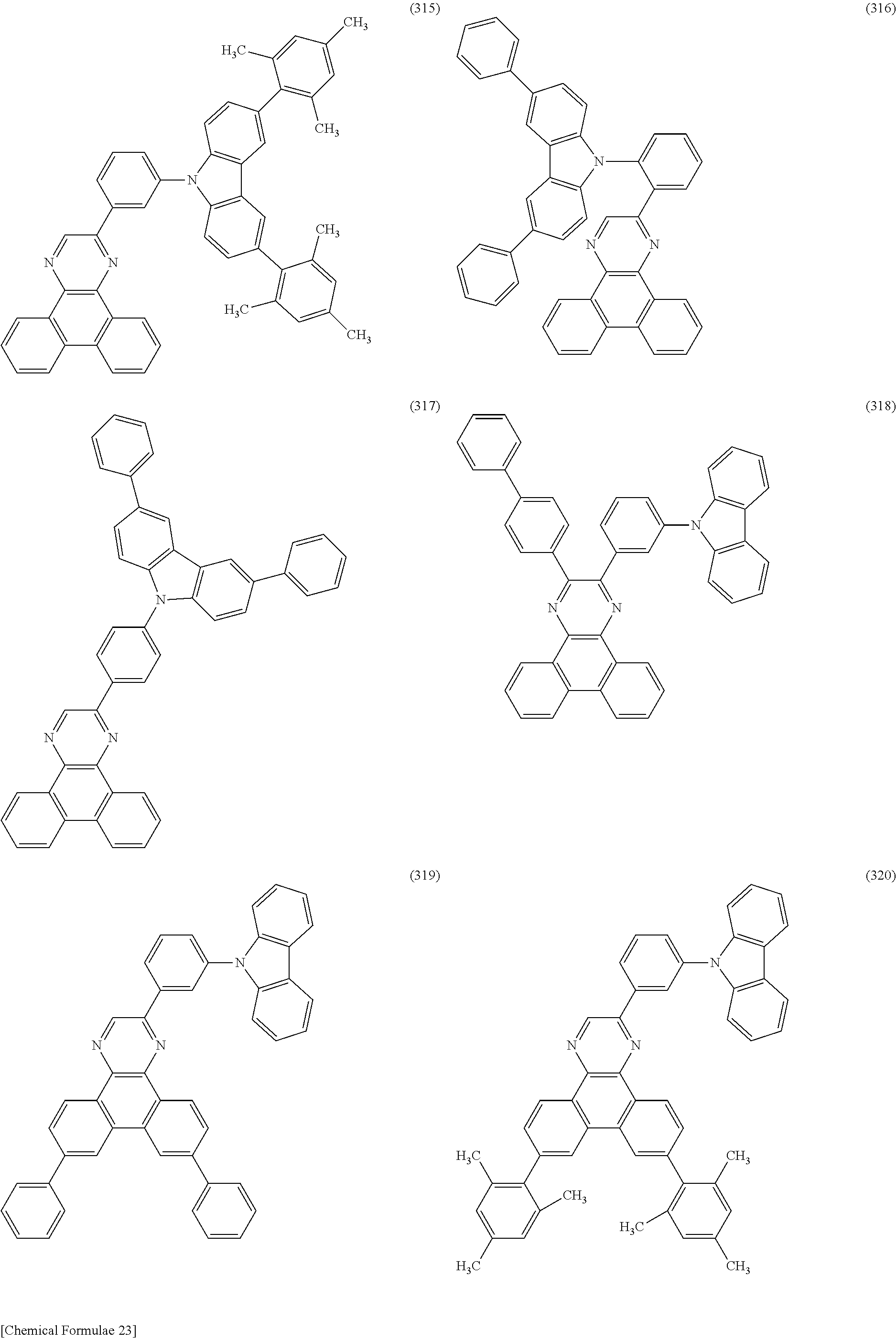

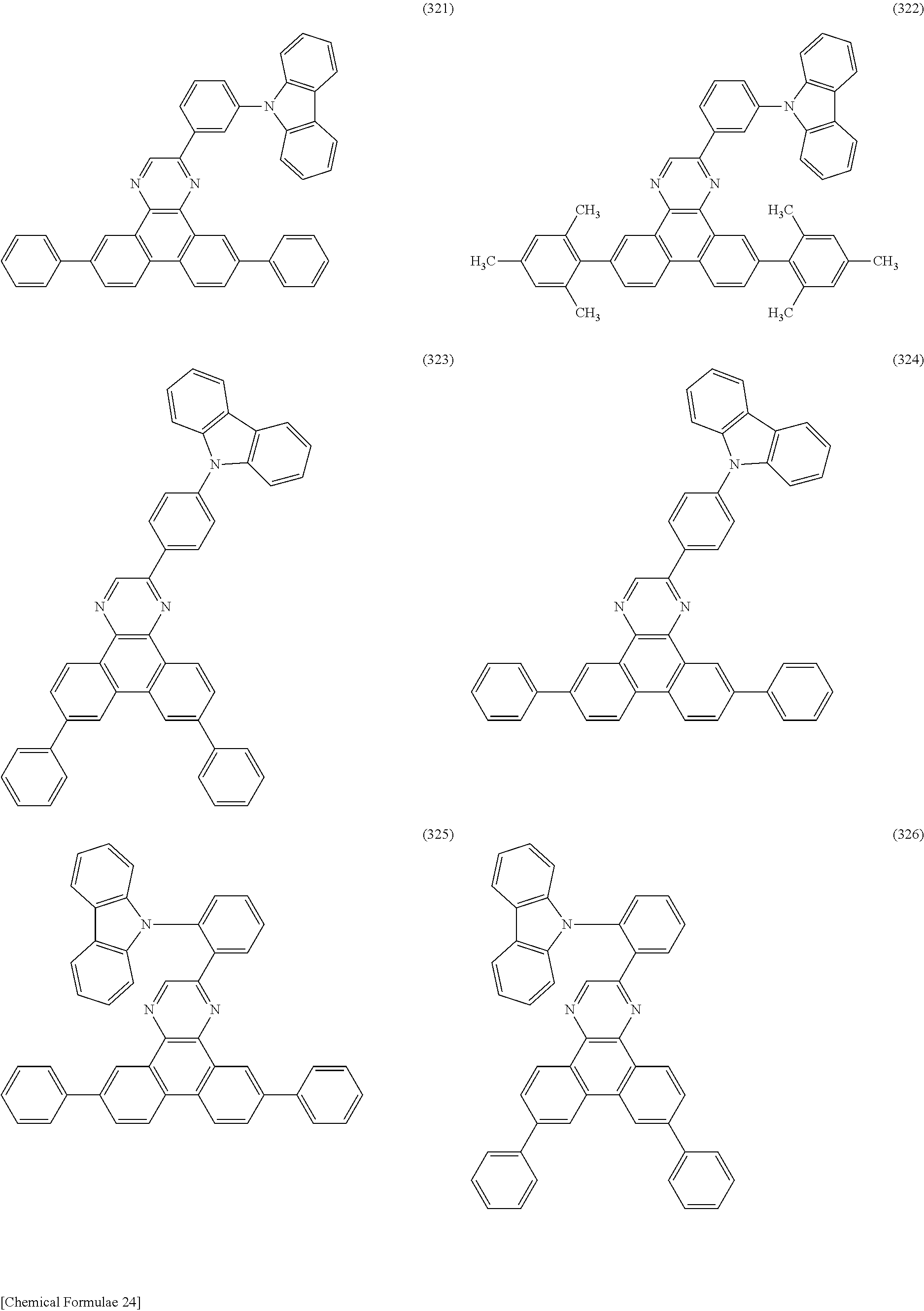

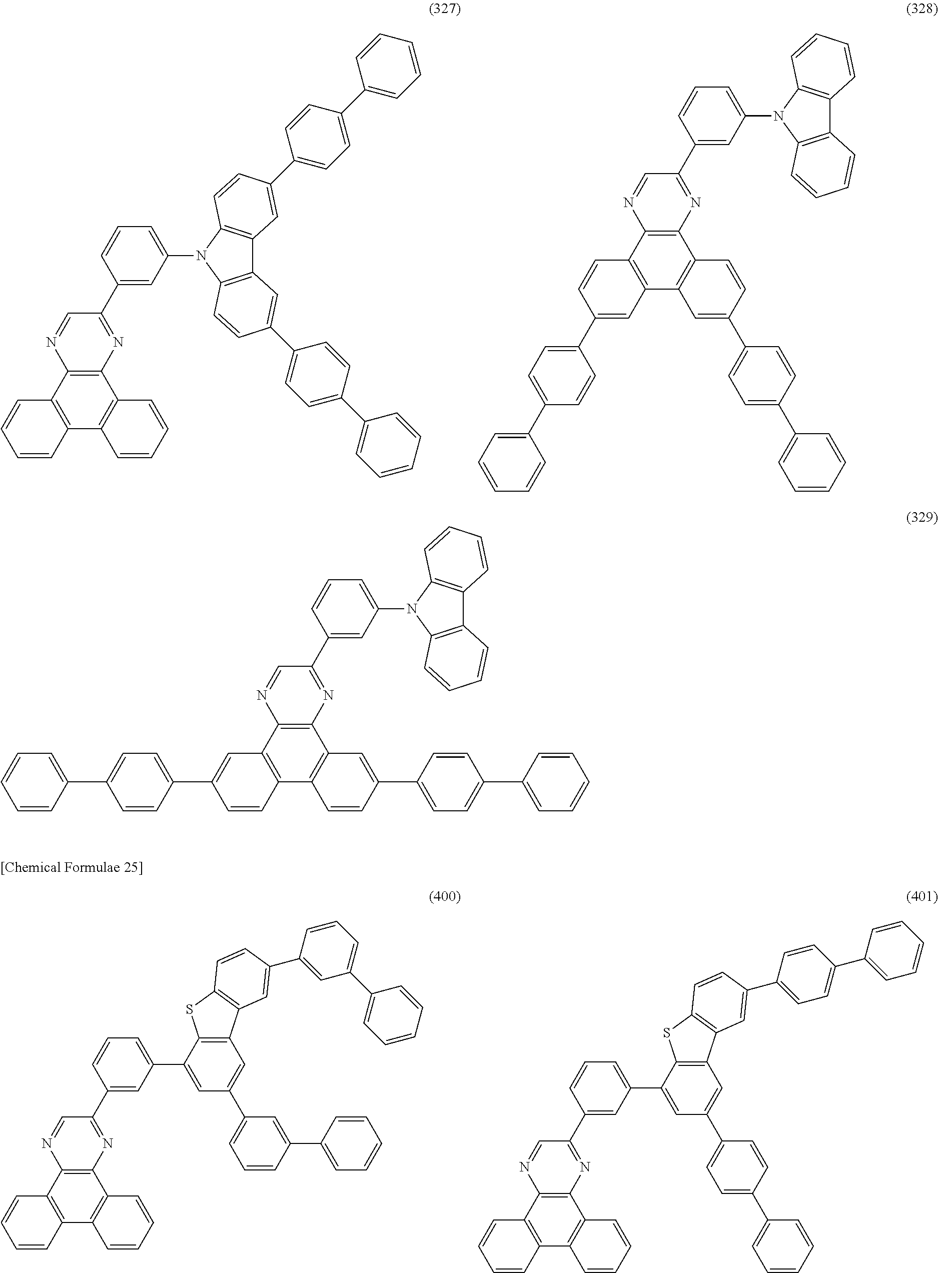

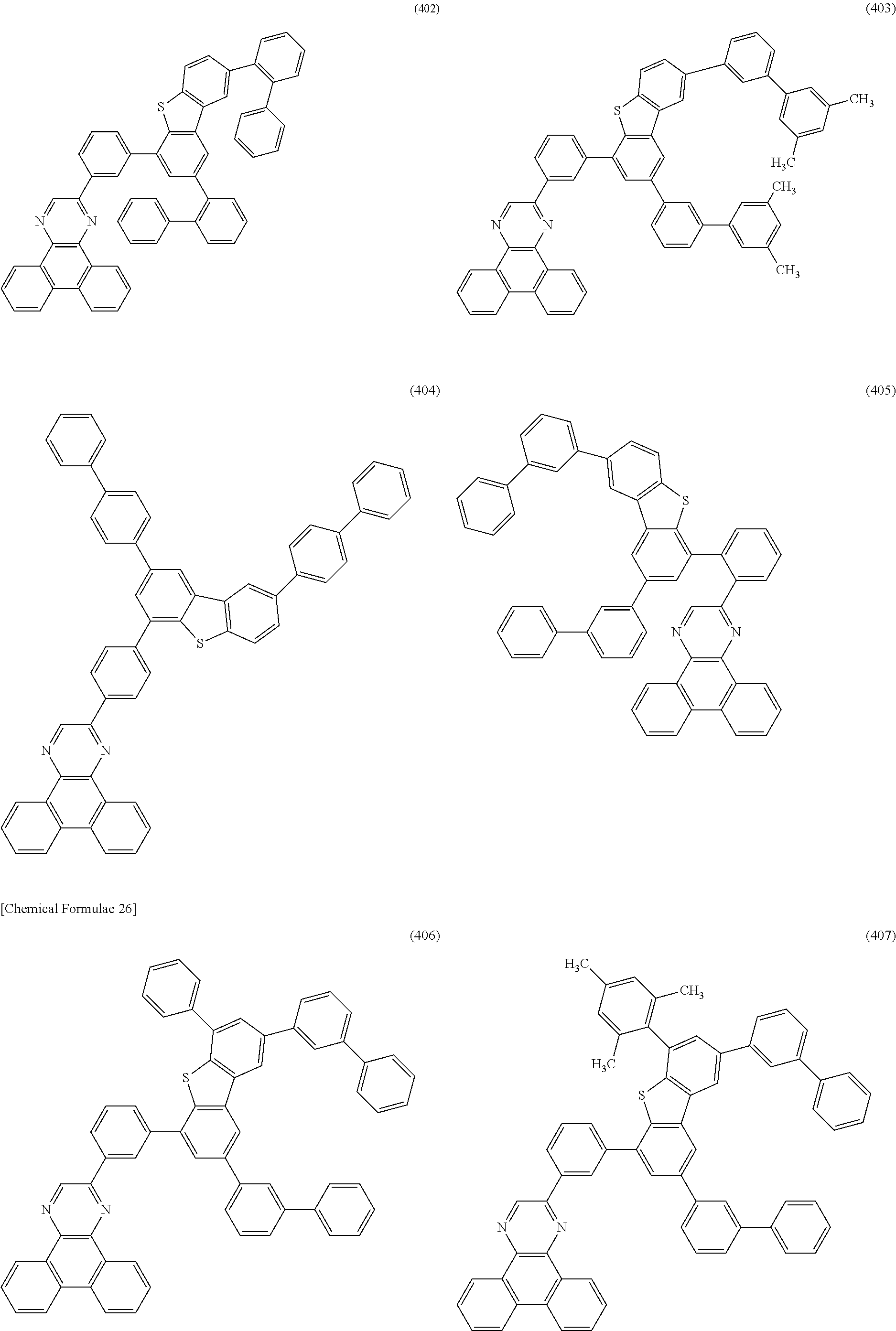

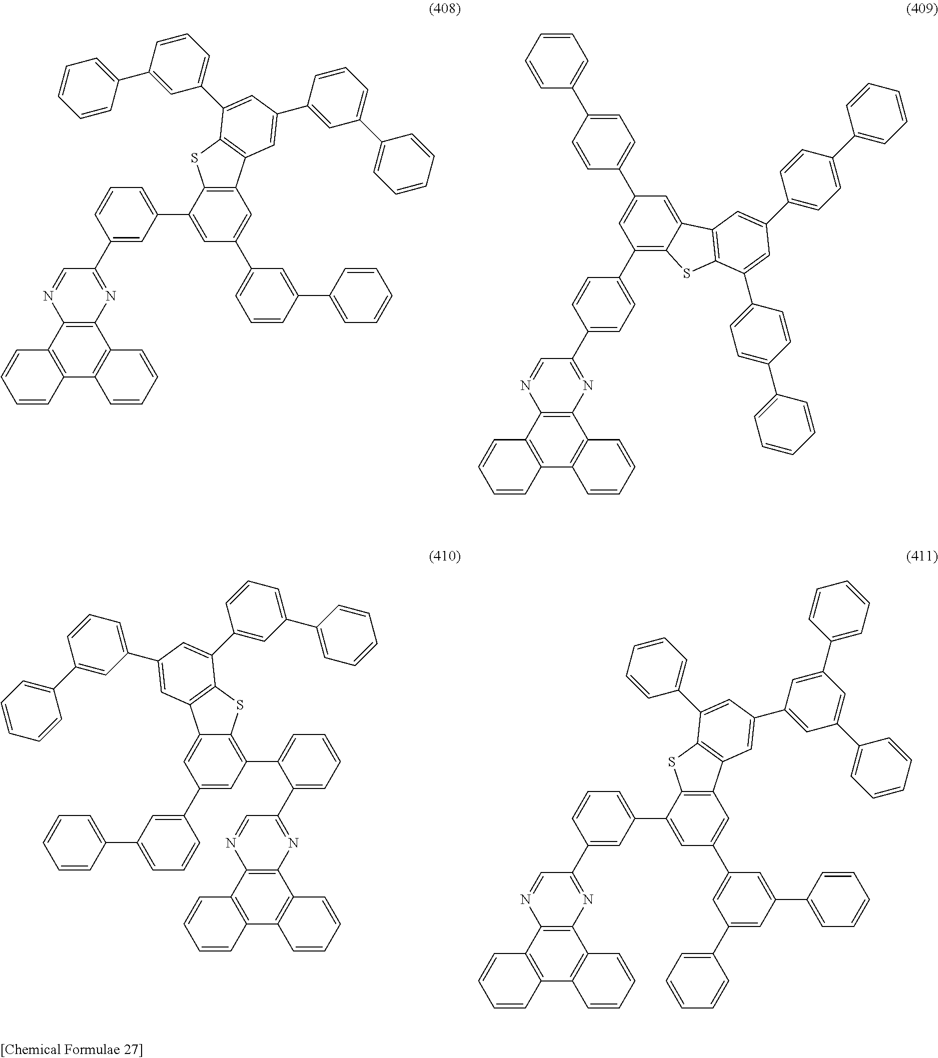

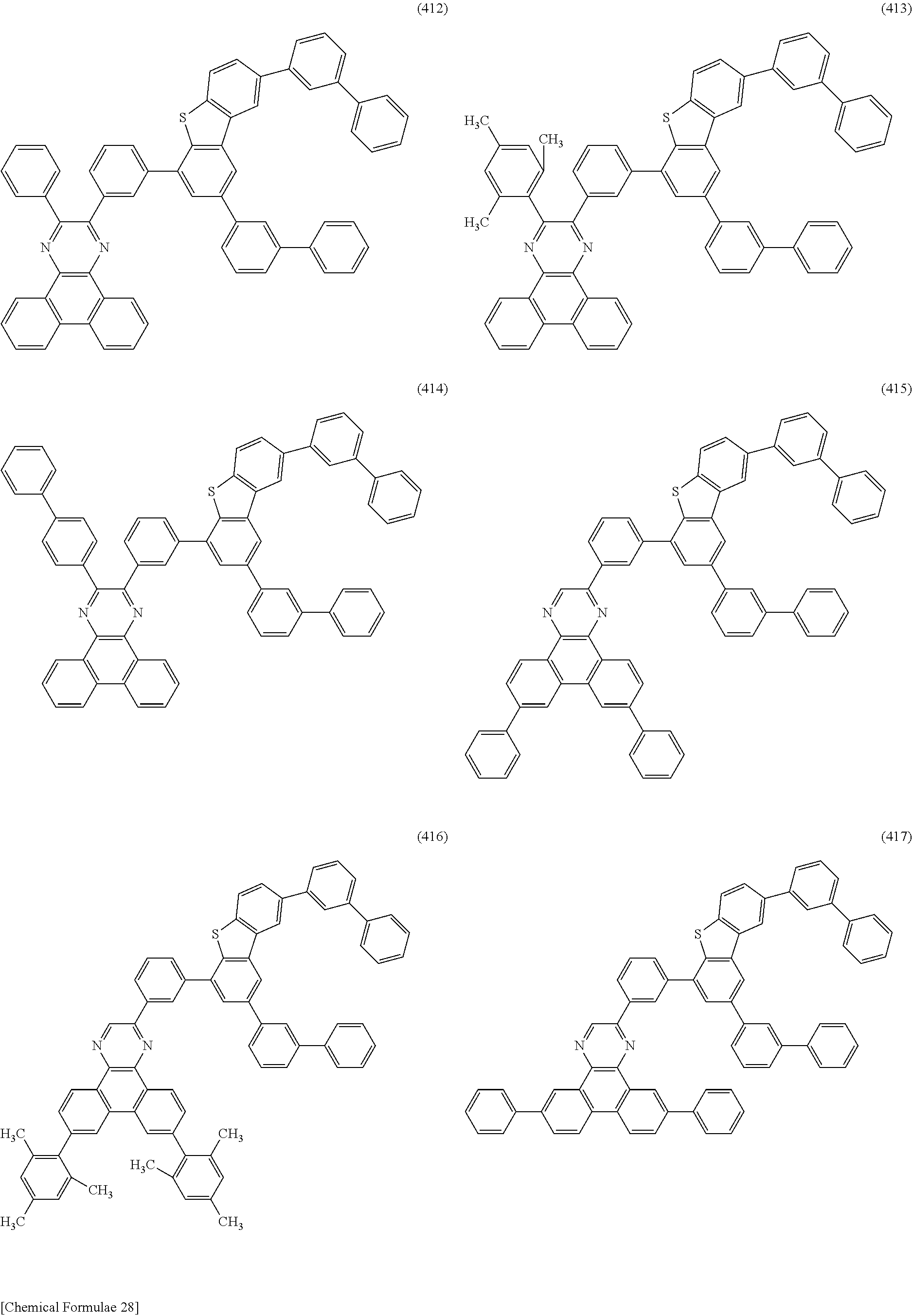

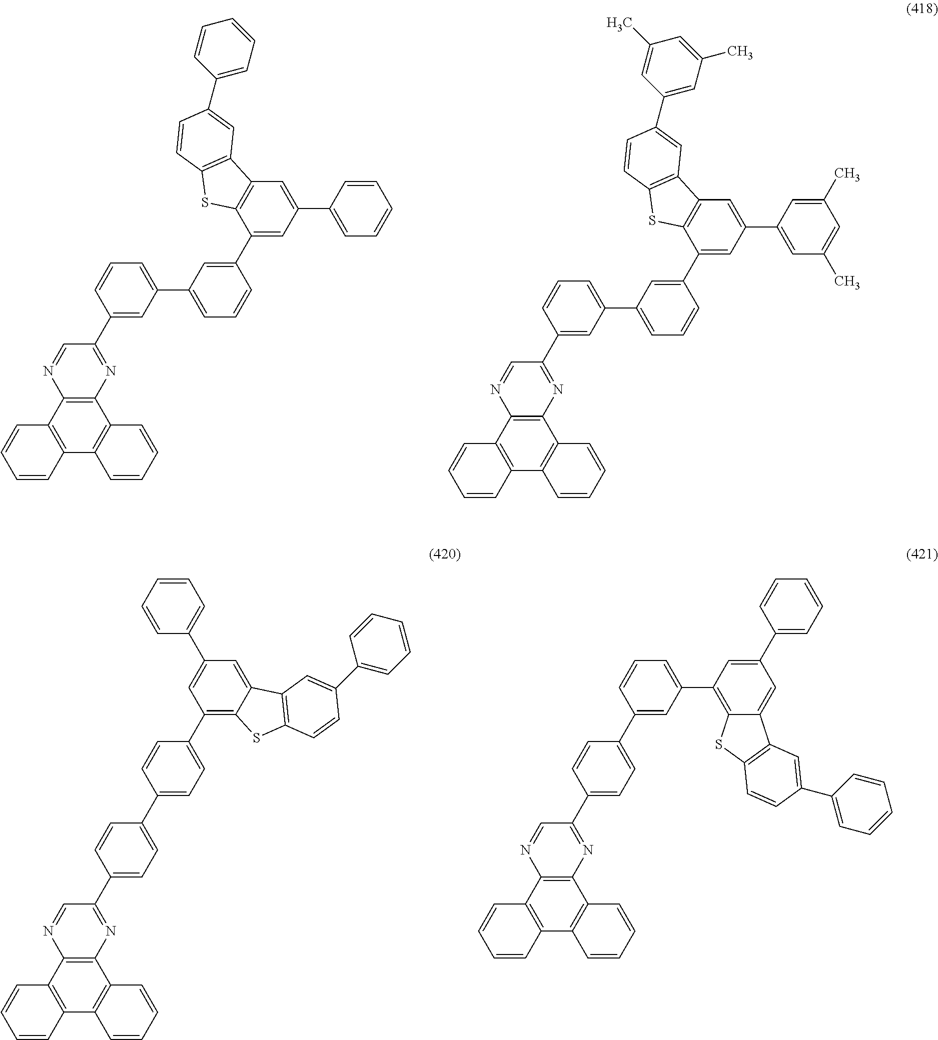

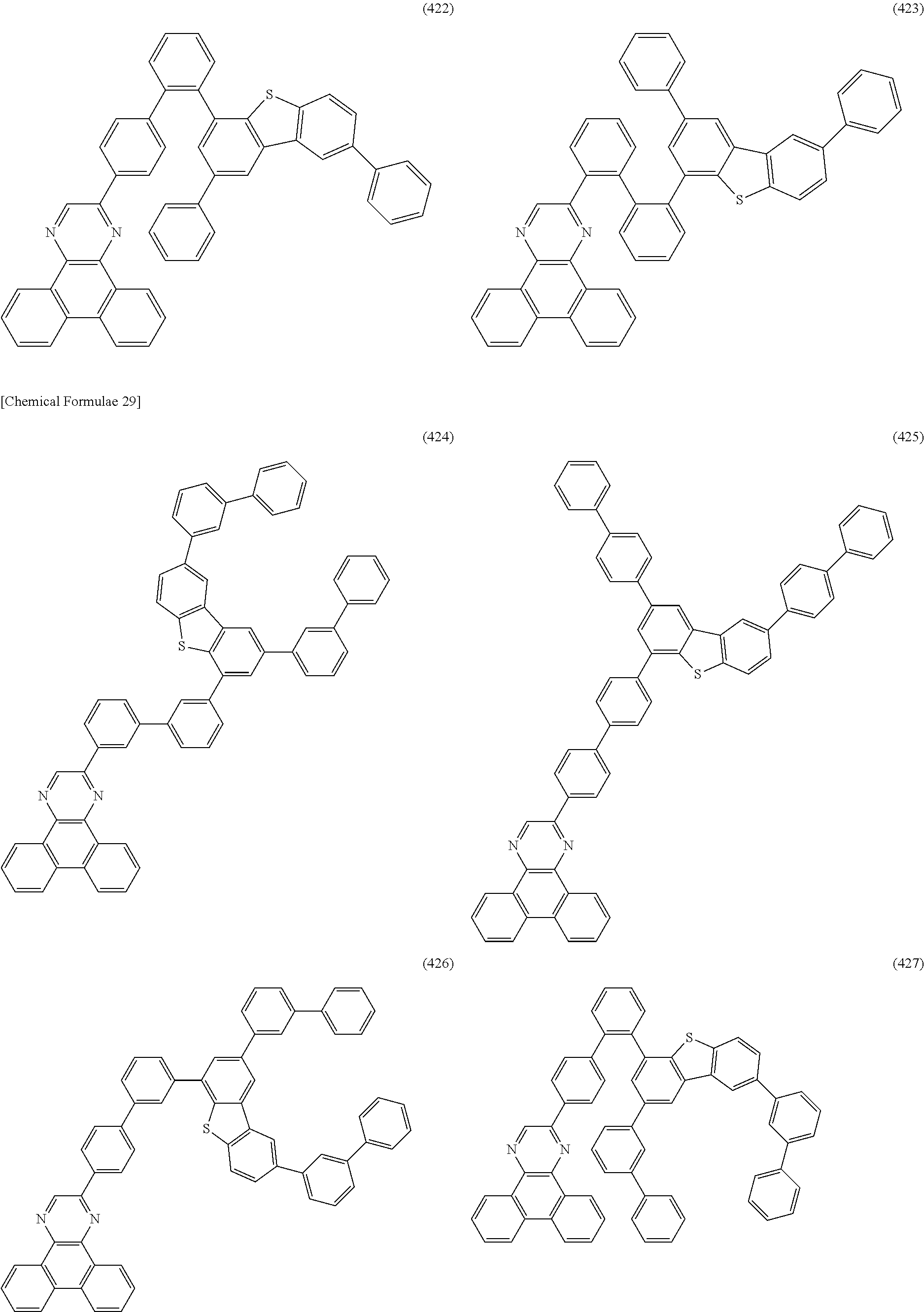

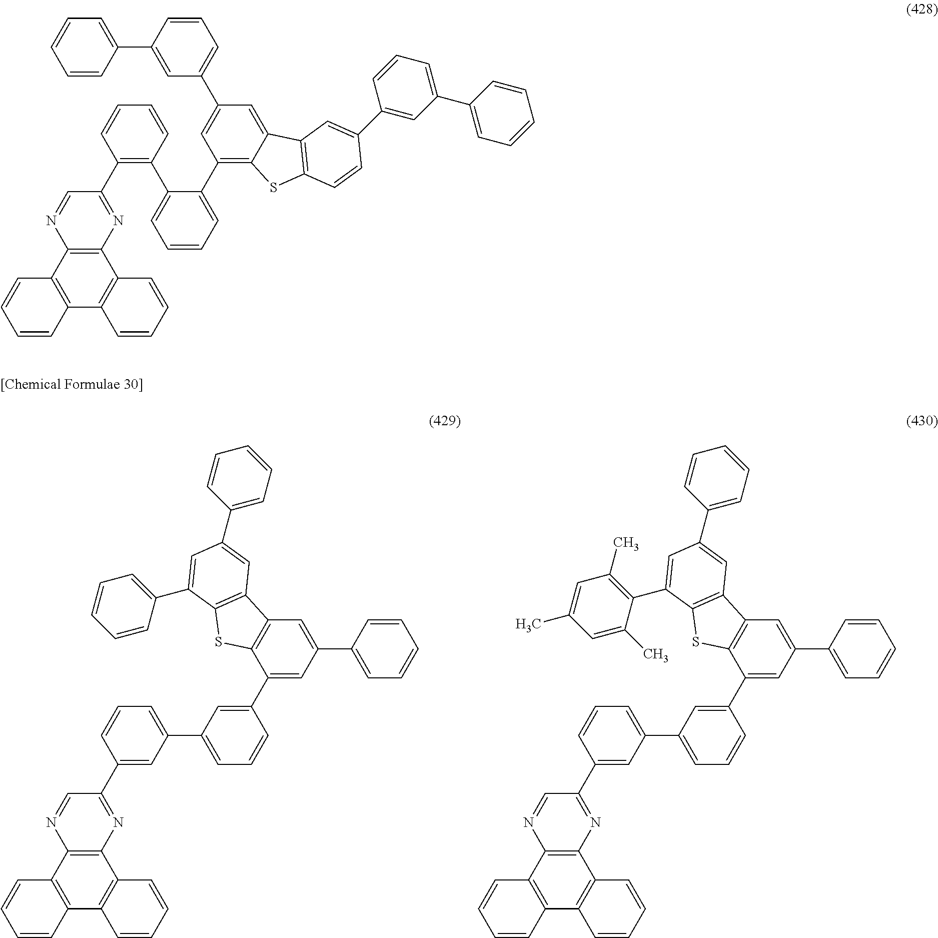

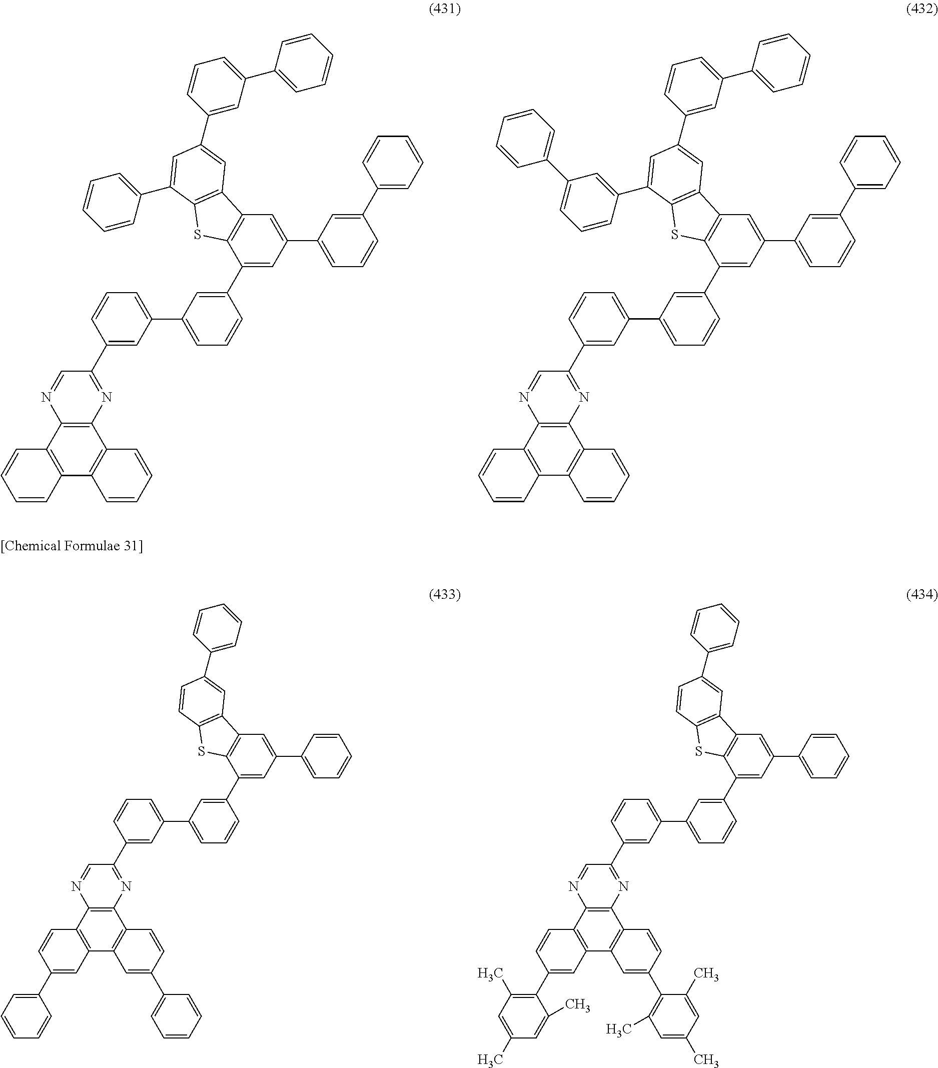

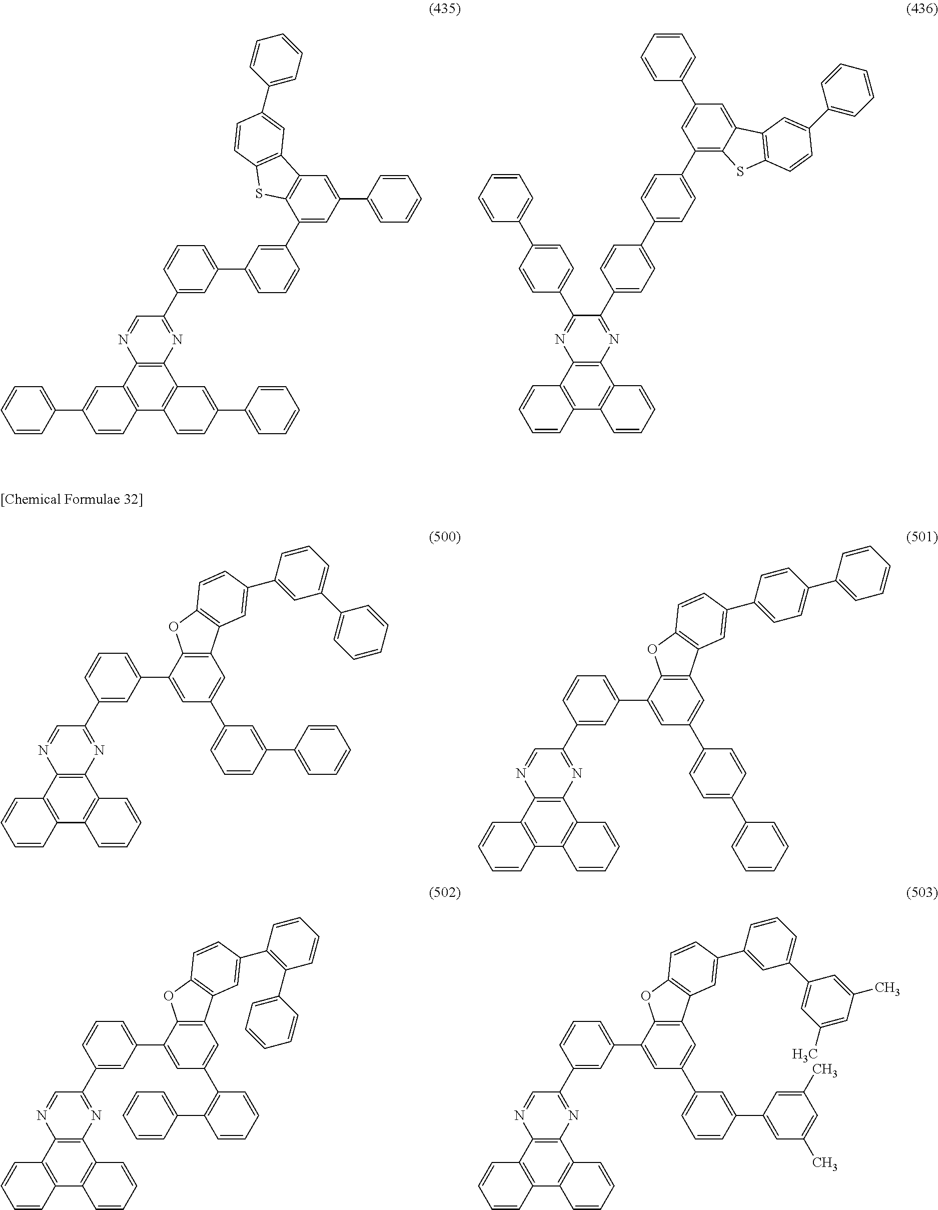

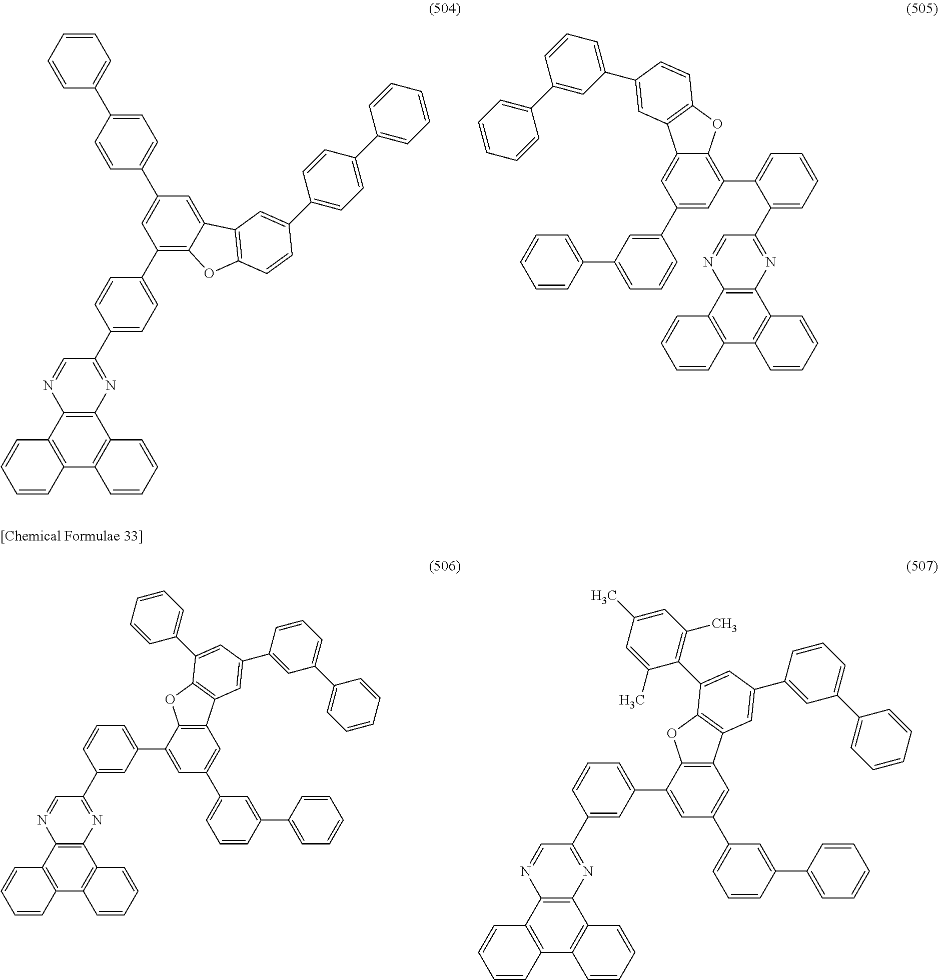

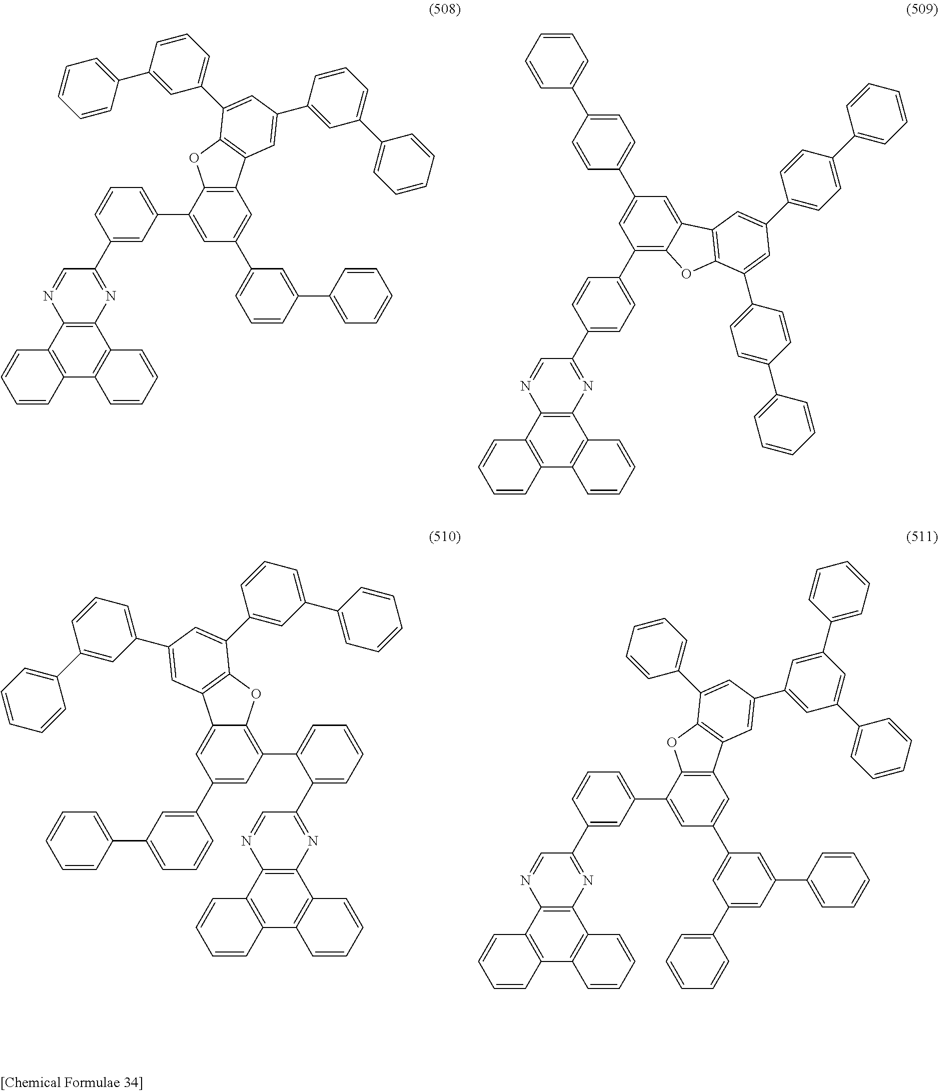

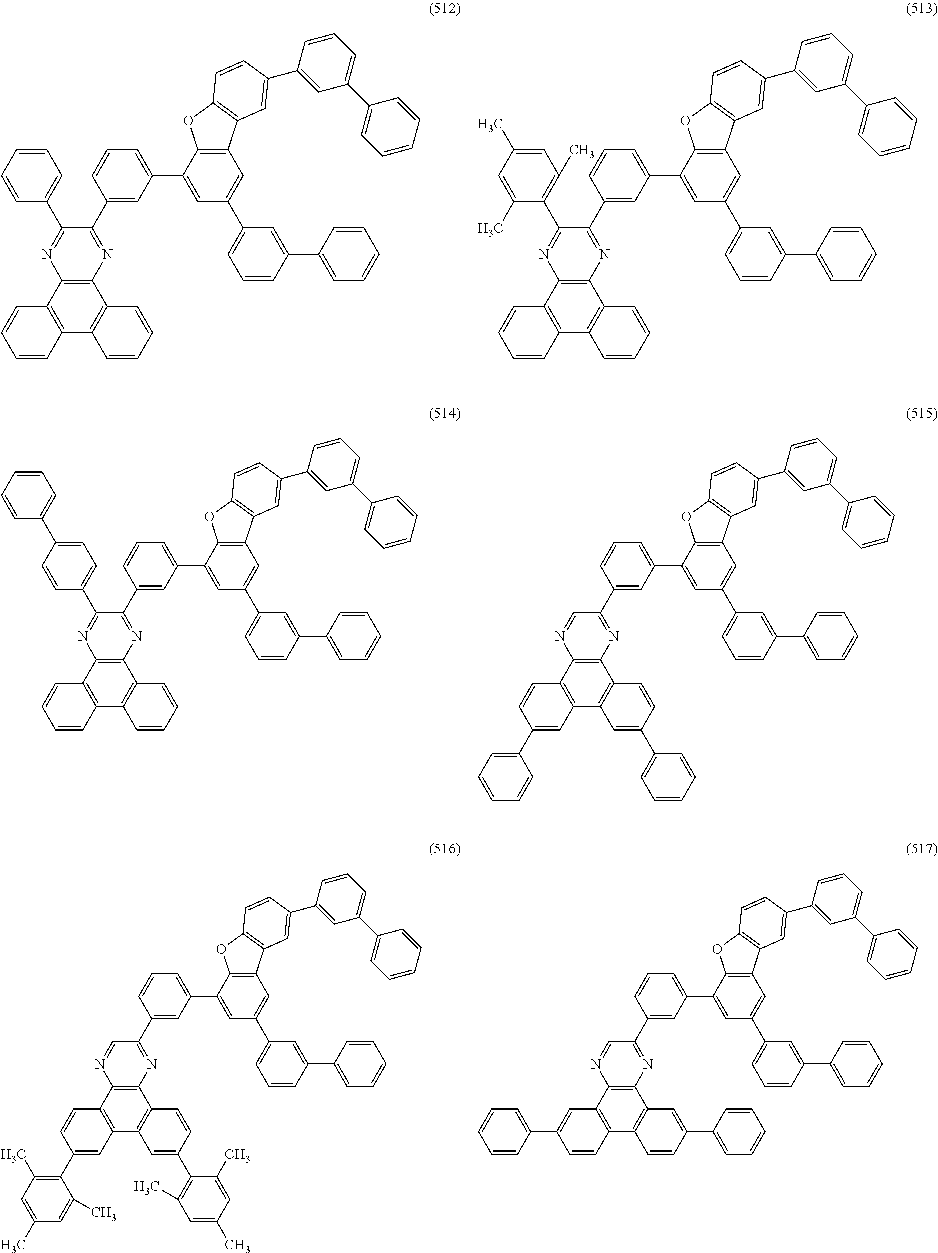

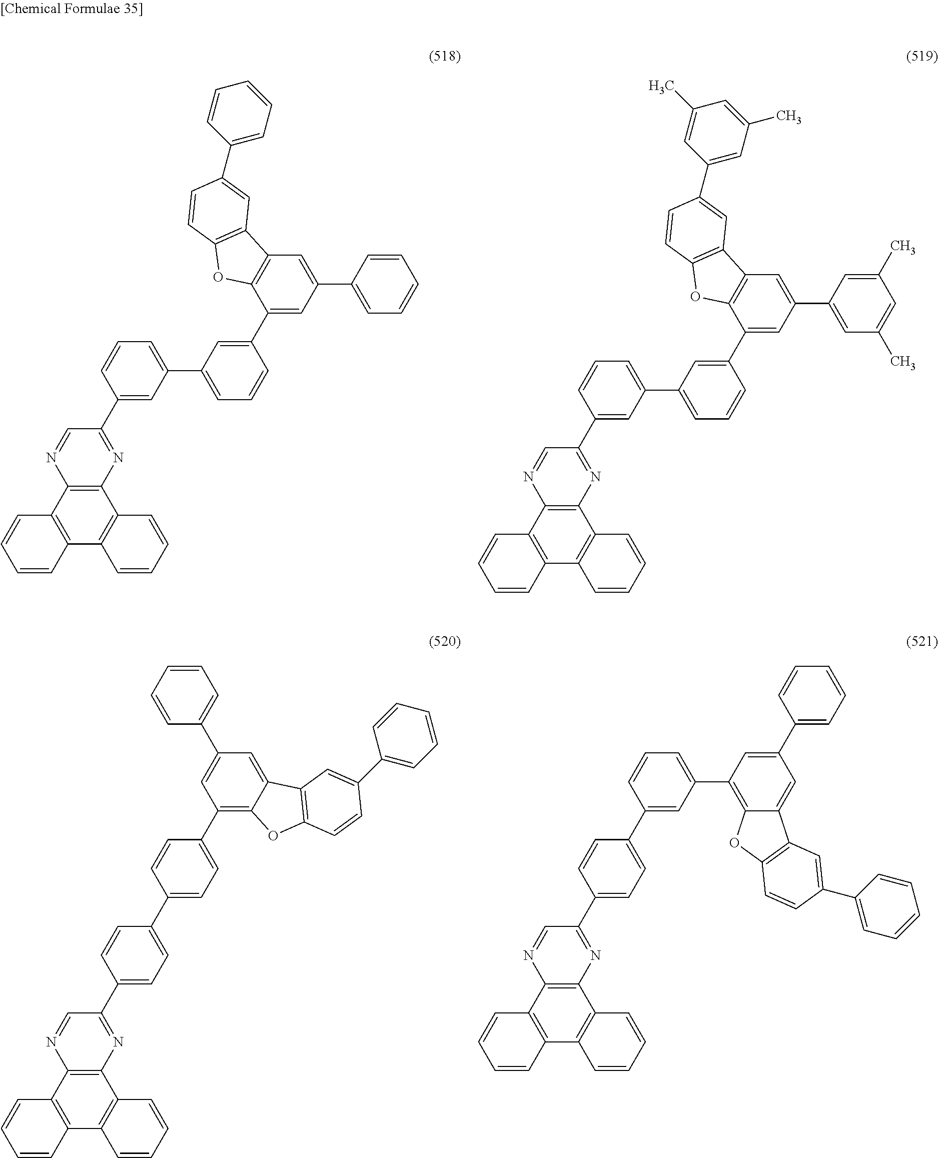

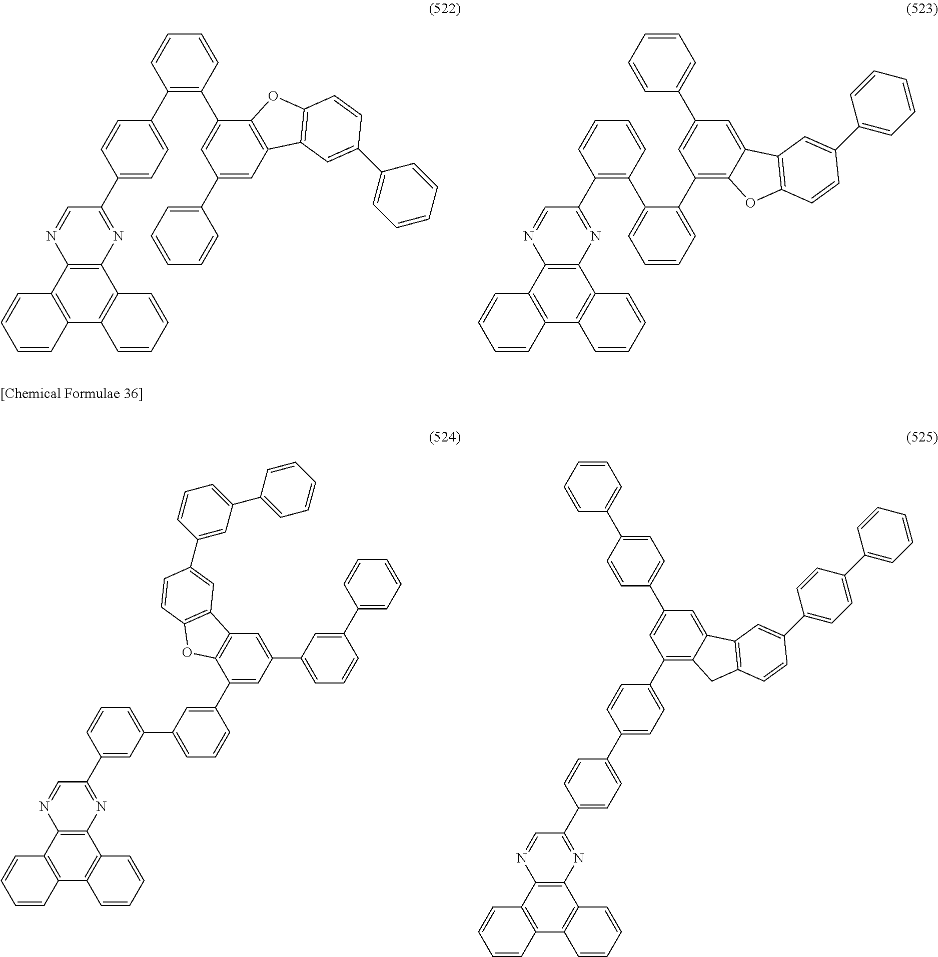

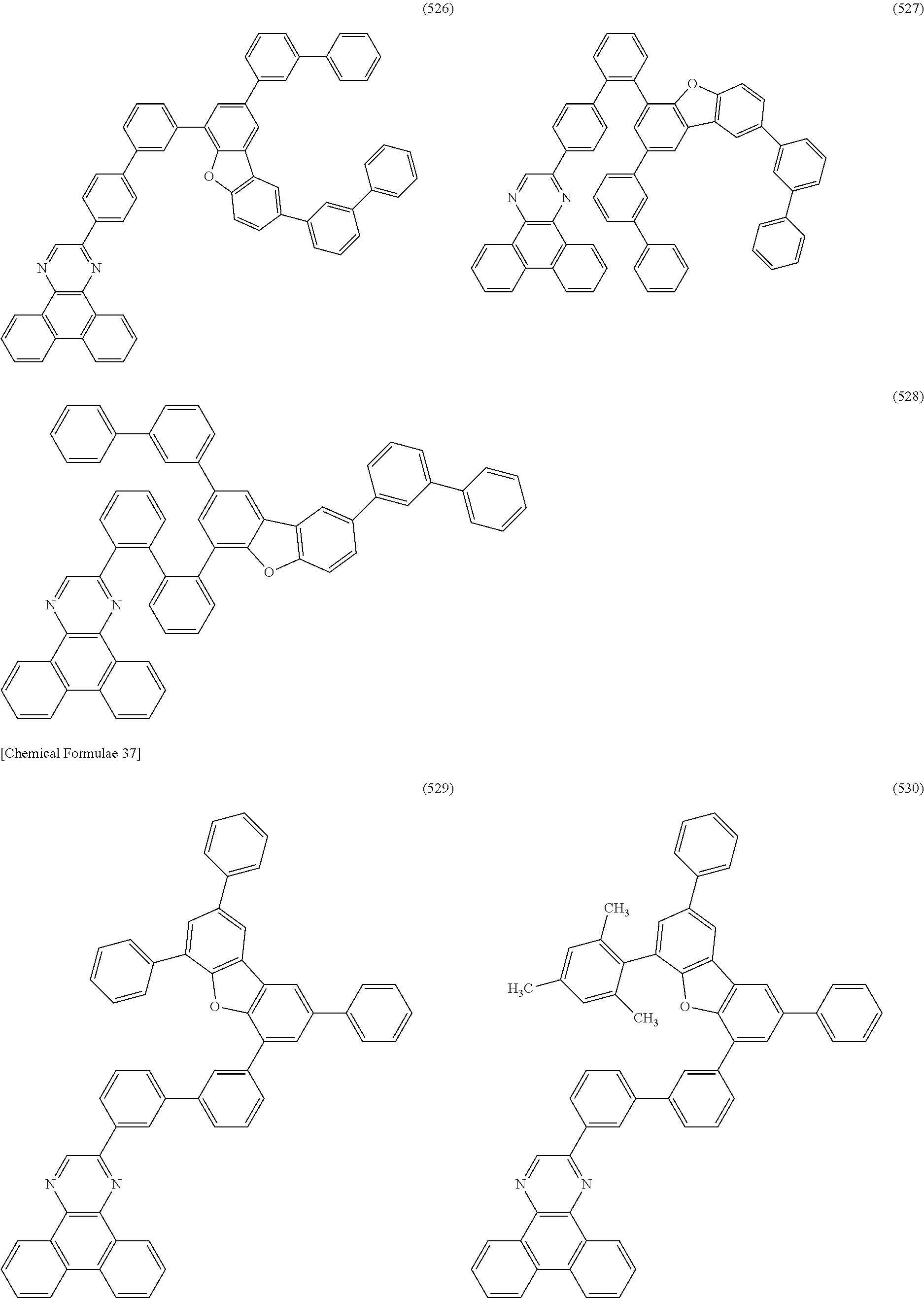

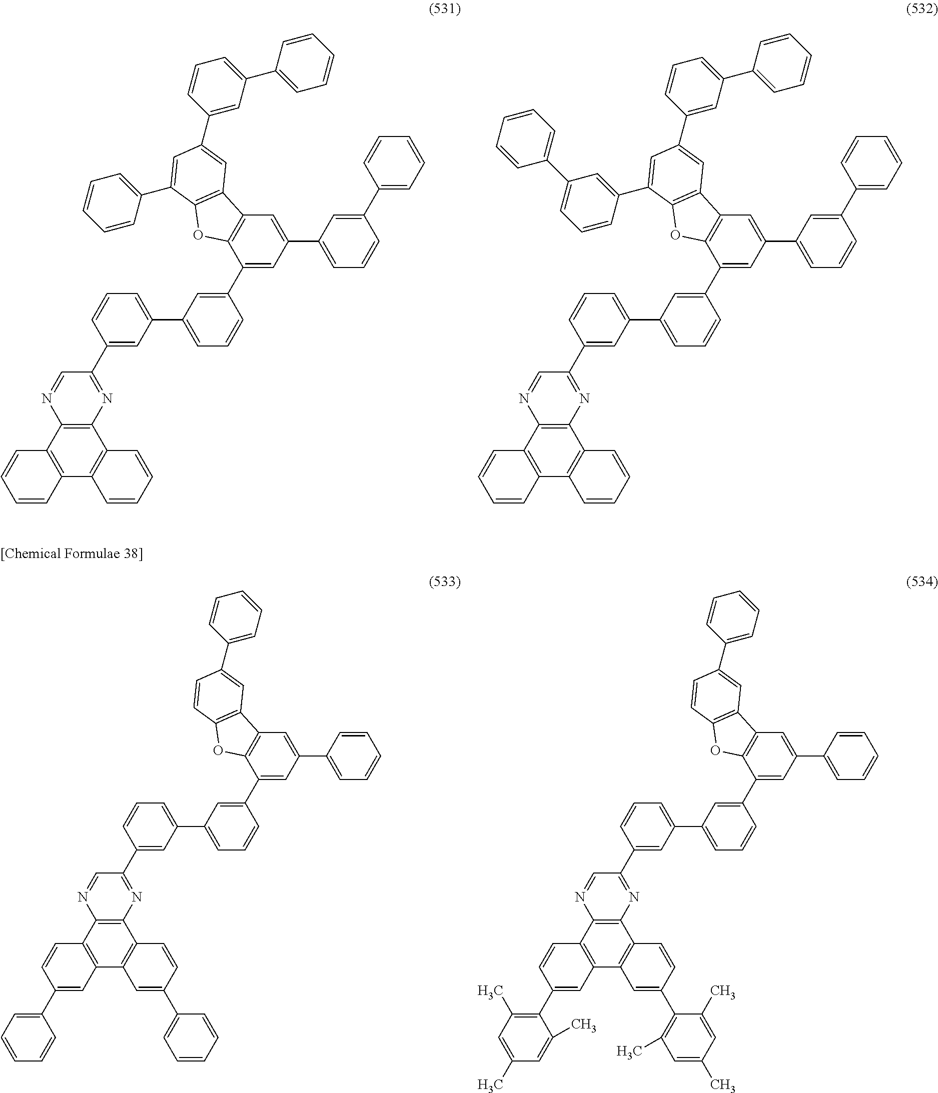

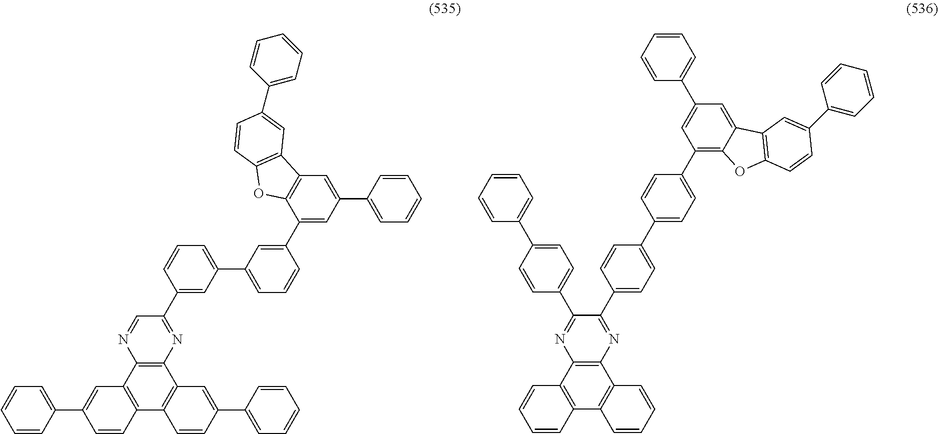

A variety of reactions can be applied to a method of synthesizing the heterocyclic compound of an embodiment of the present invention. For example, synthesis reactions described below enable the synthesis of the heterocyclic compound of an embodiment of the present invention represented by the general formula (G1) or (G2). Note that the method of synthesizing the heterocyclic compound which is an embodiment of the present invention is not limited to the synthesis methods below.

### Method 1 of Synthesizing Heterocyclic Compound Represented by General Formula (G1) or (G2)

First, a synthesis scheme (A-1) is illustrated below.

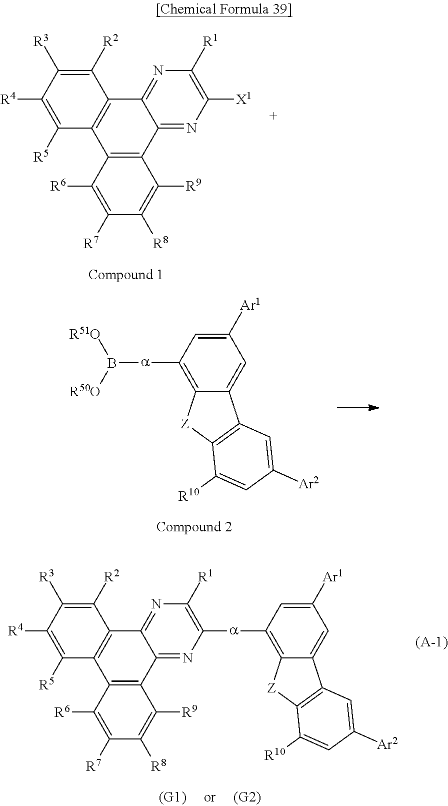

The heterocyclic compound (G1) or (G2) of an embodiment of the present invention can be synthesized as illustrated in the synthesis scheme (A-1). Specifically, a halide of a dibenzo[f,h]quinoxaline derivative (compound 1) is coupled with boronic acid or an organoboron compound of a dibenzofuran derivative or a dibenzothiophene derivative (compound 2) by the Suzuki-Miyaura reaction, whereby the heterocyclic compound (G1) or (G2) described in this embodiment can be obtained.

In the synthesis scheme (A-1), α represents a substituted or unsubstituted phenylene group or a substituted or unsubstituted biphenyldiyl group, Ar¹ and Ar² each represent a substituted or unsubstituted phenyl group or a substituted or unsubstituted biphenyl group, R¹ to R¹⁰ independently represent hydrogen, an alkyl group having 1 to 4 carbon atoms, or a substituted or unsubstituted aryl group having 6 to 13 carbon atoms, and R⁵⁰ and R⁵¹ independently represent hydrogen or an alkyl group having 1 to 6 carbon atoms. In the synthesis scheme (A-1), R⁵⁰ and R⁵¹ may be bonded to each other to form a ring. Further, X¹ represents a halogen.

Examples of a palladium catalyst which can be used in the synthesis scheme (A-1) include, but are not limited to, palladium(II)acetate, tetrakis(triphenylphosphine)palladium(0), bis(triphenylphosphine)palladium(II)dichloride, and the like.

Examples of a ligand of the palladium catalyst which can be used in the synthesis scheme (A-1) include, but are not limited to, tri(ortho-tolyl)phosphine, triphenylphosphine, tricyclohexylphosphine, and the like.

Examples of a base which can be used in the synthesis scheme (A-1) include, but are not limited to, an organic base such as sodium tert-butoxide, an inorganic base such as potassium carbonate or sodium carbonate, and the like.

Examples of a solvent which can be used in the synthesis scheme (A-1) include, but are not limited to, a mixed solvent of toluene and water; a mixed solvent of toluene, alcohol such as ethanol, and water; a mixed solvent of xylene and water; a mixed solvent of xylene, alcohol such as ethanol, and water; a mixed solvent of benzene and water; a mixed solvent of benzene, alcohol such as ethanol, and water; a mixed solvent of water and an ether such as ethylene glycol dimethyl ether; and the like. It is more preferable to use a mixed solvent of toluene and water, a mixed solvent of toluene, ethanol, and water, or a mixed solvent of water and an ether such as ethylene glycol dimethyl ether.

As the coupling reaction illustrated in the synthesis scheme (A-1), the Suzuki-Miyaura reaction using the organoboron compound or the boronic acid represented by the compound 2 may be replaced with a cross coupling reaction using an organoaluminum compound, an organozirconium compound, an organozinc compound, an organotin compound, or the like. However, the present invention is not limited thereto.

Further, in the Suzuki-Miyaura coupling reaction illustrated in the synthesis scheme (A-1), an organoboron compound or boronic acid of a dibenzo[f,h]quinoxaline derivative may be coupled with a halide of a carbazole derivative, a dibenzofuran derivative, or a dibenzothiophene derivative or with a carbazole derivative, a dibenzofuran derivative, or a dibenzothiophene derivative which has a triflate group as a substituent, by the Suzuki-Miyaura reaction.

Thus, the heterocyclic compound of this embodiment can be synthesized.

### Method 2 of Synthesizing Heterocyclic Compound Represented by General Formula (G1) or (G2)

Another method of synthesizing the heterocyclic compound represented by the general formula (G1) or (G2) will be described below. First, a synthesis scheme (B-1) in which a boron compound is used as a material is illustrated below.

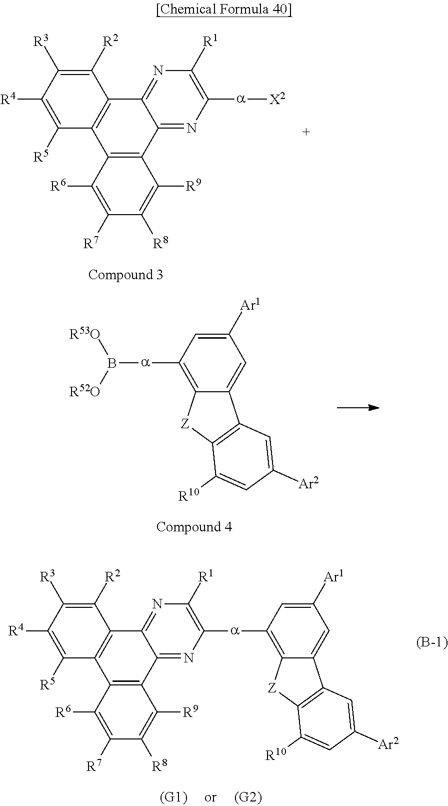

As illustrated in the synthesis scheme (B-1), a halide of a dibenzo[f,h]quinoxaline derivative (compound 3) is coupled with an organoboron compound or boronic acid of a dibenzofuran derivative or a dibenzothiophene derivative (compound 4) by the Suzuki-Miyaura reaction, whereby the heterocyclic compound (G1) or (G2) described in this embodiment can be obtained.

In the synthesis scheme (B-1), α represents a substituted or unsubstituted phenylene group or a substituted or unsubstituted biphenyldiyl group, Ar¹ and Ar² each represent a substituted or unsubstituted phenyl group or a substituted or unsubstituted biphenyl group, R¹ to R¹⁰ independently represent hydrogen, an alkyl group having 1 to 4 carbon atoms, or a substituted or unsubstituted aryl group having 6 to 13 carbon atoms, and R⁵² and R⁵³ each represent hydrogen or an alkyl group having 1 to 6 carbon atoms. In the synthesis scheme (B-1), R⁵² and R⁵³ may be bonded to each other to form a ring. Further, X² represents a halogen or a triflate group, and the halogen is preferably iodine or bromine.

Examples of a palladium catalyst which can be used in the synthesis scheme (B-1) include, but are not limited to, palladium(II)acetate, tetrakis(triphenylphosphine)palladium(0), bis(triphenylphosphine)palladium(II)dichloride, and the like.

Examples of a ligand of the palladium catalyst which can be used in the synthesis scheme (B-1) include, but are not limited to, tri(ortho-tolyl)phosphine, triphenylphosphine, tricyclohexylphosphine, and the like.

Examples of a base which can be used in the synthesis scheme (B-1) include, but are not limited to, an organic base such as sodium tert-butoxide, an inorganic base such as potassium carbonate or sodium carbonate, and the like.

Examples of a solvent which can be used in the synthesis scheme (B-1) include, but are not limited to, a mixed solvent of toluene and water; a mixed solvent of toluene, alcohol such as ethanol, and water; a mixed solvent of xylene and water; a mixed solvent of xylene, alcohol such as ethanol, and water; a mixed solvent of benzene and water; a mixed solvent of benzene, alcohol such as ethanol, and water; a mixed solvent of water and an ether such as ethylene glycol dimethyl ether; and the like. It is more preferable to use a mixed solvent of toluene and water, a mixed solvent of toluene, ethanol, and water, or a mixed solvent of water and an ether such as ethylene glycol dimethyl ether.

As the coupling reaction illustrated in the synthesis scheme (B-1), the Suzuki-Miyaura reaction using the organoboron compound or the boronic acid represented by the compound 4 may be replaced with a cross coupling reaction using an organoaluminum compound, an organozirconium compound, an organozinc compound, an organotin compound, or the like. However, the present invention is not limited thereto. Further, in this coupling, a triflate group or the like may be used other than a halogen; however, the present invention is not limited thereto.

Further, in the Suzuki-Miyaura coupling reaction illustrated in the synthesis scheme (B-1), an organoboron compound or boronic acid of a dibenzo[f,h]quinoxaline derivative may be coupled with a halide of a carbazole derivative, a dibenzofuran derivative, or a dibenzothiophene derivative or with a carbazole derivative, a dibenzofuran derivative, or a dibenzothiophene derivative which has a triflate group as a substituent, by the Suzuki-Miyaura reaction.

Thus, the heterocyclic compound of this embodiment can be synthesized.

Since the heterocyclic compound of this embodiment has excellent heat resistance, a light-emitting element can have a long lifetime. Further, the heterocyclic compound of this embodiment has a wide band gap; accordingly, by use of the heterocyclic compound as a host material in which a light-emitting substance of a light-emitting layer is dispersed in a light-emitting element, high current efficiency can be obtained. In particular, the heterocyclic compound of this embodiment is suitably used as a host material in which a phosphorescent compound is dispersed. Further, since the heterocyclic compound of this embodiment is a substance having a high electron-transport property, it can be suitably used as a material for an electron-transport layer in a light-emitting element. By the use of the heterocyclic compound of this embodiment, a light-emitting element having a low driving voltage can be obtained.

In addition, a light-emitting element having high current efficiency can be obtained. Furthermore, by the use of this light-emitting element, a light-emitting device, an electronic device, and a lighting device in which power consumption is reduced can be obtained.

### Embodiment 2

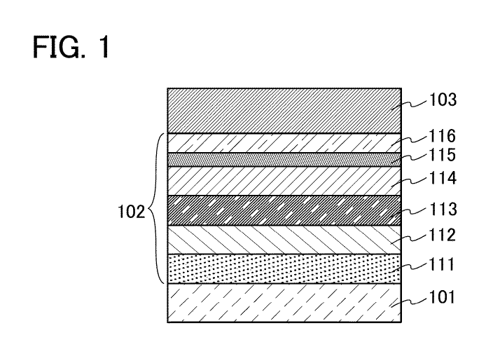

In this embodiment, as one mode of the present invention, a light-emitting element in which any of the heterocyclic compounds described in Embodiment 1 can be used will be described with reference to FIG. 1.

In a light-emitting element described in this embodiment, as illustrated in FIG. 1, an EL layer 102 including a light-emitting layer 113 is provided between a pair of electrodes (a first electrode (anode) 101 and a second electrode (cathode) 103), and the EL layer 102 includes a hole-injection layer 111, a hole-transport layer 112, an electron-transport layer 114, an electron-injection layer 115, a charge-generation layer (E) 116, and the like in addition to the light-emitting layer 113.

By application of a voltage to such a light-emitting element, holes injected from the first electrode 101 side and electrons injected from the second electrode 103 side recombine in the light-emitting layer 113 to raise a substance contained in the light-emitting layer 113 to an excited state. Then, light is emitted when the substance in the excited state returns to the ground state.

The hole-injection layer 111 included in the EL layer 102 is a layer containing a substance having a high hole-transport property and an acceptor substance. When electrons are extracted from the substance having a high hole-transport property owing to the acceptor substance, holes are generated. Thus, holes are injected from the hole-injection layer 111 into the light-emitting layer 113 through the hole-transport layer 112.

The charge-generation layer (E) 116 is a layer containing a substance having a high hole-transport property and an acceptor substance. Electrons are extracted from the substance having a high hole-transport property owing to the acceptor substance, and the extracted electrons are injected from the electron-injection layer 115 having an electron-injection property into the light-emitting layer 113 through the electron-transport layer 114.

A specific example of the light-emitting element described in this embodiment is described below.

For the first electrode (anode) 101 and the second electrode (cathode) 103, a metal, an alloy, an electrically conductive compound, a mixture thereof, and the like can be used. Specifically, indium oxide-tin oxide (ITO: indium tin oxide), indium oxide-tin oxide containing silicon or silicon oxide, indium oxide-zinc oxide (indium zinc oxide), indium oxide containing tungsten oxide and zinc oxide, gold (Au), platinum (Pt), nickel (Ni), tungsten (W), chromium (Cr), molybdenum (Mo), iron (Fe), cobalt (Co), copper (Cu), palladium (Pd), and titanium (Ti) can be used. In addition, an element belonging to Group 1 or Group 2 of the periodic table, for example, an alkali metal such as lithium (Li) or cesium (Cs), an alkaline earth metal such as calcium (Ca) or strontium (Sr), magnesium (Mg), or an alloy thereof (e.g., MgAg or AlLi); a rare earth metal such as europium (Eu) or ytterbium (Yb) or an alloy thereof; graphene; and the like can be used. The first electrode (anode) 101 and the second electrode (cathode) 103 can be formed by, for example, a sputtering method, an evaporation method (including a vacuum evaporation method), or the like.

As the substance having a high hole-transport property used for the hole-injection layer 111, the hole-transport layer 112, and the charge-generation layer (E) 116, the following can be given, for example: aromatic amine compounds such as 4,4′-bis[N-(1-naphthyl)-N-phenylamino]biphenyl (abbreviation: NPB or α-NPD), N,N′-bis(3-methylphenyl)-N,N′-diphenyl-[1,1′-biphenyl]-4,4′-diamine (abbreviation: TPD), 4,4′,4″-tris(carbazol-9-yl)triphenylamine (abbreviation: TCTA), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluoren-2-yl)-N-phenylamino]biphenyl (abbreviation: BSPB); 3-[N-(9-phenylcarbazol-3-yl)-N-phenylamino]-9-phenylcarbazole (abbreviation: PCzPCA1); 3,6-bis[N-(9-phenylcarbazol-3-yl)-N-phenylamino]-9-phenylcarbazole (abbreviation: PCzPCA2); 3-[N-(1-naphthyl)-N-(9-phenylcarbazol-3-yl)amino]-9-phenylcarbazole (abbreviation: PCzPCN1); and the like. A carbazole compound, such as 4,4′-di(N-carbazolyl)biphenyl (abbreviation: CBP), 1,3,5-tris[4-(N-carbazolyl)phenyl]benzene (abbreviation: TCPB), or 9-[4-(10-phenyl-9-anthracenyl)phenyl]-9H-carbazole (abbreviation: CzPA), or the like can be used. The substances mentioned here are mainly ones that have a hole mobility of 10⁻⁶ cm²/Vs or more. Further, other than these substances, any substance that has a property of transporting more holes than electrons may be used.

A high molecular compound such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N-phenylamino}phenyl)methacryla mide] (abbreviation: PTPDMA), or poly[N,N′-bis(4-butylphenyl)-N,N-bis(phenyl)benzidine] (abbreviation: Poly-TPD) can also be used.

Note that the heterocyclic compound of an embodiment of the present invention can also be used as the material having a high hole-transport property.

As examples of the acceptor substance that is used for the hole-injection layer 111 and the charge-generation layer (E) 116, a transition metal oxide or an oxide of a metal belonging to any of Group 4 to Group 8 of the periodic table can be given. Specifically, molybdenum oxide is particularly preferable.

The light-emitting layer 113 is a layer containing a light-emitting substance. The light-emitting layer 113 may contain only a light-emitting substance; alternatively, an emission center substance may be dispersed in a host material in the light-emitting layer 113.

There is no particular limitation on materials that can be used as the light-emitting substance and the emission center substance in the light-emitting layer 113, and light emitted from these substances may be either fluorescence or phosphorescence. Examples of the above light-emitting substance and the emission center substance include the following substances.

Examples of the substance which emits fluorescence include N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra(tert-butyl)perylene (abbreviation: TBP), 4-(10-phenyl-9-anthryl)-4′-(9-phenyl-9H-carbazol-3-yl)triphenylamine (abbreviation: PCBAPA), N,N″-(2-tert-butylanthracene-9,10-diyldi-4,1-phenylene)bis[N,N′,N′-triphenyl-1,4-phenylenediamine] (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N′,N′-triphenyl-1,4-phenylenediamine (abbreviation: 2DPAPPA), N,N,N′,N′,N″,N″,N′″,N′″-octaphenyldibenzo[g,p]chrysene-2,7,10,15-tetraamine (abbreviation: DBC1), coumarin 30, N-(9,10-diphenyl-2-anthryl)-N,9-diphenyl-9H-carbazol-3-amine (abbreviation: 2PCAPA), N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), 9,10-bis(1,1′-biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(1,1′-biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinit rile (abbreviation: DCM1), 2-{2-methyl-6-[2-(2,3,6,7-tetrahydro-1H,5H-benzo[ij]quinolizin-9-yl)ethenyl]-4H-pyra n-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-α]fluoranthene-3,10-d iamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis {2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinoli zin-9-yl)ethenyl]-4H-pyran-4-ylidene}propanedinitrile (abbreviation: BisDCJTM), and the like.

Note that as the substance which emits fluorescence, it is also possible to use the heterocyclic compound of an embodiment of the present invention.

Examples of the substance which emits phosphorescence include bis[2-(3′,5′-bistrifluoromethylphenyl)pyridinato-N,C^{2′}]iridium(III) picolinate (abbreviation: Ir(CF₃ppy)₂(pic)), bis[2-(4′,6′-difluorophenyl)pyridinato-N,C^{2′}]iridium(III) acetylacetonate (abbreviation: FIracac), tris(2-phenylpyridinato)iridium(III) (abbreviation: Ir(ppy)₃), bis(2-phenylpyridinato)iridium(III) acetylacetonate (abbreviation: Ir(ppy)₂(acac)), tris(acetylacetonato)(monophenanthroline)terbium(III) (abbreviation: Tb(acac)₃(Phen)), bis(benzo[h]quinolinato)iridium(III) acetylacetonate (abbreviation: Ir(bzq)₂(acac)), bis(2,4-diphenyl-1,3-oxazolato-N,C^{2′})iridium(III) acetylacetonate (abbreviation: Ir(dpo)₂(acac)), bis{2-[4′-(perfluorophenyl)phenyl]pyridinato-N,C^{2′}}iridium(III) acetylacetonate (abbreviation: Ir(p-PF-ph)₂(acac)), bis(2-phenylbenzothiazolato-N,C^{2′})iridium(III) acetylacetonate (abbreviation: Ir(bt)₂(acac)), bis[2-(2′-benzo[4,5-a]thienyl)pyridinato-N,C^{3′}]iridium(III) acetylacetonate (abbreviation: Ir(btp)₂(acac)), bis(1-phenylisoquinolinato-N,C^{2′})iridium(III) acetylacetonate (abbreviation: Ir(piq)₂(acac)), (acetylacetonato)bis[2,3-bis(4-fluorophenyl)quinoxalinato]iridium(III) (abbreviation: Ir(Fdpq)₂(acac)), (acetylacetonato)bis(3,5-dimethyl-2-phenylpyrazinato)iridium(III) (abbreviation [Ir(mppr-Me)₂(acac)]), (acetylacetonato)bis(5-isopropyl-3-methyl-2-phenylpyrazinato)iridium(III) (abbreviation: [Ir(mppr-iPr)₂(acac)]), (acetylacetonato)bis(2,3,5-triphenylpyrazinato)iridium(III) (abbreviation: Ir(tppr)₂(acac)), bis(2,3,5-triphenylpyrazinato)(dipivaloylmethanato)iridium(III) (abbreviation: [Ir(tppr)₂(dpm)], (acetylacetonato)bis(6-tert-butyl-4-phenylpyrimidinato)iridium(III) (abbreviation: [Ir(tBuppm)₂(acac)]), (acetylacetonato)bis(4,6-diphenylpyrimidinato)iridium(III) (abbreviation: [Ir(dppm)₂(acac)], 2,3,7,8,12,13,17,18-octaethyl-21H,23H-porphyrin platinum(II) (abbreviation: PtOEP), tris(1,3-diphenyl-1,3-propanedionato)(monophenanthroline)europium(III) (abbreviation: Eu(DBM)₃(Phen)), tris[1-(2-thenoyl)-3,3,3-trifluoroacetonato](monophenanthroline)europium(III) (abbreviation: Eu(TTA)₃(Phen)), and the like.

Although there is no particular limitation on a material that can be used as the host material described above, any of the following substances can be used for the host material, for example: metal complexes such as tris(8-quinolinolato)aluminum(III) (abbreviation: Alq), tris(4-methyl-8-quinolinolato)aluminum(III) (abbreviation: Almq₃), bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), and bis[2-(2-benzothiazolyl)phenolato]zinc(II) (abbreviation: ZnBTZ); heterocyclic compounds such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 3-(4-biphenylyl)-4-phenyl-5-(4-tert-butylphenyl)-1,2,4-triazole (abbreviation: TAZ), 2,2′,2″-(1,3,5-benzenetriyl)-tris(1-phenyl-1H-benzimidazole) (abbreviation: TPBI), bathophenanthroline (abbreviation: BPhen), bathocuproine (abbreviation: BCP), and 9-[4-(5-phenyl-1,3,4-oxadiazol-2-yl)phenyl]-9H-carbazole (abbreviation: CO11); and aromatic amine compounds such as 4,4′-bis[N-(1-naphthyl)-N-phenylamino]biphenyl (abbreviation: NPB or α-NPD), N,N′-bis(3-methylphenyl)-N,N′-diphenyl-[1,1′-biphenyl]-4,4′-diamine (abbreviation: TPD), and 4,4′-bis[N-(spiro-9,9′-bifluoren-2-yl)-N-phenylamino]biphenyl (abbreviation: BSPB). In addition, condensed polycyclic aromatic compounds such as anthracene derivatives, phenanthrene derivatives, pyrene derivatives, chrysene derivatives, and dibenzo[g,p]chrysene derivatives can be given. Specific examples of the condensed polycyclic aromatic compound include 9,10-diphenylanthracene (abbreviation: DPAnth), N,N-diphenyl-9-[4-(10-phenyl-9-anthryl)phenyl]-9H-carbazol-3-amine (abbreviation: CzAlPA), 4-(10-phenyl-9-anthryl)triphenylamine (abbreviation: DPhPA), 4-(9H-carbazol-9-yl)-4′-(10-phenyl-9-anthryl)triphenylamine (abbreviation: YGAPA), N,9-diphenyl-N-[4-(10-phenyl-9-anthryl)phenyl]-9H-carbazol-3-amine (abbreviation: PCAPA), N,9-diphenyl-N-{4-[4-(10-phenyl-9-anthryl)phenyl]phenyl}-9H-carbazol-3-amine (abbreviation: PCAPBA), N,9-diphenyl-N-(9,10-diphenyl-2-anthryl)-9H-carbazol-3-amine (abbreviation: 2PCAPA), 6,12-dimethoxy-5,11-diphenylchrysene, N,N,N′,N′,N″,N″, N′″,N′″-octaphenyldibenzo[g,p]chrysene-2,7,10,15-tetraamine (abbreviation: DBC1), 9-[4-(10-phenyl-9-anthryl)phenyl]-9H-carbazole (abbreviation: CzPA), 3,6-diphenyl-9-[4-(10-phenyl-9-anthryl)phenyl]-9H-carbazole (abbreviation: DPCzPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), and 1,3,5-tri(1-pyrenyl)benzene (abbreviation: TPB3). One or more substances having a wider energy gap than the emission center substance described above is preferably selected from these substances and known substances. Moreover, in the case where the emission center substance emits phosphorescence, a substance having triplet excitation energy (energy difference between a ground state and a triplet excited state) which is higher than that of the emission center substance is preferably selected as the host material.

Note that as the material that can be used as the above host material, it is also possible to use the heterocyclic compound of an embodiment of the present invention. Since the heterocyclic compound of an embodiment of the present invention has a high S1 level, when the heterocyclic compound is used as a host material for a substance emitting fluorescence, the substance can be used so as to emit light in the visible region.

Note that in the heterocyclic compound of an embodiment of the present invention, in which a dibenzo[f,h]quinoxaline ring and a hole-transport skeleton are bonded through a benzene ring, a dibenzo[f,h]quinoxaline skeleton is considered as a skeleton which predominantly determines the LUMO level. Further, the compound has a deep LUMO level of at least −2.8 eV or less, specifically −2.9 eV or less on the basis of cyclic voltammetry (CV) measurements. For example, the LUMO level of 2mDBTPDBq-II is found to be −2.96 eV by CV measurements. Further, the LUMO level of the phosphorescent compound having a diazine skeleton, typified by the phosphorescent compound having a pyrazine skeleton, such as [Ir(mppr-Me)₂(acac)], [Ir(mppr-iPr)₂(acac)], [Ir(tppr)₂(acac)], or [Ir(tppr)₂(dpm)], or the phosphorescent compound having a pyrimidine skeleton, such as [Ir(tBuppm)₂(acac)] or [Ir(dppm)₂(acac)] is substantially as deep as the LUMO level of the heterocyclic compound of an embodiment of the present invention. Therefore, when a light-emitting layer includes the heterocyclic compound of an embodiment of the present invention as a host material and a phosphorescent compound having a diazine skeleton (particularly a pyrazine skeleton or a pyrimidine skeleton) as a guest material, traps for electrons in the light-emitting layer can be reduced to a minimum, and extremely low driving voltage can be achieved.

Note that the light-emitting layer 113 may have a structure in which two or more layers are stacked. For example, in the case where the light-emitting layer 113 is formed by stacking a first light-emitting layer and a second light-emitting layer in that order over the hole-transport layer, a substance having a hole-transport property is used for the host material of the first light-emitting layer and a substance having an electron-transport property is used for the host material of the second light-emitting layer.

The electron-transport layer 114 is a layer containing a substance having a high electron-transport property. For the electron-transport layer 114, it is possible to use a metal complex such as Alq₃, tris(4-methyl-8-quinolinolato)aluminum (abbreviation: Almq₃), bis(10-hydroxybenzo[h]quinolinato)beryllium (abbreviation: BeBq₂), BAlq, Zn(BOX)₂, or bis[2-(2-hydroxyphenyl)benzothiazolato]zinc (abbreviation: Zn(BTZ)₂). It is also possible to use a heteroaromatic compound such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), or 4,4′-bis(5-methylbenzoxazol-2-yl)stilbene (abbreviation: BzOs). It is also possible to use a high molecular compound such as poly(2,5-pyridinediyl) (abbreviation: PPy), poly[(9,9-dihexylfluorene-2,7-diyl)-co-(pyridine-3,5-diyl)] (abbreviation: PF-Py), or poly[(9,9-dioctylfluorene-2,7-diyl)-co-(2,2′-bipyridine-6,6′-diyl)] (abbreviation: PF-BPy). The substances mentioned here mainly have an electron mobility of 10⁻⁶ cm²/Vs or more. Note that other than these substances, a substance that has a property of transporting more electrons than holes may be used for the electron-transport layer.

Note that the heterocyclic compound of an embodiment of the present invention can also be used as the substance having a high electron-transport property.

Furthermore, the electron-transport layer is not limited to a single layer and may be a stack of two or more layers containing any of the above substances.

The electron-injection layer 115 is a layer containing a substance having a high electron-injection property. For the electron-injection layer 115, an alkali metal, an alkaline earth metal, or a compound thereof such as lithium fluoride (LiF), cesium fluoride (CsF), calcium fluoride (CaF₂), or lithium oxide (LiOₓ) can be used. Alternatively, a rare earth metal compound such as erbium fluoride (ErF₃) can be used. Any of the above substances for forming the electron-transport layer 114 can also be used.

Alternatively, a composite material in which an organic compound and an electron donor (donor) are mixed may be used for the electron-injection layer 115. The electron donor causes electron generation in the organic compound, and thus such a composite material is excellent in an electron-injection property and an electron-transport property. The organic compound here is preferably a material excellent in transporting the generated electrons; specific examples include the above-described substances for forming the electron-transport layer 114 (e.g., a metal complex or a heteroaromatic compound). The electron donor is preferably a substance showing an electron-donating property with respect to the organic compound. Specifically, an alkali metal, an alkaline earth metal, and a rare earth metal are preferable, and lithium, cesium, magnesium, calcium, erbium, ytterbium, and the like can be given, for example. An alkali metal oxide and an alkaline earth metal oxide are also preferable and examples include lithium oxide, calcium oxide, barium oxide, and the like. A Lewis base such as magnesium oxide can also be used. An organic compound such as tetrathiafulvalene (abbreviation: TTF) can also be used.

Note that each of the above-described hole-injection layer 111, hole-transport layer 112, light-emitting layer 113, electron-transport layer 114, electron-injection layer 115, and charge-generation layer (E) 116 can be formed by a method such as an evaporation method (e.g., a vacuum evaporation method), an inkjet method, or a coating method.

In the above-described light-emitting element, current flows due to a potential difference generated between the first electrode 101 and the second electrode 103 and holes and electrons recombine in the EL layer 102, whereby light is emitted. Then, the emitted light is extracted outside through one or both of the first electrode 101 and the second electrode 103. Therefore, one or both of the first electrode 101 and the second electrode 103 are electrodes having a light-transmitting property.

The above-described light-emitting element is formed using the heterocyclic compound of an embodiment of the present invention, whereby not only the heat resistance but also the element efficiency of the light-emitting element can be improved and an increase in driving voltage can be minimized.

Although the heterocyclic compound of an embodiment of the present invention is more preferably used as a host material for a phosphorescent substance of a light-emitting layer, the heterocyclic compound of an embodiment of the present invention can also be used as a light-emitting substance in a light-emitting layer or a host material for a fluorescent substance in a light-emitting layer, or for another layer (e.g., a hole-injection layer, a hole-transport layer, or an electron-transport layer). Therefore, a common material can be used for a plurality of layers. Accordingly, cost of synthesis of a material and manufacture of a light-emitting element can be reduced, which is preferable.

Note that the light-emitting element described in this embodiment is an example of a light-emitting element manufactured using the heterocyclic compound of an embodiment of the present invention. Further, as a light-emitting device including the above light-emitting element, a passive matrix light-emitting device and an active matrix light-emitting device can be manufactured. It is also possible to manufacture a light-emitting device with a microcavity structure including a light-emitting element having a structure different from that of a light-emitting element described in another embodiment. All of the above light-emitting devices are included in the present invention. Note that the power consumption of these light-emitting devices can be reduced.

Note that there is no particular limitation on a structure of a TFT in the case of manufacturing the active matrix light-emitting device. For example, a staggered TFT or an inverted staggered TFT can be used as appropriate. Further, a driver circuit formed over a TFT substrate may be formed using both an n-type TFT and a p-type TFT or only either an n-type TFT or a p-type TFT. Furthermore, there is also no particular limitation on crystallinity of a semiconductor film used for the TFT. For example, an amorphous semiconductor film, a crystalline semiconductor film, an oxide semiconductor film, or the like can be used.

Note that the structure described in this embodiment can be combined as appropriate with any of the structures described in the other embodiments.

### Embodiment 3

In this embodiment, as one mode of the present invention, a light-emitting element in which two or more kinds of organic compounds as well as a phosphorescent compound are used for a light-emitting layer will be described.

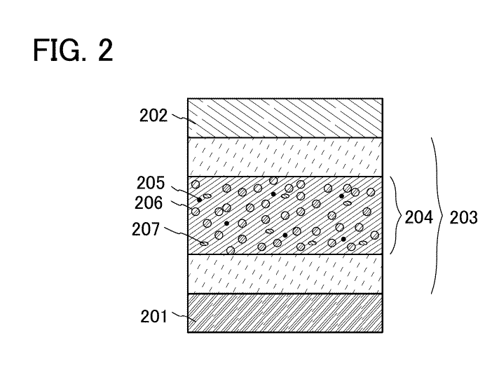

A light-emitting element described in this embodiment includes an EL layer 203 between a pair of electrodes (an anode 201 and a cathode 202) as illustrated in FIG. 2. Note that the EL layer 203 includes at least a light-emitting layer 204 and may include a hole-injection layer, a hole-transport layer, an electron-transport layer, an electron-injection layer, a charge-generation layer (E), and the like. Note that for the hole-injection layer, the hole-transport layer, the electron-transport layer, the electron-injection layer, and the charge-generation layer (E), the substances described in Embodiment 2 can be used.

The light-emitting layer 204 described in this embodiment contains a phosphorescent compound 205, a first organic compound 206, and a second organic compound 207. The heterocyclic compound described in Embodiment 1 can be used as the first organic compound 206 or the second organic compound 207. Note that the phosphorescent compound 205 is a guest material in the light-emitting layer 204. Moreover, one of the first organic compound 206 and the second organic compound 207, the content of which is higher than that of the other in the light-emitting layer 204, is a host material in the light-emitting layer 204.

When the light-emitting layer 204 has the structure in which the guest material is dispersed in the host material, crystallization of the light-emitting layer can be suppressed. Further, it is possible to suppress concentration quenching due to high concentration of the guest material, and thus the light-emitting element can have higher emission efficiency.

Note that it is preferable that a triplet excitation energy level (T1 level) of each of the first organic compound 206 and the second organic compound 207 be higher than that of the phosphorescent compound 205. This is because, when the T1 level of the first organic compound 206 (or the second organic compound 207) is lower than that of the phosphorescent compound 205, the triplet excitation energy of the phosphorescent compound 205, which is to contribute to light emission, is quenched by the first organic compound 206 (or the second organic compound 207) and accordingly the emission efficiency is decreased.

Here, for improvement in the efficiency of energy transfer from a host material to a guest material, Forster mechanism (dipole-dipole interaction) and Dexter mechanism (electron exchange interaction), which are known as mechanisms of energy transfer between molecules, are considered. According to the mechanisms, it is preferable that an emission spectrum of a host material (a fluorescence spectrum in the case of energy transfer from a singlet excited state, and a phosphorescence spectrum in the case of energy transfer from a triplet excited state) largely overlap with an absorption spectrum of a guest material (specifically, the spectrum of the longest wavelength (lowest energy) absorption band). However, in general, it is difficult to obtain an overlap between a fluorescence spectrum of a host material and the absorption spectrum of the longest wavelength (lowest energy) absorption band of a guest material.

The reason for this is as follows: if the fluorescence spectrum of the host material overlaps with the absorption spectrum of the longest wavelength (lowest energy) absorption band of the guest material, since a phosphorescence spectrum of the host material is located on a longer wavelength (lower energy) side as compared to the fluorescence spectrum, the T1 level of the host material becomes lower than the T1 level of the phosphorescent compound and the above-described problem of quenching occurs; yet, when the host material is designed in such a manner that the T1 level of the host material is higher than the T1 level of the phosphorescent compound to avoid the problem of quenching, the fluorescence spectrum of the host material is shifted to the shorter wavelength (higher energy) side, and thus the fluorescence spectrum does not have any overlap with the absorption spectrum of the longest wavelength (lowest energy) absorption band of the guest material. For that reason, in general, it is difficult to obtain an overlap between a fluorescence spectrum of a host material and an absorption spectrum in an absorption band on the longest wavelength (lowest energy) side of a guest material so as to maximize energy transfer from a singlet excited state of the host material.

Thus, in this embodiment, a combination of the first organic compound and the second organic compound preferably forms an exciplex. In that case, the first organic compound 206 and the second organic compound 207 form an exciplex (also referred to as excited complex) at the time of recombination of carriers (electrons and holes) in the light-emitting layer 204. Thus, in the light-emitting layer 204, a fluorescence spectrum of the first organic compound 206 and that of the second organic compound 207 are converted into an emission spectrum of the exciplex which is located on a longer wavelength side. Moreover, when the first organic compound and the second organic compound are selected in such a manner that the emission spectrum of the exciplex largely overlaps with the absorption spectrum of the guest material, energy transfer from a singlet excited state can be maximized. Note that also in the case of a triplet excited state, energy transfer from the exciplex, not the host material, is assumed to occur.

For the phosphorescent compound 205, a phosphorescent organometallic complex is preferably used. Although the combination of the first organic compound 206 and the second organic compound 207 can be determined such that an exciplex is formed, a combination of a compound which is likely to accept electrons (a compound having an electron-trapping property) and a compound which is likely to accept holes (a compound having a hole-trapping property) is preferably employed.

Examples of a phosphorescent organometallic complex include bis[2-(3′,5′-bistrifluoromethylphenyl)pyridinato-N,C^{2′}]iridium(III) picolinate (abbreviation: Ir(CF₃ppy)₂(pic)), bis[2-(4′,6′-difluorophenyl)pyridinato-N,C^{2′}]iridium(III) acetylacetonate (abbreviation: FIracac), tris(2-phenylpyridinato)iridium(III) (abbreviation: Ir(ppy)₃), bis(2-phenylpyridinato)iridium(III) acetylacetonate (abbreviation: Ir(ppy)₂(acac)), tris(acetylacetonato)(monophenanthroline)terbium(III) (abbreviation: Tb(acac)₃(Phen)), bis(benzo[h]quinolinato)iridium(III) acetylacetonate (abbreviation: Ir(bzq)₂(acac)), bis(2,4-diphenyl-1,3-oxazolato-N,C^{2′})iridium(III)acetylacetonate (abbreviation: Ir(dpo)₂(acac)), bis {2-[4′-(perfluorophenyl)phenyl]pyridinato-N,C^{2′}}iridium(III) acetylacetonate (abbreviation: Ir(p-PF-ph)₂(acac)), bis(2-phenylbenzothiazolato-N,C^{2′})iridium(III) acetylacetonate (abbreviation: Ir(bt)₂(acac)), bis[2-(2′-benzo[4,5-a]thienyl)pyridinato-N,C^{3′}]iridium(III) acetylacetonate (abbreviation: Ir(btp)₂(acac)), bis(1-phenylisoquinolinato-N,C^{2′})iridium(III) acetylacetonate (abbreviation: Ir(piq)₂(acac)), (acetylacetonato)bis[2,3-bis(4-fluorophenyl)quinoxalinato]iridium(III) (abbreviation: Ir(Fdpq)₂(acac)), (acetylacetonato)bis(2,3,5-triphenylpyrazinato)iridium(III) (abbreviation: Ir(tppr)₂(acac)), 2,3,7,8,12,13,17,18-octaethyl-21H,23H-porphyrin platinum(II) (abbreviation: PtOEP), tris(1,3-diphenyl-1,3-propanedionato)(monophenanthroline)europium(III) (abbreviation: Eu(DBM)₃(Phen)), tris[1-(2-thenoyl)-3,3,3-trifluoroacetonato](monophenanthroline)europium(III) (abbreviation: Eu(TTA)₃(Phen)), and the like.

As a compound which is likely to accept electrons, a π-electron deficient heteroaromatic compound such as a nitrogen-containing heteroaromatic compound is preferable. For example, a quinoxaline derivative and a dibenzoquinoxaline derivative can be given and examples thereof include 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), and the like. Note that the heterocyclic compound of an embodiment of the present invention can be used as a compound which is likely to accept electrons.

As a compound which is likely to accept holes, a π-electron rich heteroaromatic compound (e.g., a carbazole derivative or an indole derivative) or an aromatic amine compound is preferable. For example, the following can be given: 4-phenyl-4′-(9-phenyl-9H-carbazol-3-yl)triphenylamine (abbreviation: PCBA1BP), 4,4′-di(1-naphthyl)-4″-(9-phenyl-9H-carbazol-3-yl)triphenylamine (abbreviation: PCBNBB), 3-[N-(1-naphthyl)-N-(9-phenylcarbazol-3-yl)amino]-9-phenylcarbazole (abbreviation: PCzPCN1), 4,4′,4″-tris[N-(1-naphthyl)-N-phenylamino]triphenylamine (abbreviation: 1′-TNATA), 2,7-bis[N-(4-diphenylaminophenyl)-N-phenylamino]-spiro-9,9′-bifluorene (abbreviation: DPA2SF), N,N′-bis(9-phenylcarbazol-3-yl)-N,N′-diphenylbenzene-1,3-diamine (abbreviation: PCA2B), N-(9,9-dimethyl-2-N′,N′-diphenylamino-9H-fluoren-7-yl)diphenylamine (abbreviation: DPNF), N,N′,N″-triphenyl-N,N′,N″-tris(9-phenylcarbazol-3-yl)benzene-1,3,5-triamine (abbreviation: PCA3B), 2-[N-(9-phenylcarbazol-3-yl)-N-phenylamino]spiro-9,9′-bifluorene (abbreviation: PCASF), 2-[N-(4-diphenylaminophenyl)-N-phenylamino]spiro-9,9′-bifluorene (abbreviation: DPASF), N,N′-bis[4-(carbazol-9-yl)phenyl]-N,N′-diphenyl-9,9-dimethylfluorene-2,7-diamine (abbreviation: YGA2F), 4,4′-bis[N-(3-methylphenyl)-N-phenylamino]biphenyl (abbreviation: TPD), 4,4′-bis[N-(4-diphenylaminophenyl)-N-phenylamino]biphenyl (abbreviation: DPAB), N-(9,9-dimethyl-9H-fluoren-2-yl)-N-{9,9-dimethyl-2-[N-phenyl-N-(9,9-dimethyl-9H-fl uoren-2-yl)amino]-9H-fluoren-7-yl}phenylamine (abbreviation: DFLADFL), 3-[N-(9-phenylcarbazol-3-yl)-N-phenylamino]-9-phenylcarbazole (abbreviation: PCzPCA1), 3-[N-(4-diphenylaminophenyl)-N-phenylamino]-9-phenylcarbazole (abbreviation: PCzDPA1), 3,6-bis[N-(4-diphenylaminophenyl)-N-phenylamino]-9-phenylcarbazole (abbreviation: PCzDPA2), 4,4′-bis(N-{4-[N-(3-methylphenyl)-N-phenylamino]phenyl}-N-phenylamino)biphenyl (abbreviation: DNTPD), 3,6-bis[N-(4-diphenylaminophenyl)-N-(1-naphthyl)amino]-9-phenylcarbazole (abbreviation: PCzTPN2), and 3,6-bis[N-(9-phenylcarbazol-3-yl)-N-phenylamino]-9-phenylcarbazole (abbreviation: PCzPCA2). Note that the heterocyclic compound of an embodiment of the present invention can be used as a compound which is likely to accept holes.

As for the above-described first and second organic compounds 206 and 207, the present invention is not limited to the above examples. The combination is determined so that an exciplex can be formed, the emission spectrum of the exciplex overlaps with the absorption spectrum of the phosphorescent compound 205, and the peak of the emission spectrum of the exciplex can be at a longer wavelength than the peak of the absorption spectrum of the phosphorescent compound 205.

Note that in the case where a compound which is likely to accept electrons and a compound which is likely to accept holes are used for the first organic compound 206 and the second organic compound 207, carrier balance can be controlled by the mixture ratio of the compounds. Specifically, the ratio of the first organic compound to the second organic compound is preferably 1:9 to 9:1.

In the light-emitting element described in this embodiment, energy transfer efficiency can be improved owing to energy transfer utilizing an overlap between an emission spectrum of an exciplex and an absorption spectrum of a phosphorescent compound. Thus, high external quantum efficiency of the light-emitting element can be achieved.

Note that in another structure of the present invention, the light-emitting layer 204 can be formed using a host molecule having a hole-trapping property and a host molecule having an electron-trapping property as the two kinds of organic compounds other than the phosphorescent compound 205 (guest material) so that a phenomenon (guest coupled with complementary hosts: GCCH) occurs in which holes and electrons are introduced to guest molecules existing in the two kinds of host molecules and the guest molecules are brought into an excited state.

At this time, the host molecule having a hole-trapping property and the host molecule having an electron-trapping property can be respectively selected from the above-described compounds which are likely to accept holes and the above-described compounds which are likely to accept electrons.

Note that the light-emitting element described in this embodiment is one structural example of a light-emitting element; it is possible to apply a light-emitting element having another structure, which is described in another embodiment, to a light-emitting device that is an embodiment of the present invention. Further, as a light-emitting device including the above light-emitting element, a passive matrix light-emitting device and an active matrix light-emitting device can be manufactured. It is also possible to manufacture a light-emitting device with a microcavity structure including a light-emitting element having a structure different from that of a light-emitting element described in another embodiment. All of the above light-emitting devices are included in the present invention.

Note that there is no particular limitation on a structure of a TFT in the case of manufacturing the active matrix light-emitting device. For example, a staggered TFT or an inverted staggered TFT can be used as appropriate. Further, a driver circuit formed over a TFT substrate may be formed using both an n-type TFT and a p-type TFT or only either an n-type TFT or a p-type TFT. Furthermore, there is also no particular limitation on crystallinity of a semiconductor film used for the TFT. For example, an amorphous semiconductor film, a crystalline semiconductor film, an oxide semiconductor film, or the like can be used.

Note that the structure described in this embodiment can be combined as appropriate with any of the structures described in the other embodiments.

### Embodiment 4

In this embodiment, as one mode of the present invention, a light-emitting element (hereinafter referred to as a tandem light-emitting element) in which a charge-generation layer is interposed between a plurality of EL layers will be described.

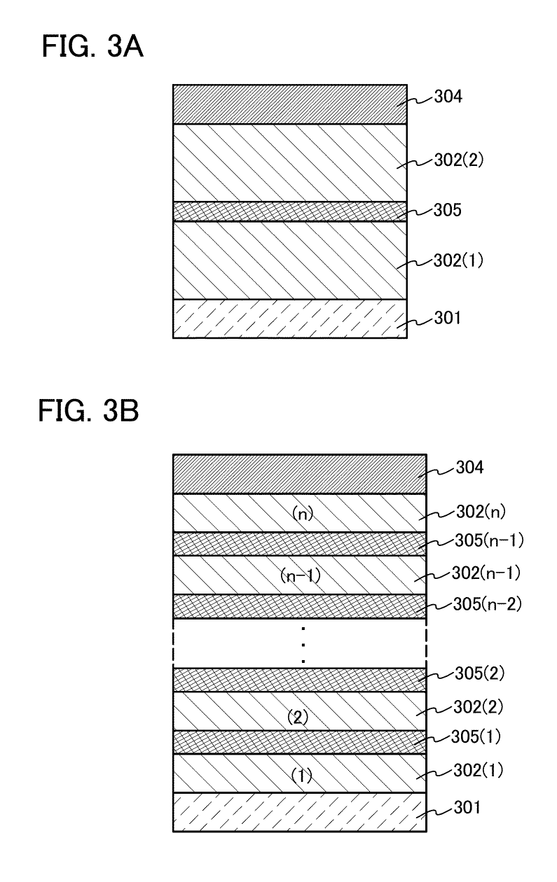

A light-emitting element described in this embodiment is a tandem light-emitting element including a plurality of EL layers (a first EL layer 302(1) and a second EL layer 302(2)) between a pair of electrodes (a first electrode 301 and a second electrode 304) as illustrated in FIG. 3A.

In this embodiment, the first electrode 301 functions as an anode, and the second electrode 304 functions as a cathode. Note that the first electrode 301 and the second electrode 304 can have structures similar to those described in Embodiment 2. In addition, although the plurality of EL layers (the first EL layer 302(1) and the second EL layer 302(2)) may have structures similar to those described in Embodiment 2 or 3, any of the EL layers may have a structure similar to that described in Embodiment 2 or 3. In other words, the structures of the first EL layer 302(1) and the second EL layer 302(2) may be the same or different from each other and can be similar to those described in Embodiment 2 or 3.

Further, a charge-generation layer (1) 305 is provided between the plurality of EL layers (the first EL layer 302(1) and the second EL layer 302(2)). The charge-generation layer (I) 305 has a function of injecting electrons into one of the EL layers and injecting holes into the other of the EL layers when a voltage is applied between the first electrode 301 and the second electrode 304. In this embodiment, when a voltage is applied such that the potential of the first electrode 301 is higher than that of the second electrode 304, the charge-generation layer (I) 305 injects electrons into the first EL layer 302(1) and injects holes into the second EL layer 302(2).

Note that in terms of light extraction efficiency, the charge-generation layer (I) 305 preferably has a light-transmitting property with respect to visible light (specifically, the charge-generation layer (I) 305 has a visible light transmittance of 40% or more). Further, the charge-generation layer (I) 305 functions even if it has lower electric conductivity than the first electrode 301 or the second electrode 304.

The charge-generation layer (I) 305 may have either a structure in which an electron acceptor (acceptor) is added to an organic compound having a high hole-transport property or a structure in which an electron donor (donor) is added to an organic compound having a high electron-transport property. Alternatively, both of these structures may be stacked.

In the case of the structure in which an electron acceptor is added to an organic compound having a high hole-transport property, as the organic compound having a high hole-transport property, for example, an aromatic amine compound such as NPB, TPD, TDATA, MTDATA, or 4,4′-bis[N-(spiro-9,9′-bifluoren-2-yl)-N-phenylamino]biphenyl (abbreviation: BSPB), or the like can be used. The substances mentioned here are mainly ones that have a hole mobility of 10⁻⁶ cm²/Vs or higher. However, another substance may be used as long as the substance is an organic compound having a higher hole-transport property than an electron-transport property. Note that it is also possible to use a heterocyclic compound of an embodiment of the present invention as the organic compound having a high hole-transport property in the charge-generation layer (I) 305.

Further, as the electron acceptor, 7,7,8,8-tetracyano-2,3,5,6-tetrafluoroquinodimethane (abbreviation: F₄-TCNQ), chloranil, or the like can be used. Alternatively, a transition metal oxide can be used. Further alternatively, an oxide of metals that belong to Group 4 to Group 8 of the periodic table can be used. Specifically, it is preferable to use vanadium oxide, niobium oxide, tantalum oxide, chromium oxide, molybdenum oxide, tungsten oxide, manganese oxide, or rhenium oxide because the electron-accepting property is high. Among these, molybdenum oxide is especially preferable because it is stable in the air, has a low hygroscopic property, and is easily handled.

On the other hand, in the case of the structure in which an electron donor is added to an organic compound having a high electron-transport property, as the organic compound having a high electron-transport property, for example, a metal complex having a quinoline skeleton or a benzoquinoline skeleton, such as Alq, Almq₃, BeBq₂, or BAlq, or the like can be used. Alternatively, it is possible to use a metal complex having an oxazole-based ligand or a thiazole-based ligand, such as Zn(BOX)₂ or Zn(BTZ)₂. Further alternatively, instead of a metal complex, it is possible to use PBD, OXD-7, TAZ, BPhen, BCP, or the like. The substances mentioned here are mainly ones that have an electron mobility of 10⁻⁶ cm²/Vs or higher. Note that another substance may be used as long as the substance is an organic compound having a higher electron-transport property than a hole-transport property.

As the electron donor, it is possible to use an alkali metal, an alkaline earth metal, a rare earth metal, a metal belonging to Group 2 or 13 of the periodic table, or an oxide or carbonate thereof. Specifically, it is preferable to use lithium (Li), cesium (Cs), magnesium (Mg), calcium (Ca), ytterbium (Yb), indium (In), lithium oxide, cesium carbonate, or the like. Alternatively, an organic compound such as tetrathianaphthacene may be used as the electron donor.

Note that forming the charge-generation layer (I) 305 by using any of the above materials can suppress an increase in drive voltage caused by the stack of the EL layers.

Although this embodiment shows the light-emitting element having two EL layers, the present invention can be similarly applied to a light-emitting element in which n EL layers (302(1) to 302(n)) (n is three or more) are stacked and charge-generation layers (I) (305(1) to 305(n−1)) are each provided between these EL layers (302(1) to 302(n)) as illustrated in FIG. 3B. In the case where a plurality of EL layers is included between a pair of electrodes as in the light-emitting element according to this embodiment, by provision of a charge-generation layer (I) between the EL layers, light emission in a high luminance region can be obtained with current density kept low. Since the current density can be kept low, the element can have a long lifetime. When the light-emitting element is applied for lighting, voltage drop due to resistance of an electrode material can be reduced, whereby uniform light emission in a large area is possible. Moreover, it is possible to achieve a light-emitting device of low power consumption, which can be driven at a low voltage.

By making the EL layers emit light of different colors from each other, the light-emitting element can provide light emission of a desired color as a whole. For example, by forming a light-emitting element having two EL layers such that the emission color of the first EL layer and the emission color of the second EL layer are complementary colors, the light-emitting element can provide white light emission as a whole. Note that the word “complementary” means color relationship in which an achromatic color is obtained when colors are mixed. In other words, when lights obtained from substances which emit light of complementary colors are mixed, white emission can be obtained.

Further, the same can be applied to a light-emitting element having three EL layers. For example, the light-emitting element as a whole can provide white light emission when the emission color of the first EL layer is red, the emission color of the second EL layer is green, and the emission color of the third EL layer is blue.

Note that the structure described in this embodiment can be combined as appropriate with any of the structures described in the other embodiments.

### Embodiment 5

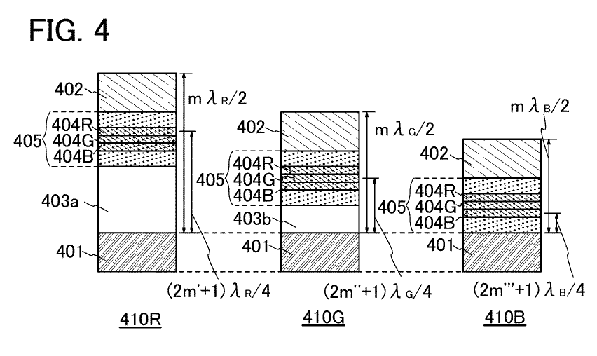

A light-emitting device described in this embodiment has a micro optical resonator (microcavity) structure in which a light resonant effect between a pair of electrodes is utilized. The light-emitting device includes a plurality of light-emitting elements each of which has at least an EL layer 405 between a pair of electrodes (a reflective electrode 401 and a semi-transmissive and semi-reflective electrode 402) as illustrated in FIG. 4. Further, the EL layer 405 includes at least a light-emitting layer 404 serving as a light-emitting region and may further include a hole-injection layer, a hole-transport layer, an electron-transport layer, an electron-injection layer, a charge-generation layer (E), and the like. Note that a heterocyclic compound of an embodiment of the present invention can be used for any of a hole-injection layer, a hole-transport layer, the light-emitting layer 404, and an electron-transport layer which are included in the EL layer 405.

In this embodiment, a light-emitting device will be described which includes light-emitting elements (a first light-emitting element (R) 410R, a second light-emitting element (G) 410G, and a third light-emitting element (B) 410B) having different structures as illustrated in FIG. 4.

The first light-emitting element (R) 410R has a structure in which a first transparent conductive layer 403a; an EL layer 405 including a first light-emitting layer (B) 404B, a second light-emitting layer (G) 404G and a third light-emitting layer (R) 404R in part; and a semi-transmissive and semi-reflective electrode 402 are sequentially stacked over a reflective electrode 401. The second light-emitting element (G) 410G has a structure in which a second transparent conductive layer 403b, the EL layer 405, and the semi-transmissive and semi-reflective electrode 402 are sequentially stacked over the reflective electrode 401. The third light-emitting element (B) 410B has a structure in which the EL layer 405 and the semi-transmissive and semi-reflective electrode 402 are sequentially stacked over the reflective electrode 401.

Note that the reflective electrode 401, the EL layer 405, and the semi-transmissive and semi-reflective electrode 402 are common to the light-emitting elements (the first light-emitting element (R) 410R, the second light-emitting element (G) 410G, and the third light-emitting element (B) 410B). The first light-emitting layer (B) 404B emits light (4) having a peak in a wavelength range from 420 nm to 480 nm. The second light-emitting layer (G) 404G emits light (λ_{G}) having a peak in a wavelength range from 500 nm to 550 nm. The third light-emitting layer (R) 404R emits light (λ_{R}) having a peak in a wavelength range from 600 nm to 760 nm. Thus, in each of the light-emitting elements (the first light-emitting element (R) 410R, the second light-emitting element (G) 410G, and the third light-emitting element (B) 410B), light emitted from the first light-emitting layer (B) 404B, light emitted from the second light-emitting layer (G) 404G and light emitted from the third light-emitting layer (R) 404R overlap with each other; accordingly, light having a broad emission spectrum that covers a visible light range can be emitted. Note that the above wavelengths satisfy the relation of λ_{B}<λ_{G}<λ_{R}.

Each of the light-emitting elements described in this embodiment has a structure in which the EL layer 405 is interposed between the reflective electrode 401 and the semi-transmissive and semi-reflective electrode 402. Light emission in all directions from the light-emitting layers included in the EL layer 405 is resonated by the reflective electrode 401 and the semi-transmissive and semi-reflective electrode 402 which function as a micro optical resonator (microcavity). Note that the reflective electrode 401 is formed using a conductive material having reflectivity, and a film whose visible light reflectivity is 40% to 100%, preferably 70% to 100%, and whose resistivity is 1×10⁻² Ωcm or lower is used. In addition, the semi-transmissive and semi-reflective electrode 402 is formed using a conductive material having reflectivity and a conductive material having a light-transmitting property, and a film whose visible light reflectivity is 20% to 80%, preferably 40% to 70%, and whose resistivity is 1×10⁻² Ωcm or lower is used.

In this embodiment, the thicknesses of the transparent conductive layers (the first transparent conductive layer 403a and the second transparent conductive layer 403b) provided in the first light-emitting element (R) 410R and the second light-emitting element (G) 410G, respectively, are varied between the light-emitting elements, whereby the light-emitting elements differ in the optical path length from the reflective electrode 401 to the semi-transmissive and semi-reflective electrode 402. In other words, in light having a broad emission spectrum, which is emitted from the light-emitting layers of each of the light-emitting elements, light with a wavelength that is resonated between the reflective electrode 401 and the semi-transmissive and semi-reflective electrode 402 can be enhanced while light with a wavelength that is not resonated therebetween can be attenuated. Thus, when the elements differ in the optical path length from the reflective electrode 401 to the semi-transmissive and semi-reflective electrode 402, light with different wavelengths can be extracted.

Note that the optical path length (also referred to as optical distance) is expressed as the product of an actual distance and a refractive index, and in this embodiment, is the product of an actual thickness and n (refractive index). That is, an optical path length=actual thickness×n.

Further, the total thickness from the reflective electrode 401 to the semi-transmissive and semi-reflective electrode 402 is set to mλ_{R}/2 (m is a natural number except 0) in the first light-emitting element (R) 410R; the total thickness from the reflective electrode 401 to the semi-transmissive and semi-reflective electrode 402 is set to mλ_{G}/2 (m is a natural number except 0) in the second light-emitting element (G) 410G; and the total thickness from the reflective electrode 401 to the semi-transmissive and semi-reflective electrode 402 is set to mλ_{B}/2 (m is a natural number except 0) in the third light-emitting element (B) 410B.

In this manner, the light (λ_{R}) emitted from the third light-emitting layer (R) 404R included in the EL layer 405 is mainly extracted from the first light-emitting element (R) 410R, the light (λ_{G}) emitted from the second light-emitting layer (G) 404G included in the EL layer 405 is mainly extracted from the second light-emitting element (G) 410G, and the light (λ_{B}) emitted from the first light-emitting layer (B) 404B included in the EL layer 405 is mainly extracted from the third light-emitting element (B) 410B. Note that the light extracted from each of the light-emitting elements is emitted from the semi-transmissive and semi-reflective electrode 402 side.

Further, strictly speaking, the total thickness from the reflective electrode 401 to the semi-transmissive and semi-reflective electrode 402 can be the total thickness from a reflection region in the reflective electrode 401 to a reflection region in the semi-transmissive and semi-reflective electrode 402. However, it is difficult to precisely determine the positions of the reflection regions in the reflective electrode 401 and the semi-transmissive and semi-reflective electrode 402. Hence, the above effect can be assumed to be sufficiently obtained wherever the reflection regions may be set in the reflective electrode 401 and the semi-transmissive and semi-reflective electrode 402.

Next, in the first light-emitting element (R) 410R, the optical path length from the reflective electrode 401 to the third light-emitting layer (R) 404R is adjusted to a desired thickness ((2m′+1)λ_{R}/4, where m′ is a natural number); thus, light emitted from the third light-emitting layer (R) 404R can be amplified. Light (first reflected light) that is reflected by the reflective electrode 401 of the light emission from the third light-emitting layer (R) 404R interferes with light (first incident light) that directly enters the semi-transmissive and semi-reflective electrode 402 from the third light-emitting layer (R) 404R. Therefore, by adjusting the optical path length from the reflective electrode 401 to the third light-emitting layer (R) 404R to the desired value ((2m′+1)λ_{R}/4, where m′ is a natural number), the phases of the first reflected light and the first incident light can be aligned with each other and the light emission from the third light-emitting layer (R) 404R can be amplified.

Note that, strictly speaking, the optical path length from the reflective electrode 401 to the third light-emitting layer (R) 404R can be the optical path length from a reflection region in the reflective electrode 401 to a light-emitting region in the third light-emitting layer (R) 404R. However, it is difficult to precisely determine the positions of the reflection region in the reflective electrode 401 and the light-emitting region in the third light-emitting layer (R) 404R. Hence, the above effect can be assumed to be sufficiently obtained wherever the reflection region and the light-emitting region may be set in the reflective electrode 401 and the third light-emitting layer (R) 404R, respectively.

Next, in the second light-emitting element (G) 410G, the optical path length from the reflective electrode 401 to the second light-emitting layer (G) 404G is adjusted to a desired thickness ((2m″+1)λ_{G}/4, where m″ is a natural number); thus, light emitted from the second light-emitting layer (G) 404G can be amplified. Light (second reflected light) that is reflected by the reflective electrode 401 of the light emission from the second light-emitting layer (G) 404G interferes with light (second incident light) that directly enters the semi-transmissive and semi-reflective electrode 402 from the second light-emitting layer (G) 404G. Therefore, by adjusting the optical path length from the reflective electrode 401 to the second light-emitting layer (G) 404G to the desired value ((2m″+1)λ_{G}/4, where m″ is a natural number), the phases of the second reflected light and the second incident light can be aligned with each other and the light emission from the second light-emitting layer (G) 404G can be amplified.

Note that, strictly speaking, the optical path length from the reflective electrode 401 to the second light-emitting layer (G) 404G can be the optical path length from a reflection region in the reflective electrode 401 to a light-emitting region in the second light-emitting layer (G) 404G. However, it is difficult to precisely determine the positions of the reflection region in the reflective electrode 401 and the light-emitting region in the second light-emitting layer (G) 404G. Hence, the above effect can be assumed to be sufficiently obtained wherever the reflection region and the light-emitting region may be set in the reflective electrode 401 and the second light-emitting layer (G) 404G, respectively.

Next, in the third light-emitting element (B) 410B, the optical path length from the reflective electrode 401 to the first light-emitting layer (B) 404B is adjusted to a desired thickness ((2m′″+1)λ_{B}/4, where m′″ is a natural number); thus, light emitted from the first light-emitting layer (B) 404B can be amplified. Light (third reflected light) that is reflected by the reflective electrode 401 of the light emission from the first light-emitting layer (B) 404B interferes with light (third incident light) that directly enters the semi-transmissive and semi-reflective electrode 402 from the first light-emitting layer (B) 404B. Therefore, by adjusting the optical path length from the reflective electrode 401 to the first light-emitting layer (B) 404B to the desired value ((2m′″+1)λ_{B}/4, where m′″ is a natural number), the phases of the third reflected light and the third incident light can be aligned with each other and the light emission from the first light-emitting layer (B) 404B can be amplified.

Note that, strictly speaking, the optical path length from the reflective electrode 401 to the first light-emitting layer (B) 404B in the third light-emitting element can be the optical path length from a reflection region in the reflective electrode 401 to a light-emitting region in the first light-emitting layer (B) 404B. However, it is difficult to precisely determine the positions of the reflection region in the reflective electrode 401 and the light-emitting region in the first light-emitting layer (B) 404B. Hence, the above effect can be assumed to be sufficiently obtained wherever the reflection region and the light-emitting region may be set in the reflective electrode 401 and the first light-emitting layer (B) 404B, respectively.

Note that although each of the light-emitting elements in the above-described structure includes a plurality of light-emitting layers in the EL layer, the present invention is not limited thereto; for example, the structure of the tandem light-emitting element which is described in Embodiment 4 can be combined, in which case a plurality of EL layers and a charge-generation layer interposed therebetween are provided in one light-emitting element and one or more light-emitting layers are formed in each of the EL layers.

The light-emitting device described in this embodiment has a microcavity structure, in which light with wavelengths which differ depending on the light-emitting elements can be extracted even when they include the same EL layers, so that it is not needed to form light-emitting elements for the colors of R, G and B. Therefore, the above structure is advantageous for full color display owing to easiness in achieving higher resolution display or the like. In addition, emission intensity with a predetermined wavelength in the front direction can be increased, whereby power consumption can be reduced. The above structure is particularly useful in the case of being applied to a color display (image display device) including pixels of three or more colors but may also be applied to lighting or the like.

### Embodiment 6

In this embodiment, a light-emitting device including a light-emitting element in which a heterocyclic compound of an embodiment of the present invention is used for a light-emitting layer will be described.

The light-emitting device can be either a passive matrix light-emitting device or an active matrix light-emitting device. Note that any of the light-emitting elements described in the other embodiments can be applied to the light-emitting device described in this embodiment.

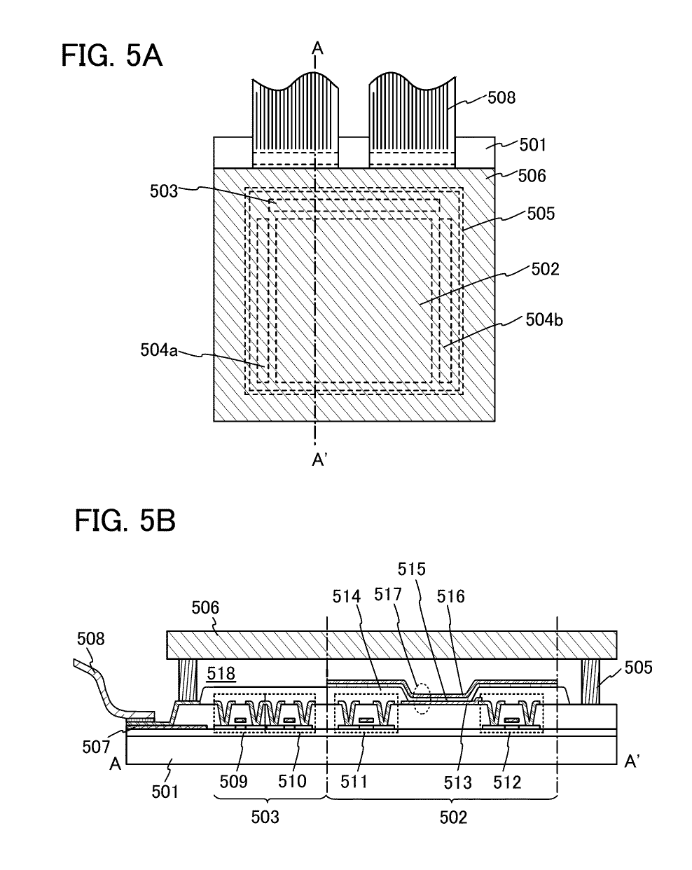

In this embodiment, an active matrix light-emitting device is described with reference to FIGS. 5A and 5B.

Note that FIG. 5A is a top view illustrating the light-emitting device and FIG. 5B is a cross-sectional view taken along chain line A-A′ in FIG. 5A. The active matrix light-emitting device according to this embodiment includes a pixel portion 502 provided over an element substrate 501, a driver circuit portion (a source line driver circuit) 503, and driver circuit portions (gate line driver circuits) 504a and 504b. The pixel portion 502, the driver circuit portion 503, and the driver circuit portions 504a and 504b are sealed between the element substrate 501 and the sealing substrate 506 by a sealant 505.

In addition, a lead wiring 507 is provided over the element substrate 501. The lead wiring 507 is provided for connecting an external input terminal through which a signal (e.g., a video signal, a clock signal, a start signal, and a reset signal) or a potential from the outside is transmitted to the driver circuit portion 503 and the driver circuit portions 504a and 504b. Here is shown an example in which a flexible printed circuit (FPC) 508 is provided as the external input terminal. Although only the FPC is illustrated here, a printed wiring board (PWB) may be attached to the FPC. The light-emitting device in this specification includes, in its category, not only the light-emitting device itself but also the light-emitting device provided with the FPC or the PWB.

Next, a cross-sectional structure is described with reference to FIG. 5B. The driver circuit portions and the pixel portion are formed over the element substrate 501; here are illustrated the driver circuit portion 503 which is the source line driver circuit and the pixel portion 502.

The driver circuit portion 503 is an example where a CMOS circuit is formed, which is a combination of an n-channel TFT 509 and a p-channel TFT 510. Note that a circuit included in the driver circuit portion may be formed using various CMOS circuits, PMOS circuits, or NMOS circuits. Although a driver integrated type in which the driver circuit is formed over the substrate is described in this embodiment, the driver circuit is not necessarily formed over the substrate, and the driver circuit can be formed outside, not over the substrate.

The pixel portion 502 is formed of a plurality of pixels each of which includes a switching TFT 511, a current control TFT 512, and a first electrode (anode) 513 which is electrically connected to a wiring (a source electrode or a drain electrode) of the current control TFT 512. Note that an insulator 514 is formed to cover end portions of the first electrode (anode) 513. In this embodiment, the insulator 514 is formed using a positive photosensitive acrylic resin.

The insulator 514 preferably has a curved surface with curvature at an upper end portion or a lower end portion thereof in order to obtain favorable coverage by a film which is to be stacked over the insulator 514. For example, in the case of using a positive photosensitive acrylic resin as a material for the insulator 514, the insulator 514 preferably has a curved surface with a curvature radius (0.2 μm to 3 μm) at the upper end portion. Note that the insulator 514 can be formed using either a negative photosensitive resin or a positive photosensitive resin. It is possible to use, without limitation to an organic compound, either an organic compound or an inorganic compound such as silicon oxide or silicon oxynitride.

An EL layer 515 and a second electrode (cathode) 516 are stacked over the first electrode (anode) 513. In the EL layer 515, at least a light-emitting layer is provided. Further, in the EL layer 515, a hole-injection layer, a hole-transport layer, an electron-transport layer, an electron-injection layer, a charge-generation layer, and the like can be provided as appropriate in addition to the light-emitting layer. Note that a heterocyclic compound of an embodiment of the present invention can be applied to the light-emitting layer, the hole-injection layer, the hole-transport layer, or the electron-transport layer.

A light-emitting element 517 is formed of a stacked structure of the first electrode (anode) 513, the EL layer 515, and the second electrode (cathode) 516. For the first electrode (anode) 513, the EL layer 515, and the second electrode (cathode) 516, the materials described in Embodiment 2 can be used. Although not illustrated, the second electrode (cathode) 516 is electrically connected to the FPC 508 which is the external input terminal.

Although the cross-sectional view of FIG. 5B illustrates only one light-emitting element 517, a plurality of light-emitting elements are arranged in matrix in the pixel portion 502. Light-emitting elements which provide three kinds of light emission (R, G, and B) are selectively formed in the pixel portion 502, whereby a light-emitting device capable of full color display can be formed. Alternatively, a light-emitting device which is capable of full color display may be manufactured by a combination with color filters.

Further, the sealing substrate 506 is attached to the element substrate 501 with the sealant 505, whereby the light-emitting element 517 is provided in a space 518 surrounded by the element substrate 501, the sealing substrate 506, and the sealant 505. The space 518 may be filled with an inert gas (such as nitrogen or argon), or the sealant 505.

An epoxy-based resin is preferably used for the sealant 505. It is preferable that such a material do not transmit moisture or oxygen as much as possible. As the sealing substrate 506, a glass substrate, a quartz substrate, or a plastic substrate formed of fiberglass reinforced plastic (FRP), polyvinyl fluoride (PVF), polyester, acrylic, or the like can be used.

As described above, an active matrix light-emitting device can be obtained.

Note that the structure described in this embodiment can be combined as appropriate with any of the structures described in the other embodiments.

### Embodiment 7

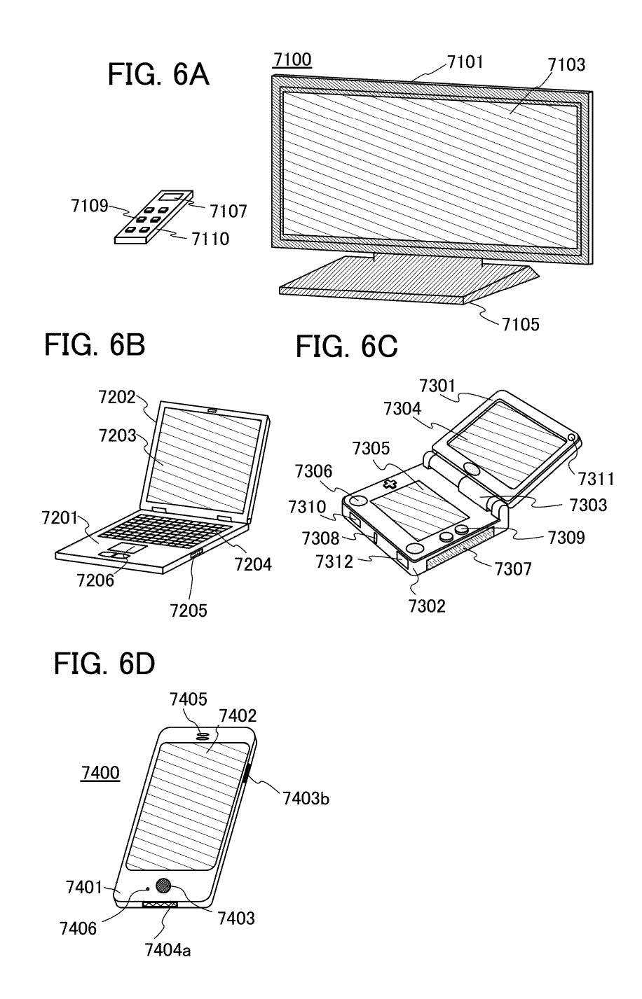

In this embodiment, examples of a variety of electronic devices which are completed using a light-emitting device are described with reference to FIGS. 6A to 6D.

The light-emitting device is fabricated using a light-emitting element including a heterocyclic compound of an embodiment of the present invention.

Examples of the electronic devices to which the light-emitting device is applied are a television device (also referred to as a television or a television receiver), a monitor of a computer or the like, a camera such as a digital camera or a digital video camera, a digital photo frame, a mobile phone (also referred to as cellular phone or cellular phone device), a portable game machine, a portable information terminal, an audio reproducing device, and a large-sized game machine such as a pachinko machine. Specific examples of these electronic devices are illustrated in FIGS. 6A to 6D.

FIG. 6A illustrates an example of a television set. In a television set 7100, a display portion 7103 is incorporated in a housing 7101. Images can be displayed on the display portion 7103, and the light-emitting device can be used for the display portion 7103. In addition, here, the housing 7101 is supported by a stand 7105.

Operation of the television set 7100 can be performed with an operation switch of the housing 7101 or a separate remote controller 7110. With operation keys 7109 of the remote controller 7110, channels and volume can be controlled and images displayed on the display portion 7103 can be controlled. Furthermore, the remote controller 7110 may be provided with a display portion 7107 for displaying data output from the remote controller 7110.

Note that the television set 7100 is provided with a receiver, a modem, and the like. With the receiver, a general television broadcast can be received. Furthermore, when the television set 7100 is connected to a communication network by wired or wireless connection via the modem, one-way (from a transmitter to a receiver) or two-way (between a transmitter and a receiver, between receivers, or the like) data communication can be performed.

FIG. 6B illustrates a computer having a main body 7201, a housing 7202, a display portion 7203, a keyboard 7204, an external connection port 7205, a pointing device 7206, and the like. Note that this computer is manufactured using the light-emitting device for the display portion 7203.

FIG. 6C illustrates a portable game machine having two housings, a housing 7301 and a housing 7302, which are connected with a joint portion 7303 so that the portable game machine can be opened or folded. A display portion 7304 is incorporated in the housing 7301, and a display portion 7305 is incorporated in the housing 7302. In addition, the portable game machine illustrated in FIG. 6C includes a speaker portion 7306, a recording medium insertion portion 7307, an LED lamp 7308, input means (an operation key 7309, a connection terminal 7310, a sensor 7311 (a sensor having a function of measuring force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared rays), and a microphone 7312), and the like. Needless to say, the structure of the portable game machine is not limited to the above as long as the light-emitting device is used for at least one of the display portion 7304 and the display portion 7305, and may include other accessories as appropriate. The portable game machine illustrated in FIG. 6C has a function of reading out a program or data stored in a storage medium to display it on the display portion, and a function of sharing information with another portable game machine by wireless communication. The portable game machine illustrated in FIG. 6C can have a variety of functions without limitation to the above.

FIG. 6D illustrates an example of a mobile phone. A mobile phone 7400 is provided with a display portion 7402 incorporated in a housing 7401, an operation button 7403, an external connection port 7404, a speaker 7405, a microphone 7406, and the like. Note that the mobile phone 7400 is manufactured using the light-emitting device for the display portion 7402.

When the display portion 7402 of the mobile phone 7400 illustrated in FIG. 6D is touched with a finger or the like, data can be input to the mobile phone 7400. Further, operations such as making a call and composing an e-mail can be performed by touching the display portion 7402 with a finger or the like.

There are mainly three screen modes of the display portion 7402. The first mode is a display mode mainly for displaying images. The second mode is an input mode mainly for inputting data such as text. The third mode is a display-and-input mode in which two modes of the display mode and the input mode are combined.

For example, in the case of making a call or composing an e-mail, a text input mode mainly for inputting text is selected for the display portion 7402 so that text displayed on the screen can be input. In this case, it is preferable to display a keyboard or number buttons on almost the entire screen of the display portion 7402.

When a detection device including a sensor for detecting inclination, such as a gyroscope or an acceleration sensor, is provided inside the mobile phone 7400, display on the screen of the display portion 7402 can be automatically switched by determining the orientation of the mobile phone 7400 (whether the mobile phone is placed horizontally or vertically for a landscape mode or a portrait mode).

The screen modes are switched by touching the display portion 7402 or operating the operation button 7403 of the housing 7401. The screen modes can also be switched depending on the kind of image displayed on the display portion 7402. For example, when a signal of an image displayed on the display portion is a signal of moving image data, the screen mode is switched to the display mode. When the signal is a signal of text data, the screen mode is switched to the input mode.

Moreover, in the input mode, when input by touching the display portion 7402 is not performed for a certain period while a signal detected by an optical sensor in the display portion 7402 is detected, the screen mode may be controlled so as to be switched from the input mode to the display mode.

The display portion 7402 may function as an image sensor. For example, an image of a palm print, a fingerprint, or the like is taken when the display portion 7402 is touched with the palm or the finger, whereby personal authentication can be performed. Further, by providing a backlight or a sensing light source which emits near-infrared light in the display portion, an image of a finger vein, a palm vein, or the like can be taken.

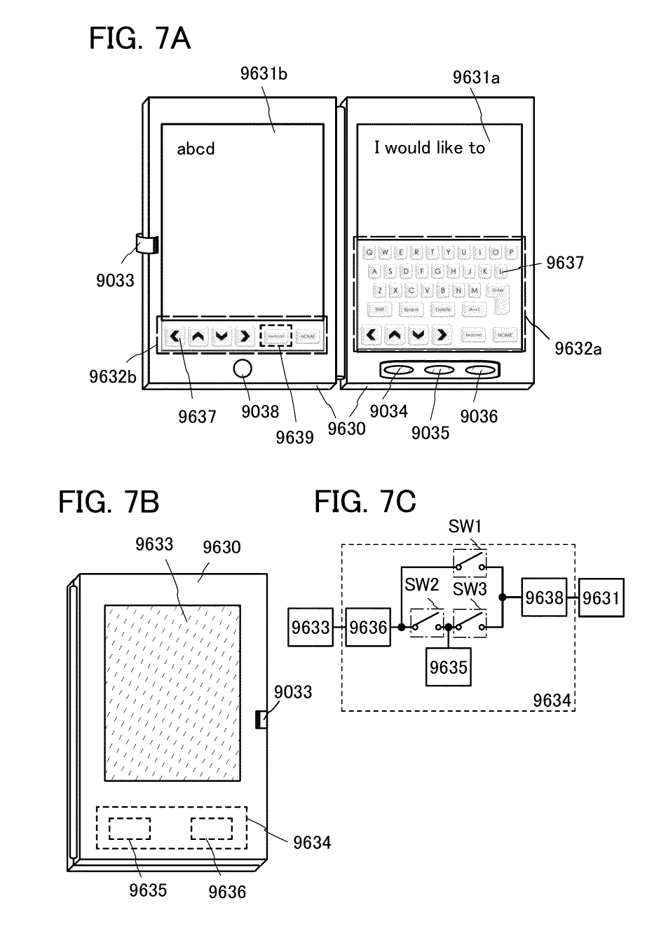

FIGS. 7A and 7B illustrate a tablet terminal that can be folded. In FIG. 7A, the tablet terminal is opened, and includes a housing 9630, a display portion 9631a, a display portion 9631b, a display-mode switching button 9034, a power button 9035, a power-saving-mode switching button 9036, a clip 9033, and an operation button 9038. The tablet terminal is manufactured using the light-emitting device for one or both of the display portion 9631a and the display portion 9631b.

A touch panel area 9632a can be provided in a part of the display portion 9631a, in which area, data can be input by touching displayed operation keys 9637. Note that half of the display portion 9631a has only a display function and the other half has a touch panel function. However, an embodiment of the present invention is not limited to this structure, and the whole display portion 9631a may have a touch panel function. For example, a keyboard can be displayed on the whole display portion 9631a to be used as a touch panel, and the display portion 9631b can be used as a display screen.

A touch panel area 9632b can be provided in part of the display portion 9631b like in the display portion 9631a. When a keyboard display switching button 9639 displayed on the touch panel is touched with a finger, a stylus, or the like, a keyboard can be displayed on the display portion 9631b.

The touch panel area 9632a and the touch panel area 9632b can be controlled by touch input at the same time.

The display-mode switching button 9034 allows switching between a landscape mode and a portrait mode, color display and black-and-white display, and the like. The power-saving-mode switching button 9036 allows optimizing the display luminance in accordance with the amount of external light in use which is detected by an optical sensor incorporated in the tablet terminal. In addition to the optical sensor, another detecting device such as a sensor for detecting inclination, like a gyroscope or an acceleration sensor, may be incorporated in the tablet terminal.

Although the display portion 9631a and the display portion 9631b have the same display area in FIG. 7A, an embodiment of the present invention is not limited to this example. The display portion 9631a and the display portion 9631b may have different areas or different display quality. For example, higher definition images may be displayed on one of the display portions 9631a and 9631b.

FIG. 7B illustrates the tablet terminal folded, which includes the housing 9630, a solar battery 9633, a charge and discharge control circuit 9634, a battery 9635, and a DCDC converter 9636. Note that FIG. 7B shows an example in which the charge and discharge control circuit 9634 includes the battery 9635 and the DCDC converter 9636.

Since the tablet terminal can be folded, the housing 9630 can be closed when not in use. Thus, the display portions 9631a and 9631b can be protected, which makes it possible to provide a tablet terminal with high durability and improved reliability for long-term use.

The tablet terminal illustrated in FIGS. 7A and 7B can have other functions such as a function of displaying various kinds of data (e.g., a still image, a moving image, and a text image), a function of displaying a calendar, a date, the time, or the like on the display portion, a touch-input function of operating or editing the data displayed on the display portion by touch input, and a function of controlling processing by various kinds of software (programs).

The solar battery 9633, which is attached on the surface of the tablet terminal, supplies electric power to a touch panel, a display portion, an image signal processor, and the like. Note that a structure in which the solar battery 9633 is provided is preferable because the battery 9635 which supplies electric power to the display portion 9631a and/or the display portion 9631b can be charged. The use of a lithium ion battery as the battery 9635 is advantageous in downsizing or the like.

The structure and operation of the charge and discharge control circuit 9634 illustrated in FIG. 7B are described with reference to a block diagram of FIG. 7C. FIG. 7C illustrates the solar battery 9633, the battery 9635, the DCDC converter 9636, a converter 9638, switches SW1 to SW3, and the display portion 9631. The battery 9635, the DCDC converter 9636, the converter 9638, and the switches SW1 to SW3 correspond to the charge and discharge control circuit 9634 in FIG. 7B.

First, description is made on an example of the operation in the case where power is generated by the solar battery 9633 using external light. The voltage of power generated by the solar battery is raised or lowered by the DCDC converter 9636 so that a voltage for charging the battery 9635 is obtained. When the display portion 9631 is operated with the power from the solar battery 9633, the switch SW1 is turned on and the voltage of the power is raised or lowered by the converter 9638 to a voltage needed for operating the display portion 9631. When display is not performed on the display portion 9631, the switch SW1 is turned off and the switch SW2 is turned on so that the battery 9635 can be charged.

Although the solar battery 9633 is shown as an example of a charge means, there is no particular limitation on the charge means and the battery 9635 may be charged with another means such as a piezoelectric element or a thermoelectric conversion element (Peltier element). For example, the battery 9635 may be charged with a non-contact power transmission module that transmits and receives power wirelessly (without contact) to charge the battery or with a combination of other charging means.

It is needless to say that an embodiment of the present invention is not limited to the electronic device illustrated in FIGS. 7A to 7C as long as the display portion described in the above embodiment is included.

As described above, the electronic devices can be obtained by the use of the light-emitting device according to an embodiment of the present invention. The light-emitting device has a remarkably wide application range, and can be applied to electronic devices in a variety of fields.

Note that the structure described in this embodiment can be combined as appropriate with any of the structures described in the other embodiments.

### Embodiment 8

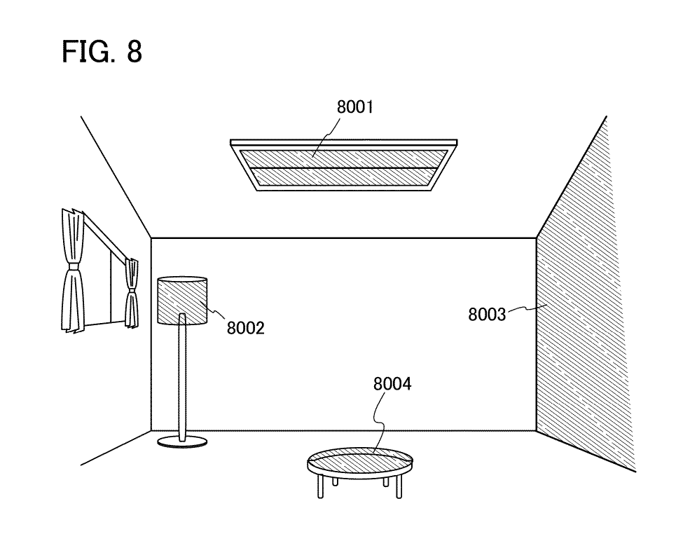

In this embodiment, examples of lighting devices which are completed using a light-emitting device will be described with reference to FIG. 8. The light-emitting device is fabricated using a light-emitting element including a heterocyclic compound of an embodiment of the present invention.

FIG. 8 illustrates an example in which the light-emitting device is used as an indoor lighting device 8001. Since the light-emitting device can have a larger area, it can be used for a lighting device having a large area. In addition, a lighting device 8002 in which a light-emitting region has a curved surface can also be obtained with the use of a housing with a curved surface. A light-emitting element included in the light-emitting device described in this embodiment is in a thin film form, which allows the housing to be designed more freely. Therefore, the lighting device can be elaborately designed in a variety of ways. Further, a wall of the room may be provided with a large-sized lighting device 8003.

Moreover, when the light-emitting device is used for a table by being used as a surface of a table, a lighting device 8004 which has a function as a table can be obtained. When the light-emitting device is used as part of other furniture, a lighting device which has a function as the furniture can be obtained.

In this manner, a variety of lighting devices to which the light-emitting device is applied can be obtained. Note that such lighting devices are also embodiments of the present invention.

Note that the structure described in this embodiment can be combined as appropriate with any of the structures described in the other embodiments.

### Example 1

### Synthesis Example 1

This example gives descriptions of a method of synthesizing 2-{3-[3-(2,8-diphenyldibenzothiophen-4-yl)phenyl]phenyl}dibenzo[f,h]quinoxaline (abbreviation: 2mDBTBPDBq-III), which is a heterocyclic compound of an embodiment of the present invention, represented by the structural formula (418) in Embodiment 1. A structure of 2mDBTBPDBq-III (abbreviation) is shown below.

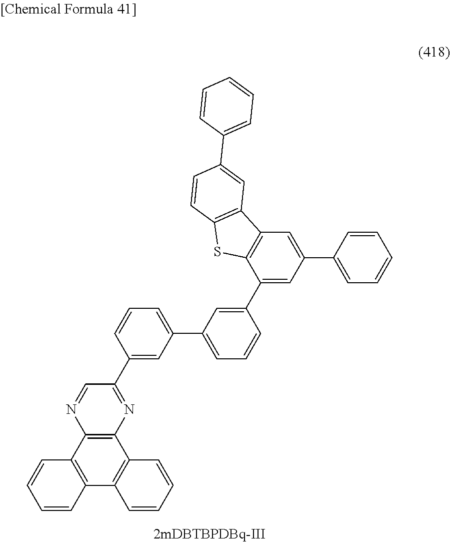 [SMILES: c1ccc(-c2ccc3sc4c(-c5cccc(-c6cccc(-c7cnc8c9ccccc9c9ccccc9c8n7)c6)c5)cc(-c5ccccc5)cc4c3c2)cc1]

### Step 1: Synthesis of 4-[3-(3-bromophenyl)phenyl]-2,8-diphenyldibenzothiophene

A synthesis scheme of 4-[3-(3-bromophenyl)phenyl]-2,8-diphenyldibenzothiophene is illustrated in (C-1).

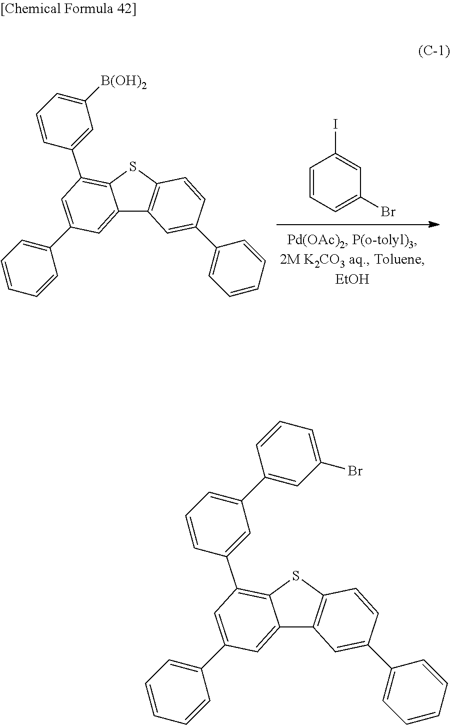 [SMILES: OB(O)c1cccc(-c2cc(-c3ccccc3)cc3c2sc2ccc(-c4ccccc4)cc23)c1 ; Brc1cccc(I)c1 ; Brc1cccc(-c2cccc(-c3cc(-c4ccccc4)cc4c3sc3ccc(-c5ccccc5)cc34)c2)c1]

In a 300 mL three-neck flask were put 6.5 g (23 mmol) of 3-bromoiodobenzene, 10 g (22 mmol) of 3-(2,8-diphenyldibenzothiophen-4-yl)phenylboronic acid, and 0.33 g (1.1 mmol) of tri(ortho-tolyl)phosphine. The air in the flask was replaced with nitrogen. To this mixture were added 80 mL of toluene, 30 mL of ethanol, and 25 mL of an aqueous solution of potassium carbonate (2.0 mol/L). This mixture was stirred to be degassed while the pressure was reduced. To this mixture was added 49 mg (0.22 mmol) of palladium(II)acetate, and the mixture was stirred at 80° C. for 3 hours under a nitrogen stream. Then, the aqueous layer of this mixture was extracted with toluene, and the extracted solution and the organic layer were combined and washed with saturated saline. The organic layer was dried with magnesium sulfate. After the drying, the mixture was subjected to gravity filtration. The obtained filtrate was concentrated to give a solid, and toluene/hexane was added to the solid. The mixture was irradiated with ultrasonic waves, whereby a solid was precipitated. This solid was collected by suction filtration to give 11 g of a target white powder in a yield of 96%.

### Step 2: Synthesis of 3-[3-(2,8-diphenyldibenzothiophen-4-yl)phenyl]phenylboronic acid

A synthesis scheme of 3-[3-(2,8-diphenyldibenzothiophen-4-yl)phenyl]phenylboronic acid is illustrated in (C-2).

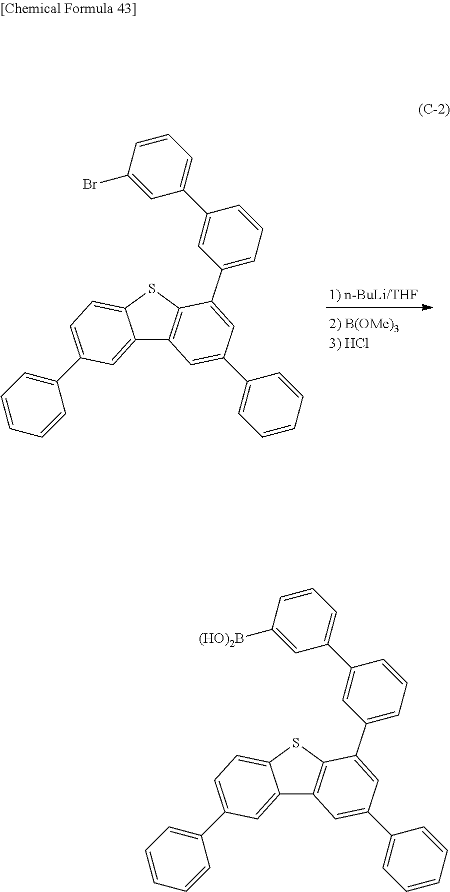 [SMILES: Brc1cccc(-c2cccc(-c3cc(-c4ccccc4)cc4c3sc3ccc(-c5ccccc5)cc34)c2)c1 ; OB(O)c1cccc(-c2cccc(-c3cc(-c4ccccc4)cc4c3sc3ccc(-c5ccccc5)cc34)c2)c1]

In a 500 mL three-neck flask was put 10 g (17 mmol) of 4-[3-(3-bromophenyl)phenyl]-2,8-diphenyldibenzothiophene. The air in the flask was replaced with nitrogen. To this mixture was added 176 mL of tetrahydrofuran (THF), and this solution was cooled to −80° C. Then, 11 mL (19 mmol) of n-butyllithium (a 1.6 mol/L hexane solution) was dripped into this solution with a syringe. After that, this solution was stirred at the same temperature for 2 hours. Then, 2.4 mL (21 mmol) of trimethyl borate was added to this solution, and the mixture was stirred for 16 hours while its temperature was returned to room temperature. After that, about 80 mL of dilute hydrochloric acid (1.0 mol/L) was added to this solution, followed by stirring for 2.5 hours. Then, the aqueous layer of this mixture was extracted with ethyl acetate, and the extracted solution and the organic layer were combined and washed with a saturated aqueous solution of sodium hydrogen carbonate and saturated saline. The organic layer was dried with magnesium sulfate. After the drying, the mixture was subjected to gravity filtration. The obtained filtrate was concentrated, so that a solid substance was obtained. The solid was recrystallized with ethyl acetate/hexane, so that 3.5 g of a target pale brown powder was obtained in a yield of 37%.

### Step 3: Synthesis of 2mDBTBPDBq-III (abbreviation)

A synthesis scheme of 2mDBTBPDBq-III (abbreviation) is illustrated in (C-3).

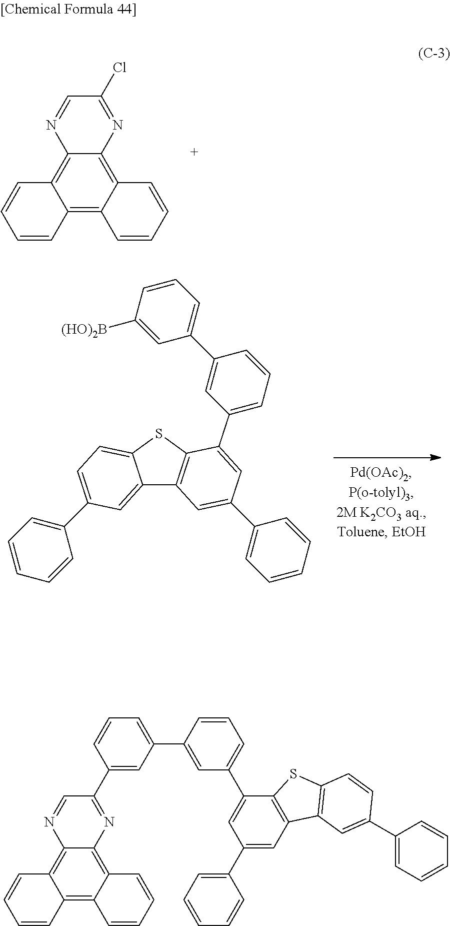

In a 300 mL three-neck flask were put 1.7 g (6.5 mmol) of 2-chlorodibenzo[f,h]quinoxaline and 3.4 g (6.5 mmol) of 3-[3-(2,8-diphenyldibenzothiophen-4-yl)phenyl]phenylboronic acid. The air in the flask was replaced with nitrogen. To this mixture were added 43 mL of toluene, 54 mL of ethanol, and 6.5 mL of an aqueous solution of sodium carbonate (2.0 mol/L). This mixture was stirred to be degassed while the pressure was reduced. To this mixture was added 75 mg (0.065 mmol) of tetrakis(triphenylphosphine)palladium(0), and the mixture was stirred at 80° C. for 4 hours under a nitrogen stream, so that a solid was precipitated. Then, 200 mL of water was added to this mixture and the mixture was stirred for 30 minutes. After the stirring, suction filtration was performed on the mixture, and a solid was obtained. To the obtained mixture was added 200 mL of ethanol. Then, irradiation of ultrasonic waves was performed and the solid was washed. After the washing, the mixture was subjected to suction filtration to give a solid. The solid was dried under reduced pressure. After the drying, the solid was dissolved in 800 mL of hot toluene, and this solution was subjected to suction filtration through Celite and alumina A solid obtained by concentration of the resulting filtrate was purified by silica gel column chromatography (the developing solvent was a mixed solvent of a 2:1 ratio of hexane to toluene) to give a solid. The obtained solid was purified by HPLC to give a solid. The obtained solid was dried under reduced pressure, so that 0.72 g of a target white solid was obtained in a yield of 15%.

By a train sublimation method, 0.72 g of the obtained white solid was purified. In the purification, 2mDBTBPDBq-III (abbreviation) was heated at 360° C. under a pressure of 2.8 Pa with a flow rate of an argon gas at 5.0 mL/min. Through the purification, 0.61 g of a white solid of 2mDBTBPDBq-III (abbreviation) was obtained at a collection rate of 84%.

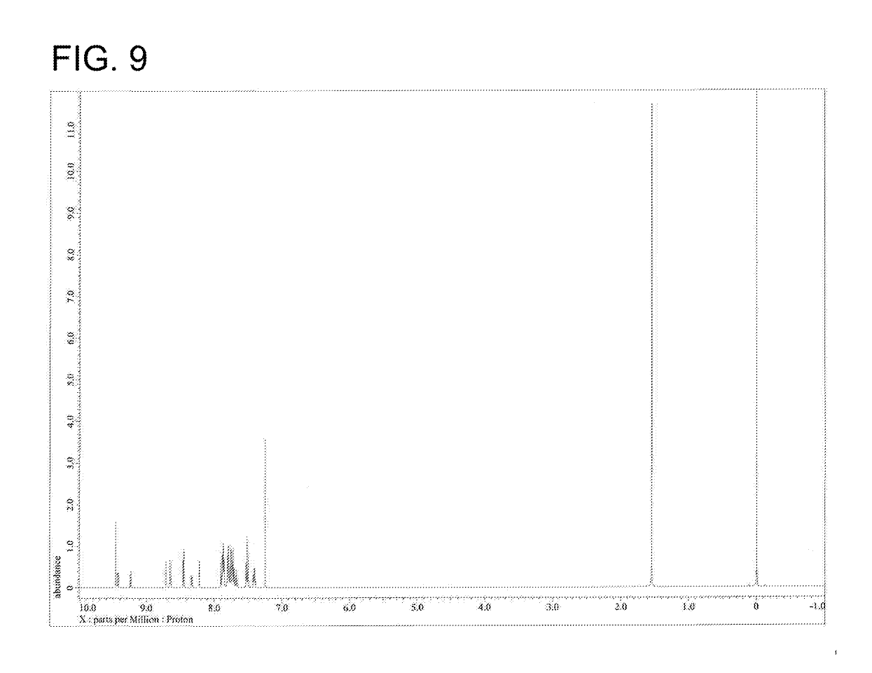

An analysis result of nuclear magnetic resonance spectroscopy (¹H-NMR) of the compound obtained by the above synthesis method is shown below. The ¹H-NMR chart is shown in FIG. 9. The result reveals that 2mDBTBPDBq-III (abbreviation), which is the heterocyclic compound of an embodiment of the present invention represented by the structural formula (418), was obtained.

¹H NMR (CDCl₃, 500 MHz): δ=7.39-7.43 (m, 2H), 7.52 (ddd, J=8.0, 1.7 Hz, 4H), 7.66-7.90 (m, 16H), 8.23 (t, J=1.7 Hz, 1H), 8.34 (d, J=2.4 Hz, 1H), 8.46 (dd, J=6.3, 1.7 Hz, 2H), 8.65 (d, J=8.0 Hz, 2H), 8.71 (t, J=1.7 Hz, 1H), 9.25 (dd, J=5.0, 1.1 Hz, 1H), 9.44 (dd, J=3.0, 1.1 Hz, 1H), 9.47 (s, 1H).

Next, 2mDBTBPDBq-III (abbreviation) obtained in this example was analyzed by liquid chromatography mass spectrometry (LC/MS).

The LC/MS was carried out with Acquity UPLC (produced by Waters Corporation) and Xevo G2 T of MS (produced by Waters Corporation).

In the MS, ionization was carried out by an electrospray ionization (ESI) method. At this time, the capillary voltage and the sample cone voltage were set to 3.0 kV and 30 V, respectively, and detection was performed in a positive mode.

A component which underwent the ionization under the above-described conditions was collided with an argon gas in a collision cell to dissociate into a plurality of product ions. Energy (collision energy) for the collision with argon was 70 eV. The mass range for the measurement was m/z=100 to 1200.

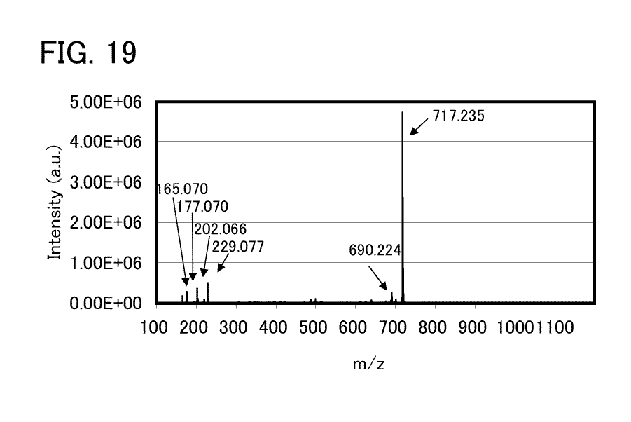

FIG. 19 shows the measurement result. The result in FIG. 19 shows that product ions of 2mDBTBPDBq-III (abbreviation), which is the heterocyclic compound of an embodiment of the present invention represented by the structural formula (418), are detected mainly around m/z=690, m/z=229, m/z=202, m/z=177, and m/z=165.

Note that the result in FIG. 19 shows characteristics derived from 2mDBTBPDBq-III (abbreviation) and therefore can be regarded as important data for identifying 2mDBTBPDBq-III (abbreviation) contained in the mixture.

The product ion around m/z=690 is assumed to be a cation in a state where one C atom and one N atom are detached from the dibenzo[f,h]quinoxaline ring of the compound represented by the structural formula (418), which is one feature of the heterocyclic compound of an embodiment of the present invention. Further, the product ion around m/z=229 is assumed to be a cation of a diazatriphenylenyl group such as dibenzo[f,h]quinoxaline. Moreover, the product ions around m/z=202, m/z=177, and m/z=165 are also detected at the same time. Therefore, it is indicated that 2mDBTBPDBq-III (abbreviation) which is the heterocyclic compound of an embodiment of the present invention includes a dibenzo[f,h]quinoxaline ring.

### Example 2

### Synthesis Example 2

This example gives descriptions of a method for synthesizing 2-{3-[2,8-bis(biphenyl-3-yl)dibenzothiophen-4-yl]phenyl}dibenzo[f,h]quinoxaline (abbreviation: 2mDBTPDBq-VI), which is a heterocyclic compound of an embodiment of the present invention represented by the structural formula (400) in Embodiment 1. A structure of 2mDBTPDBq-VI (abbreviation) is shown below.

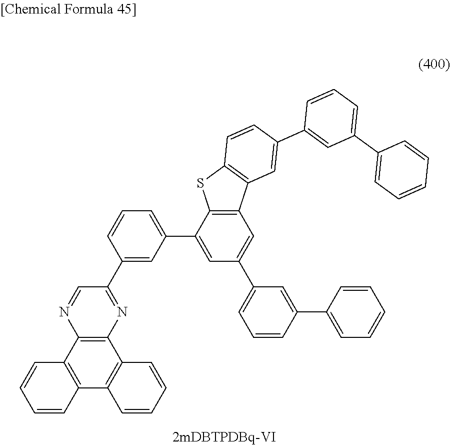 [SMILES: c1ccc(-c2cccc(-c3ccc4sc5c(-c6cccc(-c7cnc8c9ccccc9c9ccccc9c8n7)c6)cc(-c6cccc(-c7ccccc7)c6)cc5c4c3)c2)cc1]

### Step 1: Synthesis of 2,8-bis(biphenyl-3-yl)dibenzothiophene

A synthesis scheme of 2,8-bis(biphenyl-3-yl)dibenzothiophene is illustrated in (D-1).

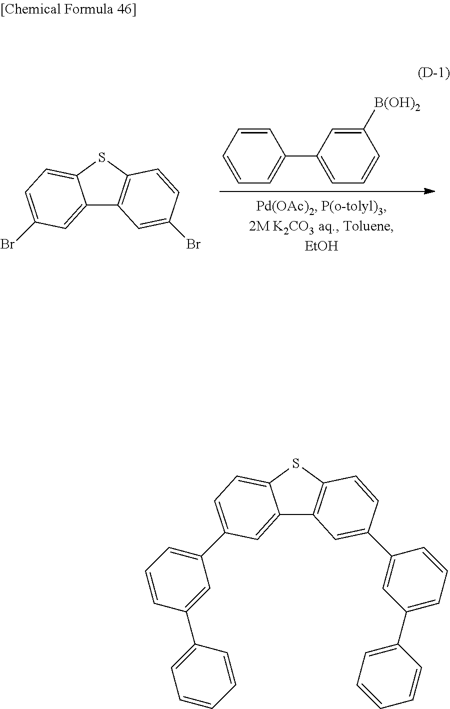 [SMILES: Brc1ccc2sc3ccc(Br)cc3c2c1 ; OB(O)c1cccc(-c2ccccc2)c1 ; c1ccc(-c2cccc(-c3ccc4sc5ccc(-c6cccc(-c7ccccc7)c6)cc5c4c3)c2)cc1]

In a 3.0 L three-neck flask were put 50 g (0.14 mol) of 2,8-dibromodibenzothiophene, 69 g (0.35 mmol) of 3-biphenylboronic acid, 4.4 g (14 mmol) of tri(ortho-tolyl)phosphine, 60 g (0.43 mol) of potassium carbonate, 380 mL of water, 1.2 L of toluene, and 120 mL of ethanol. This mixture was stirred to be degassed while the pressure was reduced. To this mixture was added 0.65 g (2.9 mmol) of palladium(II)acetate, and the mixture was stirred at 80° C. for 3 hours under a nitrogen stream. After the stirring, the aqueous layer of this mixture was extracted with toluene, and the extracted solution and the organic layer were combined and washed with saturated saline. The organic layer was dried with magnesium sulfate, and this mixture was subjected to gravity filtration. A solid obtained by concentration of the resulting filtrate was dissolved in about 500 mL of toluene. This solution was suction filtered through Celite, alumina, and Florisil. Toluene/hexane was added to a solid obtained by concentration of the obtained filtrate and the mixture was irradiated with ultrasonic waves and washed. The solid was collected by suction filtration, so that 60 g of a target white powder was obtained in a yield of 84%.

### Step 2: Synthesis of 2,8-bis(biphenyl-3-yl)dibenzothiophen-4-ylboronic acid

A synthesis scheme of 2,8-bis(biphenyl-3-yl)dibenzothiophen-4-ylboronic acid is illustrated in (D-2).

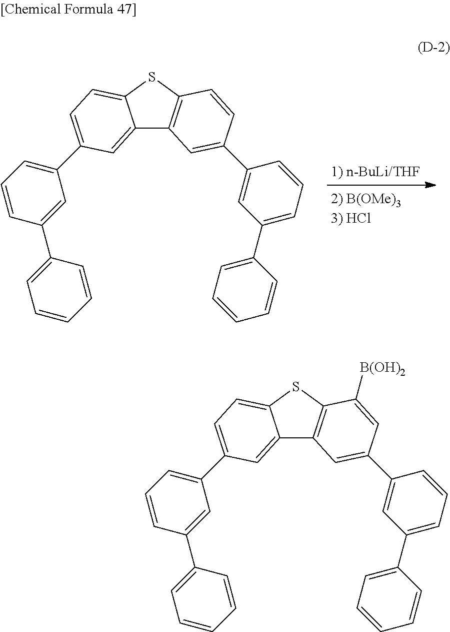 [SMILES: c1ccc(-c2cccc(-c3ccc4sc5ccc(-c6cccc(-c7ccccc7)c6)cc5c4c3)c2)cc1 ; OB(O)c1cc(-c2cccc(-c3ccccc3)c2)cc2c1sc1ccc(-c3cccc(-c4ccccc4)c3)cc12]

In a 2.0 L three-neck flask was put 36 g (73 mmol) of 2,8-bis(biphenyl-3-yl)dibenzothiophene. The air in the flask was replaced with nitrogen. In the flask was put 370 mL of tetrahydrofuran (THF), and this solution was cooled to −80° C. Then, 50 mL (80 mmol) of n-butyllithium (a 1.6 mol/L hexane solution) was dripped to this solution with a dripping funnel. After the dripping, this solution was stirred for 2 hours while its temperature was returned to room temperature. After the stirring, this solution was again cooled to −80° C., and 11 mL (100 mmol) of trimethyl borate was added to this solution, followed by stirring for 18 hours while its temperature was returned to room temperature. After the stirring, about 200 mL of dilute hydrochloric acid (1.0 mol/L) was added to this solution and the solution was stirred for 1 hour. After the stirring, the aqueous layer of this mixture was extracted with ethyl acetate, and the extracted solution and the organic layer were combined and washed with a saturated aqueous solution of sodium hydrogen carbonate and saturated saline. The organic layer was dried with magnesium sulfate, and then the mixture was subjected to gravity filtration. The obtained filtrate was concentrated to give a solid. Ethyl acetate/toluene was added to the obtained solid, the mixture was irradiated with ultrasonic waves, and a solid was collected by suction filtration, so that 33 g of a target white powder was obtained in a yield of 87%.

### Step 3: Synthesis of 4-(3-bromophenyl)-2,8-bis(biphenyl-3-yl)dibenzothiophene

A synthesis scheme of 4-(3-bromophenyl)-2,8-bis(biphenyl-3-yl)dibenzothiophene is illustrated in (D-3).

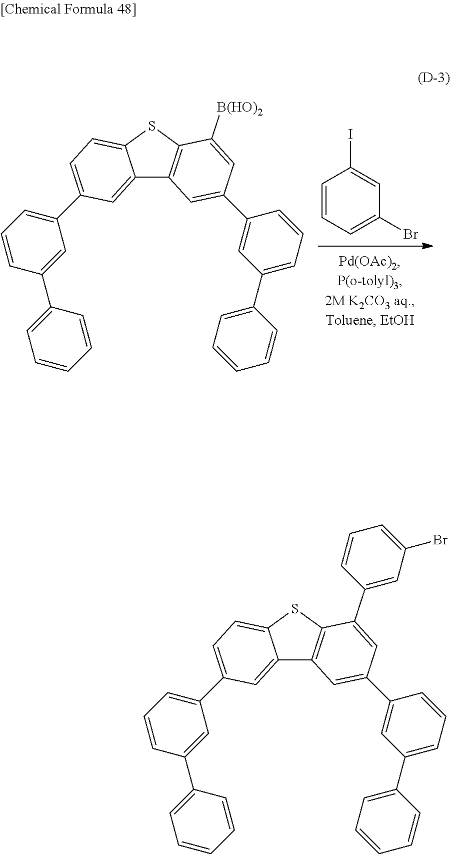

In a 1.0 L three-neck flask were put 4.8 mL (37 mmol) of 3-bromoiodobenzene, 20 g (37 mmol) of 2,8-bis(biphenyl-3-yl)dibenzothiophen-4-ylboronic acid, and 1.5 g (5.6 mmol) of tri(ortho-tolyl)phosphine. The air in the flask was replaced with nitrogen. To this mixture were added 140 mL of toluene, 47 mL of ethanol, 10 g of potassium carbonate, and 37 mL of water. This mixture was stirred to be degassed while the pressure was reduced. To this mixture was added 0.16 g (1.1 mmol) of palladium(II)acetate and the mixture was stirred at 80° C. for 6 hours under a nitrogen stream. After that, the aqueous layer of this mixture was extracted with toluene, and the extracted solution and the organic layer were combined and washed with saturated saline. The organic layer was dried with magnesium sulfate and this mixture was subjected to gravity filtration. Toluene/methanol was added to an oily substance obtained by concentration of the obtained filtrate, and the mixture was irradiated with ultrasonic waves to precipitate a solid. The precipitated solid was collected by suction filtration, whereby 22 g of a target pale brown solid was obtained in a yield of 94%.

### Step 4: Synthesis of 3-[2,8-bis(biphenyl-3-yl)dibenzothiophen-4-yl]phenylboronic acid

A synthesis scheme of 3-[2,8-bis(biphenyl-3-yl)dibenzothiophen-4-yl]phenylboronic acid is illustrated in (D-4).

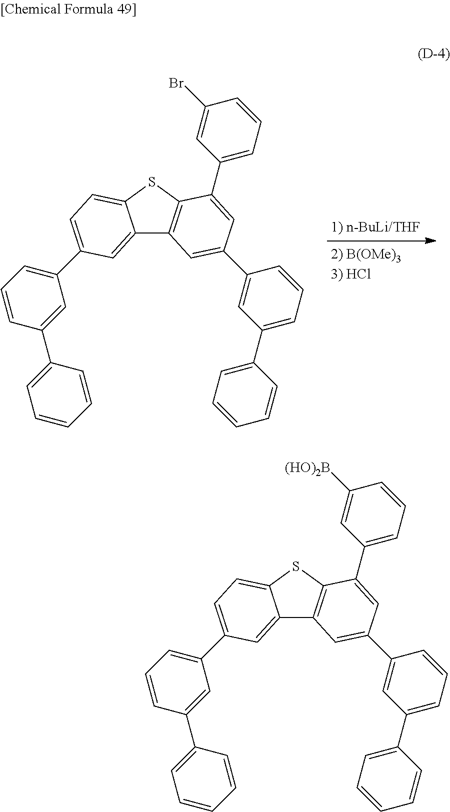 [SMILES: Brc1cccc(-c2cc(-c3cccc(-c4ccccc4)c3)cc3c2sc2ccc(-c4cccc(-c5ccccc5)c4)cc23)c1 ; OB(O)c1cccc(-c2cc(-c3cccc(-c4ccccc4)c3)cc3c2sc2ccc(-c4cccc(-c5ccccc5)c4)cc23)c1]

In a 500 mL three-neck flask was put 20 g (31 mmol) of 4-(3-bromophenyl)-2,8-bis(biphenyl-3-yl)dibenzothiophene. The air in the flask was replaced with nitrogen. In the flask was put 310 mL of tetrahydrofuran (THF), and the solution was cooled to −80° C. To this solution, 21 mL (34 mmol) of n-butyllithium (a 1.6 mol/L hexane solution) was dripped with a syringe. After the dripping, this solution was stirred at the same temperature for 2 hours. After the stirring, 4.2mL (37 mmol) of trimethyl borate was added to this solution, and the solution was stirred for 18 hours while its temperature was returned to room temperature. After the stirring, about 10 mL of dilute hydrochloric acid (1.0 mol/L) was added to the solution, and the mixture was stirred for 1 hour. Then, the aqueous layer of this mixture was extracted with ethyl acetate, and the extracted solution and the organic layer were combined and washed with a saturated aqueous solution of sodium hydrogen carbonate and saturated saline. The organic layer was dried with magnesium sulfate. After the drying, the mixture was subjected to gravity filtration. The obtained filtrate was concentrated to give a solid. The obtained solid was dissolved in heated ethyl acetate and hexane was added for recrystallization, so that 12 g of a target pale brown powder was obtained in a yield of 63%.

### Step 5: Synthesis of 2mDBTPDBq-VI (abbreviation)

A synthesis scheme of 2mDBTPDBq-VI (abbreviation) is illustrated in (D-5).

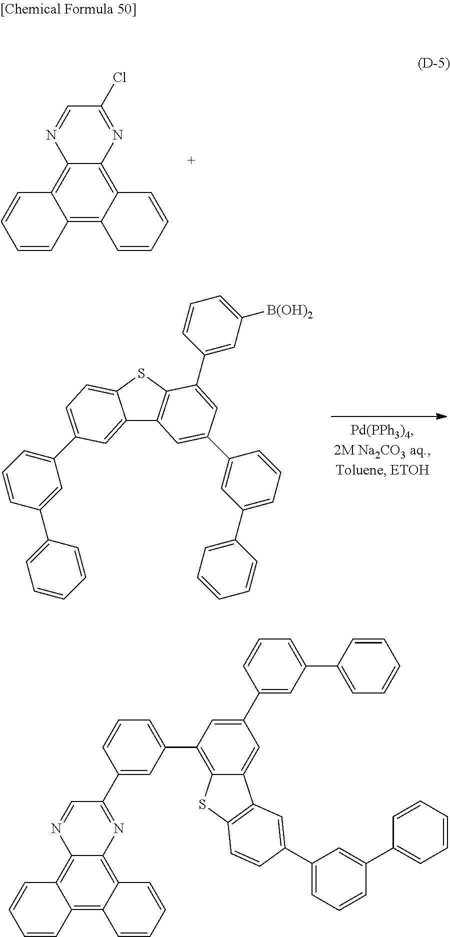 [SMILES: Clc1cnc2c3ccccc3c3ccccc3c2n1 ; OB(O)c1cccc(-c2cc(-c3cccc(-c4ccccc4)c3)cc3c2sc2ccc(-c4cccc(-c5ccccc5)c4)cc23)c1 ; c1ccc(-c2cccc(-c3ccc4sc5c(-c6cccc(-c7cnc8c9ccccc9c9ccccc9c8n7)c6)cc(-c6cccc(-c7ccccc7)c6)cc5c4c3)c2)cc1]

In a 300 mL three-neck flask were put 5.1 g (19 mmol) of 2-chlorodibenzo[f,h]quinoxaline, 12 g (2.4 mmol) of 3-[2,8-bis(biphenyl-3-yl)dibenzothiophen-4-yl]phenylboronic acid, 130 mL of toluene, 13 mL of ethanol, and 20 mL of an aqueous solution of sodium carbonate (2.0 mol/L). This mixture was stirred to be degassed while the pressure was reduced. To this mixture was added 0.23 g (0.20 mmol) of tetrakis(triphenylphosphine)palladium(0), and the mixture was stirred at 80° C. for 3.5 hours under a nitrogen stream, so that a solid was precipitated. After the stirring, 200 mL of water was added to this mixture and the mixture was stirred at room temperature for 30 minutes. Then, the mixture was subjected to suction filtration to give a solid. To the obtained solid was added 200 mL of ethanol and the solid was irradiated with ultrasonic waves and washed. After the washing, this mixture was subjected to suction filtration to give a solid. The obtained solid was dried under reduced pressure. After the drying, the solid was dissolved in 800 mL of hot toluene, and the solution was subjected to suction filtration through Celite and alumina. A solid obtained by concentration of the obtained filtrate was dried under reduced pressure, so that 7.2 g of a target white powder was obtained in a yield of 46%.

By a train sublimation method, 7.2 g of the obtained white powdery solid was purified. In the purification, 2mDBTPDBq-VI (abbreviation) was heated at 395° C. under a pressure of 3.5 Pa with a flow rate of an argon gas at 15 mL/min. Through the purification, 6.2 g of a white solid of 2mDBTPDBq-VI (abbreviation) was obtained at a collection rate of 86%.

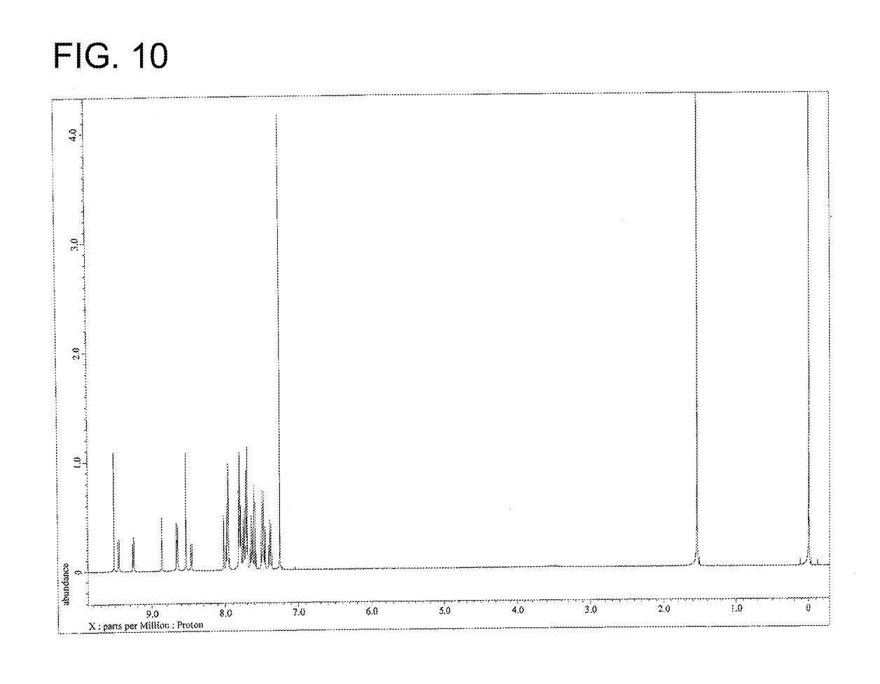

A result of nuclear magnetic resonance spectroscopy (¹H-NMR) of the compound obtained by the above synthesis method is shown below. The ¹H-NMR chart is shown in FIG. 10. The result reveals that 2mDBTPDBq-VI (abbreviation), which is the heterocyclic compound of an embodiment of the present invention represented by the structural formula (400), was obtained.

¹H NMR (CDCl₃, 500 MHz): δ=7.38 (ddd, J=8.6, 7.5 Hz, 2H), 7.48 (ddd, J=8.1, 7.4 Hz, 4H), 7.59-7.66 (m, 4H), 7.70-7.82 (m, 12H), 7.95-7.99 (m, 4H), 8.02 (dd, J=1.7 Hz, 1H), 8.46 (d, J=8.0 Hz, 1H), 8.53 (d, J=1.8 Hz, 2H), 8.65 (d, J=8.0 Hz, 2H), 8.87 (dd, J=1.7 Hz, 1H), 9.25 (dd, J=6.3, 1.7 Hz, 1H), 9.45 (dd, J=6.8, 1.2 Hz, 1H), 9.51 (s, 1H).

Next, 2mDBTPDBq-VI (abbreviation) obtained in this example was analyzed by liquid chromatography mass spectrometry (LC/MS).

The LC/MS was carried out with Acquity UPLC (produced by Waters Corporation) and Xevo G2 T of MS (produced by Waters Corporation).

In the MS, ionization was carried out by an electrospray ionization (ESI) method. At this time, the capillary voltage and the sample cone voltage were set to 3.0 kV and 30 V, respectively, and detection was performed in a positive mode.

A component which underwent the ionization under the above-described conditions was collided with an argon gas in a collision cell to dissociate into a plurality of product ions. Energy (collision energy) for the collision with argon was 70 eV. The mass range for the measurement was m/z=100 to 1200.

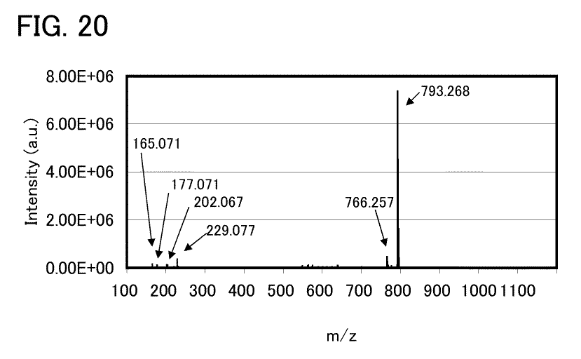

FIG. 20 shows the measurement result. The result in FIG. 20 shows that product ions of 2mDBTPDBq-VI (abbreviation), which is the heterocyclic compound of an embodiment of the present invention represented by the structural formula (400), are detected mainly around m/z=766, m/z=229, and m/z=165.

Note that the result in FIG. 20 shows characteristics derived from 2mDBTPDBq-VI (abbreviation) and therefore can be regarded as important data for identifying 2mDBTPDBq-VI (abbreviation) contained in the mixture.

The product ion around m/z=766 is assumed to be a cation in a state where one C atom and one N atom are detached from the dibenzo[f,h]quinoxaline ring of the compound represented by the structural formula (400), which is one feature of the heterocyclic compound of an embodiment of the present invention. Further, the product ion around m/z=229 is assumed to be a cation of a diazatriphenylenyl group such as dibenzo[f,h]quinoxaline. Moreover, the product ions around m/z=202, m/z=177, and m/z=165 are also detected at the same time. Therefore, it is indicated that 2mDBTPDBq-VI (abbreviation) which is the heterocyclic compound of an embodiment of the present invention includes a dibenzo[f,h]quinoxaline ring.

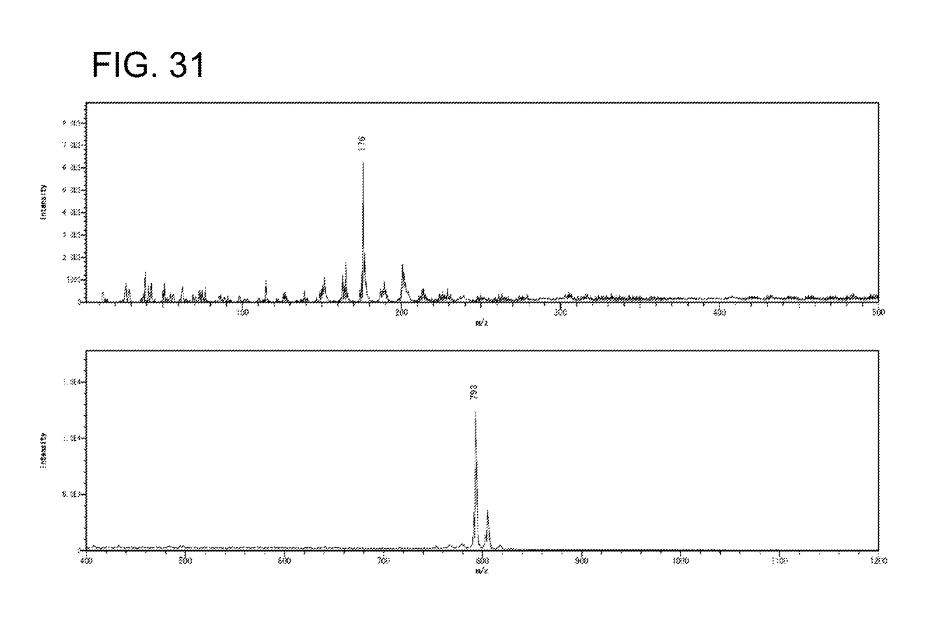

Further, 2mDBTPDBq-VI (abbreviation), which is an embodiment of the present invention, was measured with a time-of-flight secondary ion mass spectrometer (TOF-SIMS); FIG. 31 shows the obtained qualitative spectrum in the case of a positive ion.

TOF.SIMS 5 (produced by ION-TOF GmbH) was used, and Bi₃⁺⁺ was used as a primary ion source. Note that irradiation with the primary ions was performed in a pulsed manner with a pulse width of 7 nm to 12 nm. The irradiation amount was greater than or equal to 8.2×10¹⁰ ions/cm² and less than or equal to 6.7×10¹¹ ions/cm² (less than or equal to 1×10¹² ions/cm²), the acceleration voltage was 25 keV, and the current value was 0.2 pA. A powder of 2mDBTPDBq-VI (abbreviation) was the sample used for the measurement.

The result of analysis by TOF-SIMS (positive ion) in FIG. 31 shows that product ions of 2mDBTPDBq-VI (abbreviation) (m/z=792.26), which is the heterocyclic compound of an embodiment of the present invention represented by the structural formula (400), are detected mainly around m/z=176. Here, the expression “around” indicates that difference in values of product ions, which change depending on whether a hydrogen ion or an isotope is present or not, is allowable. Since the product ions shown in the result in FIG. 31 are similar to the product ions of 2mDBTPDBq-VI (abbreviation) in FIG. 20, which were detected by the MS analysis (positive ion), the result of the measurement by TOF-SIMS can also be regarded as important data for identifying 2mDBTPDBq-VI (abbreviation) contained in the mixture.

### Example 3

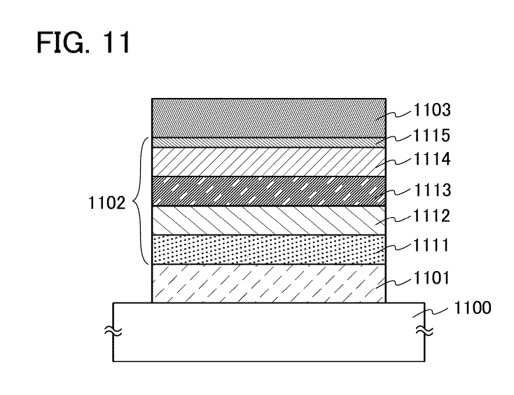

This example gives descriptions of a light-emitting element 1 in which 2-{3-[2,8-bis(biphenyl-3-yl)dibenzothiophen-4-yl]phenyl}dibenzo[t h]quinoxaline (abbreviation: 2mDBTPDBq-VI) (structural formula (400)) which is the heterocyclic compound of an embodiment of the present invention is used for part of a light-emitting layer, with reference to FIG. 11. Note that chemical formulae of materials used in this example are illustrated below.

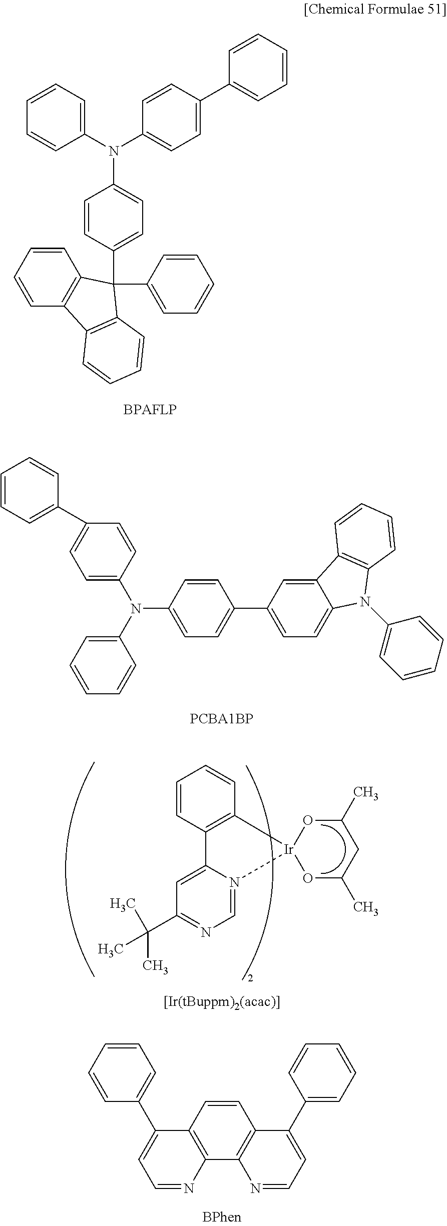

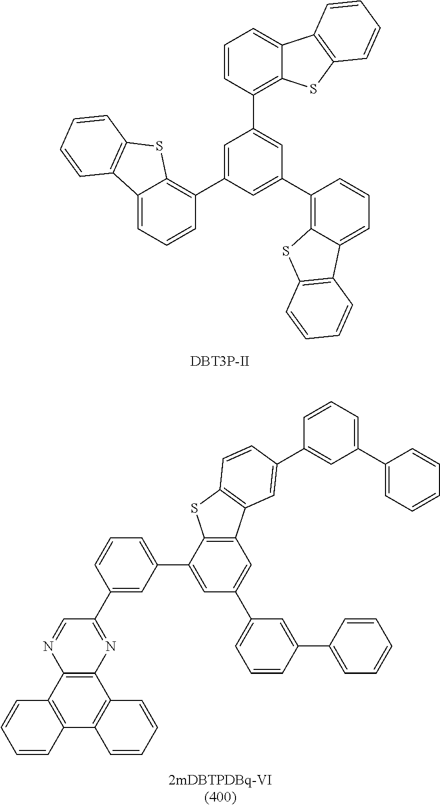 [SMILES: c1ccc2c(c1)sc1c(-c3cc(-c4cccc5c4sc4ccccc45)cc(-c4cccc5c4sc4ccccc45)c3)cccc12 ; "DBT3P-II" ; c1ccc(-c2cccc(-c3ccc4sc5c(-c6cccc(-c7cnc8c9ccccc9c9ccccc9c8n7)c6)cc(-c6cccc(-c7ccccc7)c6)cc5c4c3)c2)cc1]

### <Fabrication of Light-Emitting Element 1>

First, indium tin oxide containing silicon oxide (ITSO) was deposited over a glass substrate 1100 by a sputtering method, so that a first electrode 1101 which functions as an anode was formed. The thickness was 110 nm and the electrode area was 2 mm×2 mm.

Then, as pretreatment for forming the light-emitting element over the substrate 1100, UV ozone treatment was performed for 370 seconds after washing of a surface of the substrate with water and baking that was performed at 200° C. for 1 hour.

After that, the substrate was transferred into a vacuum evaporation apparatus where the pressure had been reduced to approximately 10⁻⁴ Pa, and was subjected to vacuum baking at 170° C. for 30 minutes in a heating chamber of the vacuum evaporation apparatus, and then the substrate 1100 was cooled down for about 30 minutes.

Next, the substrate 1100 was fixed to a holder provided in the vacuum evaporation apparatus so that the surface of the substrate 1100 over which the first electrode 1101 was formed faced downward. In this example, a case is described in which a hole-injection layer 1111, a hole-transport layer 1112, a light-emitting layer 1113, an electron-transport layer 1114, and an electron-injection layer 1115 which are included in an EL layer 1102 are sequentially formed by a vacuum evaporation method.

After reducing the pressure of the vacuum evaporation apparatus to 10⁻⁴ Pa, 1,3,5-tri(dibenzothiophen-4-yl)benzene (abbreviation: DBT3P-II) and molybdenum(VI) oxide were co-evaporated with a mass ratio of DBT3P-II (abbreviation) to molybdenum oxide being 4:2, whereby the hole-injection layer 1111 was formed over the first electrode 1101. The thickness of the hole-injection layer 1111 was 40 nm. Note that the co-evaporation is an evaporation method in which some different substances are evaporated from some different evaporation sources at the same time.

Then, 4-phenyl-4′-(9-phenylfluoren-9-yl)triphenylamine (abbreviation: BPAFLP) was evaporated to a thickness of 20 nm, so that the hole-transport layer 1112 was formed.

Next, the light-emitting layer 1113 was formed over the hole-transport layer 1112. Co-evaporated were 2-{3-[2,8-bis(biphenyl-3-yl)dibenzothiophen-4-yl]phenyl}dibenzo[f,h]quinoxaline (abbreviation: 2mDBTPDBq-VI), 4-phenyl-4′-(9-phenyl-9H-carbazol-3-yl)triphenylamine (abbreviation: PCBA1BP), and (acetylacetonato)bis(6-tert-butyl-4-phenylpyrimidinato)iridium(III) (abbreviation: [Ir(tBuppm)₂(acac)]) with a mass ratio of 2mDBTPDBq-VI (abbreviation) to PCBA1BP (abbreviation) and [Ir(tBuppm)₂(acac)] (abbreviation) being 0.8:0.2:0.05. The thickness of the light-emitting layer 1113 was 40 nm. Thus, the light-emitting layer 1113 was formed.

Then, 2mDBTPDBq-VI (abbreviation) was evaporated to a thickness of 10 nm over the light-emitting layer 1113 and bathophenanthroline (abbreviation: Bphen) was evaporated to a thickness of 20 nm, whereby the electron-transport layer 1114 having a stacked structure was formed. Furthermore, lithium fluoride was evaporated to a thickness of 1 nm over the electron-transport layer 1114, whereby the electron-injection layer 1115 was formed.

Finally, aluminum was evaporated to a thickness of 200 nm over the electron-injection layer 1115 to form a second electrode 1103 serving as a cathode; thus, the light-emitting element 1 was obtained. Note that in all the above evaporation steps, evaporation was performed by a resistance-heating method.

An element structure of the light-emitting element 1 obtained as described above is shown in Table 1.

TABLE 1

| | | Hole- | Hole- | Light- | Electron- | | Electron- | |
|---|---|---|---|---|---|---|---|---|
| | First | injection | transport | emitting | transport | | injection | Second |
| | Electrode | Layer | Layer | Layer | Layer | | Layer | Electrode |
| Light- | ITSO | DBT3P-II: | BPAFLP | *1 | **1 | Bphen | LiF | Al |
| emitting | (110 nm) | MoOx | (20 nm) | | | (20 nm) | (1 nm) | (200 nm) |
| Element 1 | | (4:2, 40 nm) | | | | | | |

*1 2mDBTPDBq-VI:PCBA1BP:[Ir(tBuppm)₂(acac)] (0.8:0.2:0.05, 40 nm)
**1 2mDBTPDBq-VI (10 nm)

Further, the manufactured light-emitting element 1 was sealed in a glove box containing a nitrogen atmosphere so as not to be exposed to the air (a sealant was applied onto an outer edge of the element and heat treatment was performed at 80° C. for 1 hour at the time of sealing).

### <Operation Characteristics of Light-Emitting Element 1>

Operation characteristics of the manufactured light-emitting element 1 were measured. Note that the measurement was carried out at room temperature (under an atmosphere in which the temperature was kept at 25° C.).

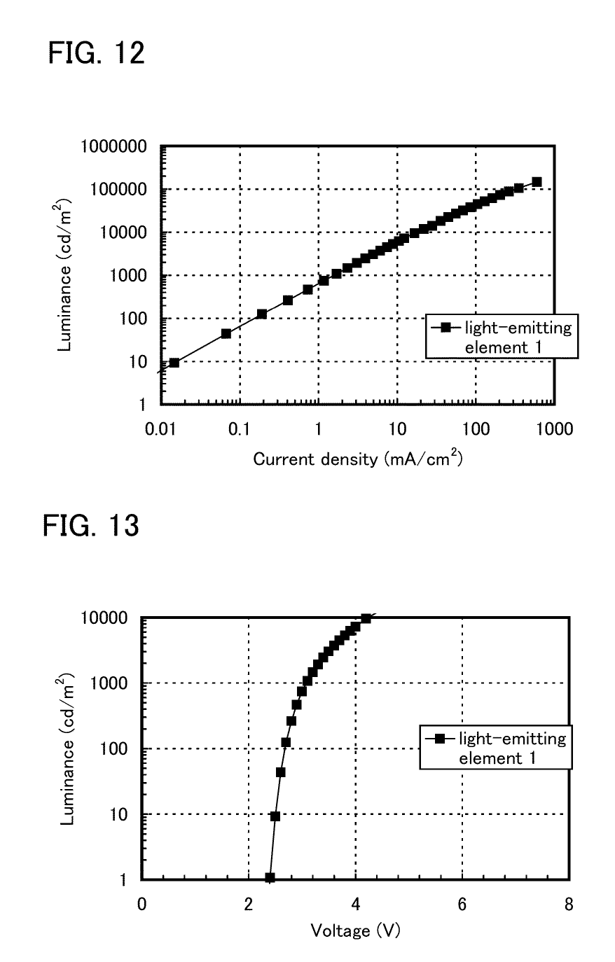

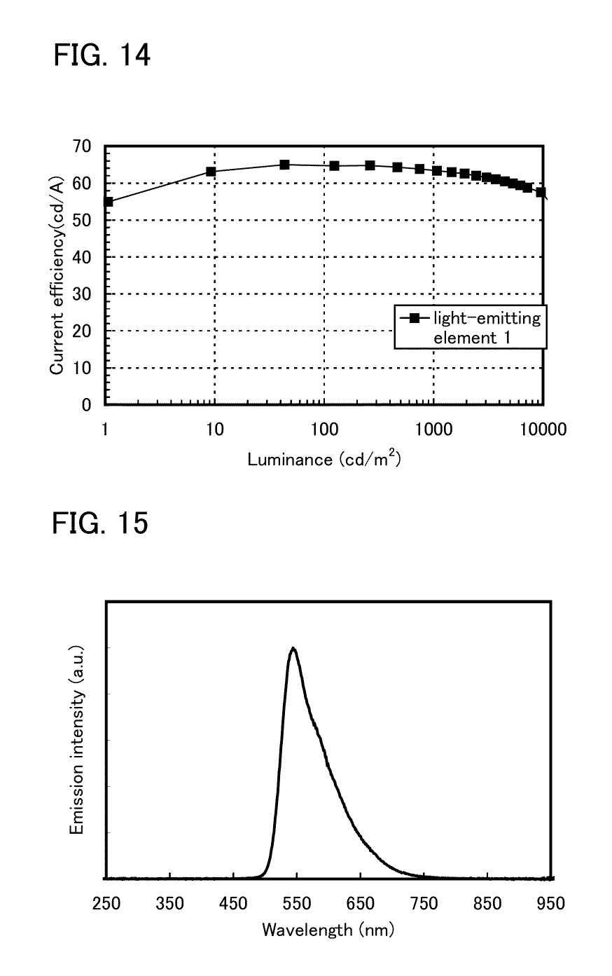

FIG. 12 shows luminance versus current density characteristics, FIG. 13 shows luminance versus voltage characteristics, and FIG. 14 shows current efficiency versus luminance characteristics of the light-emitting element 1.

FIG. 14 suggests that the light-emitting element 1 in which the heterocyclic compound of an embodiment of the present invention is used for part of the light-emitting layer as a host material has low power consumption and high efficiency.

Table 2 below shows initial values of main characteristics of the light-emitting element 1 at a luminance of about 1000 cd/m².

TABLE 2

| | | | Current | | | Current | Power | External |
|---|---|---|---|---|---|---|---|---|
| | Voltage | Current | Density | Chromaticity | Luminance | Efficiency | Efficiency | Quantum |
| | (V) | (mA) | (mA/cm²) | (x, y) | (cd/m²) | (cd/A) | (lm/W) | Efficiency (%) |
| Light- | 3.1 | 0.068 | 1.7 | (0.42, 0.56) | 1100 | 63 | 64 | 18 |
| emitting | | | | | | | | |
| Element 1 | | | | | | | | |

The above results in Table 2 also suggest that the light-emitting element 1 manufactured in this example has high luminance and high current efficiency.

FIG. 15 shows an emission spectrum when a current at a current density of 0.1 mA/cm² was supplied to the light-emitting element 1. FIG. 15 shows that the emission spectrum of the light-emitting element 1 has a peak at around 544 nm, which indicates that the emission spectrum is derived from emission of [Ir(tBuppm)₂(acac)] (abbreviation) included in the light-emitting layer 1113.

Thus, it was found that 2mDBTPDBq-VI (abbreviation) has a high T1 level and can be used for a host material or a carrier-transport material in a light-emitting element which exhibits phosphorescence in a visible light region (wavelength longer than or equal to that of blue light).

### Example 4

This example gives descriptions of a light-emitting element 2in which 2-[3′-(dibenzothiophen-4-yl)biphenyl-3-yl]dibenzo[f,h]quinoxaline (abbreviation: 2mDBTBPDBq-II) (structural formula (103)) which is the heterocyclic compound of an embodiment of the present invention is used for part of a light-emitting layer, a light-emitting element 3in which 2-[3-(2,8-diphenyldibenzothiophen-4-yl)phenyl]dibenzo[f,h]quinoxaline (abbreviation: 2mDBTPDBq-III) (structural formula (113)) which is the heterocyclic compound of an embodiment of the present invention is used for part of a light-emitting layer, and a comparative light-emitting element in which 2-[3-(dibenzothiophen-4-yl)phenyl]dibenzo[f,h]quinoxaline (abbreviation: 2mDBTPDBq-II) (structural formula (600)) is used for part of a light-emitting layer. Note that FIG. 11, which is used for explanation of the light-emitting element 1 in Example 3, is used for explanation of the light-emitting element 2, the light-emitting element 3, and the comparative light-emitting element in this example. Chemical formulae of materials used in this example are illustrated below.

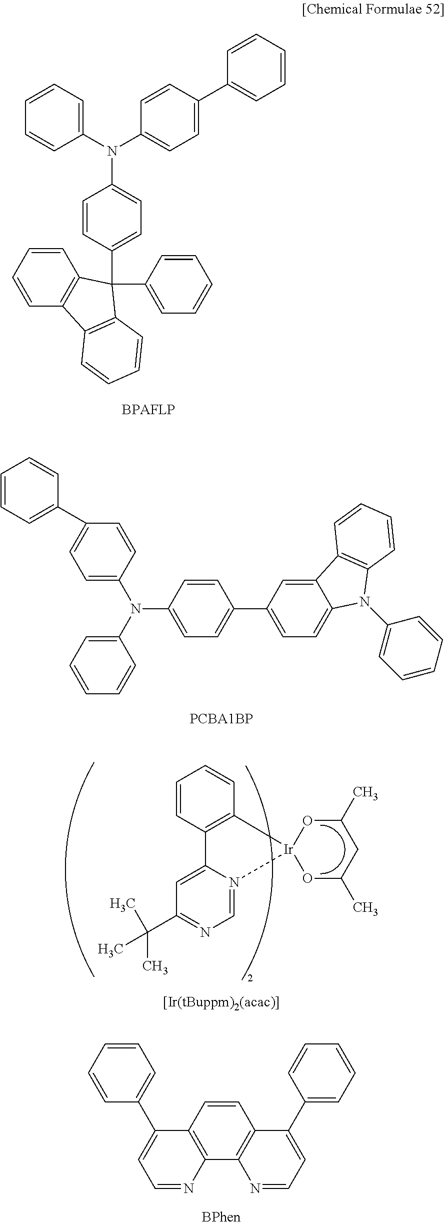

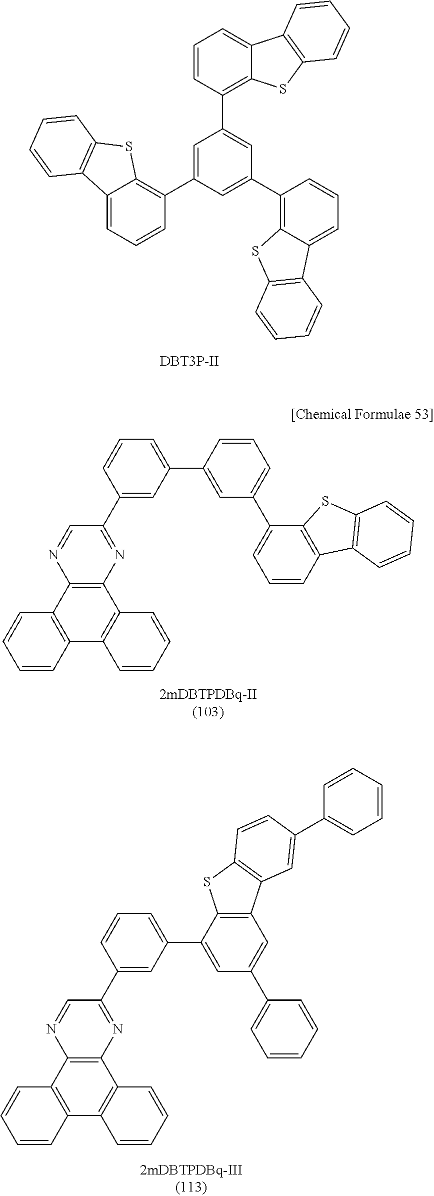 [SMILES: c1ccc2c(c1)sc1c(-c3cc(-c4cccc5c4sc4ccccc45)cc(-c4cccc5c4sc4ccccc45)c3)cccc12 ; "DBT3P-II" ; c1cc(-c2cccc(-c3cccc4c3sc3ccccc34)c2)cc(-c2cnc3c4ccccc4c4ccccc4c3n2)c1 ; c1ccc(-c2ccc3sc4c(-c5cccc(-c6cnc7c8ccccc8c8ccccc8c7n6)c5)cc(-c5ccccc5)cc4c3c2)cc1]

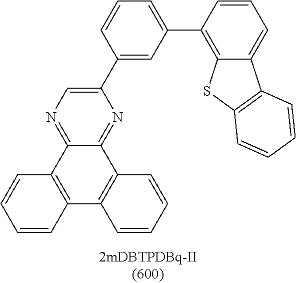 [SMILES: c1cc(-c2cnc3c4ccccc4c4ccccc4c3n2)cc(-c2cccc3c2sc2ccccc23)c1]

### <Fabrication of Light-Emitting Element 2, Light-Emitting Element 3, and Comparative Light-Emitting Element>

First, indium tin oxide containing silicon oxide (ITSO) was deposited over a glass substrate 1100 by a sputtering method, so that a first electrode 1101 which functions as an anode was formed. The thickness was 110 nm and the electrode area was 2 mm×2 mm.

Then, as pretreatment for forming the light-emitting element over the substrate 1100, UV ozone treatment was performed for 370 seconds after washing of a surface of the substrate with water and baking that was performed at 200° C. for 1 hour.

After that, the substrate was transferred into a vacuum evaporation apparatus where the pressure had been reduced to approximately 10⁻⁴ Pa, and was subjected to vacuum baking at 170° C. for 30 minutes in a heating chamber of the vacuum evaporation apparatus, and then the substrate 1100 was cooled down for about 30 minutes.

Next, the substrate 1100 was fixed to a holder provided in the vacuum evaporation apparatus so that the surface of the substrate 1100 over which the first electrode 1101 was formed faced downward. In this example, a case is described in which a hole-injection layer 1111, a hole-transport layer 1112, a light-emitting layer 1113, an electron-transport layer 1114, and an electron-injection layer 1115 which are included in an EL layer 1102 are sequentially formed by a vacuum evaporation method.

After reducing the pressure of the vacuum evaporation apparatus to 10⁻⁴ Pa, 1,3,5-tri(dibenzothiophen-4-yl)benzene (abbreviation: DBT3P-II) and molybdenum(VI) oxide were co-evaporated with a mass ratio of DBT3P-II (abbreviation) to molybdenum oxide being 1:0.5, whereby the hole-injection layer 1111 was formed over the first electrode 1101. The thickness of the hole-injection layer 1111 was 40 nm. Note that the co-evaporation is an evaporation method in which some different substances are evaporated from some different evaporation sources at the same time.

Then, 4-phenyl-4′-(9-phenylfluoren-9-yl)triphenylamine (abbreviation: BPAFLP) was evaporated to a thickness of 20 nm, so that the hole-transport layer 1112 was formed. Note that the steps up to here are common to the light-emitting element 2, the light-emitting element 3, and the comparative light-emitting element.

Next, the light-emitting layer 1113 was formed over the hole-transport layer 1112.

In the light-emitting element 2, co-evaporated were 2-[3′-(dibenzothiophen-4-yl)biphenyl-3-yl]dibenzo[f,h]quinoxaline (abbreviation: 2mDBTBPDBq-II), 4-phenyl-4′-(9-phenyl-9H-carbazol-3-yl)triphenylamine (abbreviation: PCBA1BP), and (acetylacetonato)bis(6-tert-butyl-4-phenylpyrimidinato)iridium(III) (abbreviation: [Ir(tBuppm)₂(acac)]) with a mass ratio of 2mDBTBPDBq-II (abbreviation) to PCBA1BP (abbreviation) and [Ir(tBuppm)₂(acac)]being 0.8:0.2:0.05. The thickness of the light-emitting layer 1113 was 40 nm. Thus, the light-emitting layer 1113 was formed.

In the light-emitting element 3, co-evaporated were 2-[3-(2,8-diphenyldibenzothiophen-4-yl)phenyl]dibenzo[f,h]quinoxaline (abbreviation: 2mDBTPDBq-III), PCBA1BP (abbreviation), and [Ir(tBuppm)₂(acac)] (abbreviation) with a mass ratio of 2mDBTPDBq-III (abbreviation) to PCBA1BP (abbreviation) and [Ir(tBuppm)₂(acac)] being 0.8:0.2:0.05. The thickness of the light-emitting layer 1113 was 40 nm. Thus, the light-emitting layer 1113 was formed.

In the comparative light-emitting element, co-evaporated were 2-[3-(dibenzothiophen-4-yl)phenyl]dibenzo[f,h]quinoxaline (abbreviation: 2mDBTPDBq-II), PCBA1BP (abbreviation), and [Ir(tBuppm)₂(acac)] (abbreviation) with a mass ratio of 2mDBTPDBq-II (abbreviation) to PCBA1BP (abbreviation) and [Ir(tBuppm)₂(acac)] being 0.8:0.2:0.05. The thickness of the light-emitting layer 1113 was 40 nm. Thus, the light-emitting layer 1113 was formed.

Then, in each of the light-emitting element 2, the light-emitting element 3, and the comparative light-emitting element, 2mDBTPDBq-II (abbreviation) was evaporated to a thickness of 10 nm over the light-emitting layer 1113 and bathophenanthroline (abbreviation: Bphen) was evaporated to a thickness of 20 nm, whereby the electron-transport layer 1114 having a stacked structure was formed. Furthermore, lithium fluoride was evaporated to a thickness of 1 nm over the electron-transport layer 1114, whereby the electron-injection layer 1115 was formed.

Finally, aluminum was evaporated to a thickness of 200 nm over the electron-injection layer 1115 to form a second electrode 1103 serving as a cathode; thus, the light-emitting element 2, the light-emitting element 3, and the comparative light-emitting element were obtained. Note that in all the above evaporation steps, evaporation was performed by a resistance-heating method.

Note that in each of the light-emitting element 2, the light-emitting element 3, and the comparative light-emitting element, the steps after formation of the light-emitting layer 1113 were performed in a similar manner.

Table 3 shows element structures of the light-emitting element 2, the light-emitting element 3, and the comparative light-emitting element obtained as described above.

TABLE 3

| | | | Hole- | Light- | Electron- | | Electron- | |
|---|---|---|---|---|---|---|---|---|
| | First | Hole-injection | transport | emitting | transport | | injection | Second |
| | Electrode | Layer | Layer | Layer | Layer | | Layer | Electrode |
| Light-emitting | ITSO | DBT3P-II:MoOx | BPAFLP | *2 | **2 | Bphen | LiF | Al |
| Element 2 | (110 nm) | (1:0.5, 40 nm) | (20 nm) | | | (20 nm) | (1 nm) | (200 nm) |
| Light-emitting | ITSO | DBT3P-II:MoOx | BPAFLP | *3 | **3 | Bphen | LiF | Al |
| Element 3 | (110 nm) | (1:0.5, 40 nm) | (20 nm) | | | (20 nm) | (1 nm) | (200 nm) |
| Comparative | ITSO | DBT3P-II:MoOx | BPAFLP | *0 | **0 | Bphen | LiF | Al |
| Light-emitting | (110 nm) | (1:0.5, 40 nm) | (20 nm) | | | (20 nm) | (1 nm) | (200 nm) |
| Element | | | | | | | | |

*2 2mDBTBPDBq-II:PCBA1BP:[Ir(tBuppm)₂(acac)] (0.8:0.2:0.05, 40 nm)
**2 2mDBTBPDBq-II (10 nm)
*3 2mDBTPDBq-III:PCBA1BP:[Ir(tBuppm)₂(acac)] (0.8:0.2:0.05, 40 nm)
**3 2mDBTPDBq-III (10 nm)
*0 2mDBTPDBq-II:PCBA1BP:[Ir(tBuppm)₂(acac)] (0.8:0.2:0.05, 40 nm)
**0 2mDBTPDBq-II (10 nm)

Further, the manufactured light-emitting element 2, the light-emitting element 3, and the comparative light-emitting element were each sealed in a glove box containing a nitrogen atmosphere so as not to be exposed to the air (a sealant was applied onto an outer edge of each element and heat treatment was performed at 80° C. for 1 hour at the time of sealing).

### <Heat-Resisting Properties of Light-Emitting Element 2, Light-Emitting Element 3, and Comparative Light-Emitting Element>

Heat-resistance test was conducted on the manufactured light-emitting element 2, light-emitting element 3, and comparative light-emitting element. The evaluation was performed in such a manner that each light-emitting element was preserved in a constant temperature bath kept at 80° C. for a predetermined period and then current efficiency was measured. Note that the current efficiency was measured at room temperature (in an atmosphere kept at 25° C.) after the light-emitting elements were taken out of the constant temperature bath.

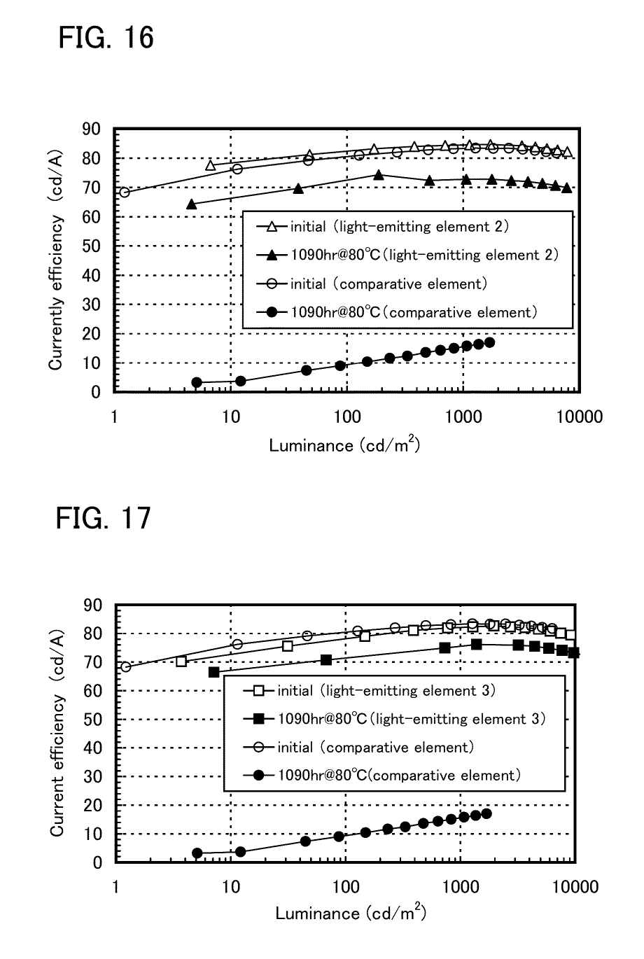

FIG. 16 and FIG. 17 show results of measuring current efficiencies of the light-emitting elements preserved at 80° C. for 1090 hours. FIG. 16 shows comparison between the light-emitting element 2 (2mDBTBPDBq-II; including two benzene rings) and the comparative light-emitting element (2mDBTPDBq-II; including one benzene ring). FIG. 17 shows comparison between the light-emitting element 3 (2mDBTPDBq-III; including three benzene rings) and the comparative light-emitting element (2mDBTPDBq-II; including one benzene ring).

As shown in these measurement results, deterioration of the current efficiencies of the light-emitting element 2 and the light-emitting element 3 is very small even after preservation at 80° C. for longer than 1000 hours. On the other hand, the current efficiency of the comparative light-emitting element is greatly deteriorated, which indicates current leakage.

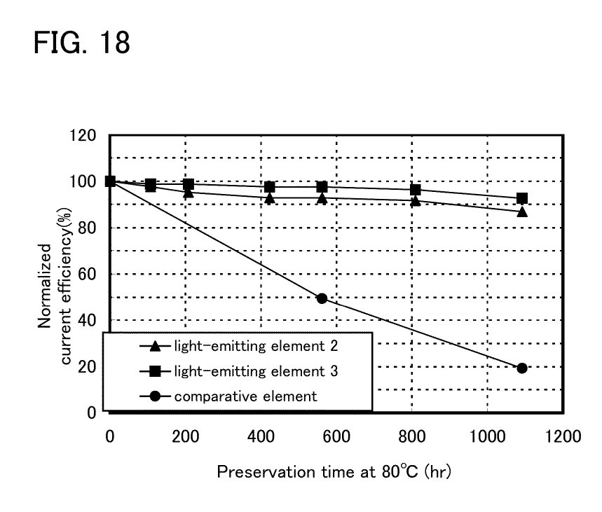

FIG. 18 shows preservation test results in this example. In FIG. 18, the horizontal axis represents the preservation time at 80° C. and the vertical axis indicates normalized value of the current efficiency of each element at a luminance of 1000 [cd/m²] with a current efficiency before the preservation test regarded as 100%. These results show that the behavior of the comparative light-emitting element including 2mDBTPDBq-II including only one benzene ring is extremely different from those of the light-emitting elements 2 and 3 each including two or more benzene rings in the high-temperature preservation test. In other words, characteristics of the comparative light-emitting element are largely deteriorated while the characteristics of the light-emitting elements 2 and 3 are hardly deteriorated.

A compound including one dibenzo[f,h]quinoxaline ring, one ring having a hole-transport skeleton, and benzene rings, such as the heterocyclic compound of an embodiment of the present invention, can be formed into a uniform film by vacuum evaporation, so that it is suitable for formation by vacuum evaporation. However, since the glass transition temperature (Tg) had not been observed, it had not been known which part of a skeleton of the compound had contributed to heat resistance. In other words, it had been difficult to determine how much Tg depends on difference in the number of benzene rings. However, as disclosed in this invention, in the case of a structure of the heterocyclic compound of an embodiment of the present invention, an obvious difference was found in heat resistance of the film of the compound having one benzene ring and the films of the compounds having two or more benzene rings. Thus, the heterocyclic compound of an embodiment of the present invention includes a unique number of benzene rings, thereby having higher heat resistance than the conventional heterocyclic compound.

### Reference Synthesis Example 1

This example gives descriptions of an example of a method of synthesizing 2-[3′-(dibenzothiophen-4-yl)biphenyl-3-yl]dibenzo[f,h]quinoxaline (abbreviation: 2mDBTBPDBq-II) represented by the following structural formula (103), which was used in Example 4.

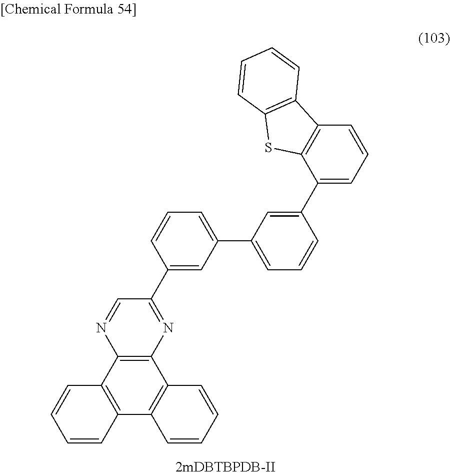 [SMILES: c1cc(-c2cccc(-c3cccc4c3sc3ccccc34)c2)cc(-c2cnc3c4ccccc4c4ccccc4c3n2)c1]

### Synthesis of 2mDBTBPDBq-II (abbreviation)

A synthesis scheme of 2mDBTBPDBq-II (abbreviation) is illustrated in (E-1).

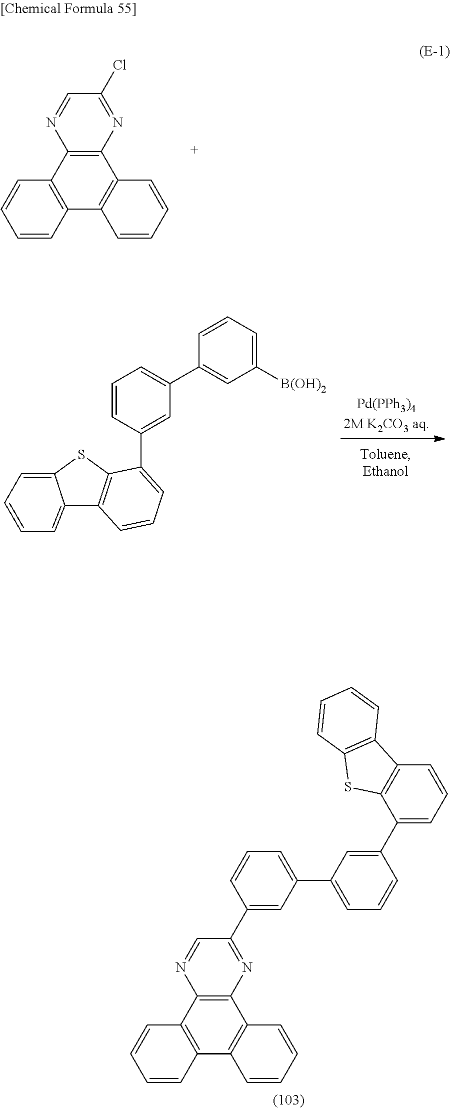

In a 200 mL three-neck flask were put 0.83 g (3.2 mmol) of 2-chlorodibenzo[f,h]quinoxaline, 1.3 g (3.5 mmol) of 3′-(dibenzothiophen-4-yl)-3-biphenylboronic acid, 40 mL of toluene, 4 mL of ethanol, and 5 mL of a 2M aqueous solution of potassium carbonate. This mixture was degassed by stirring under reduced pressure, and the air in the flask was replaced with nitrogen. To this mixture was added 80 mg (70 μmol) of tetrakis(triphenylphosphine)palladium(0). This mixture was stirred at 80° C. for 16 hours under a nitrogen stream. After a predetermined time had elapsed, the precipitated solid was separated by filtration to give a yellow solid. Ethanol was added to this solid, followed by irradiation with ultrasonic waves. The mixture was suction filtered to give a solid. The obtained solid was dissolved in toluene, the toluene solution was suction filtered through alumina and Celite (produced by Wako Pure Chemical Industries, Ltd., Catalog No. 531-16855), and the filtrate was concentrated to give a yellow solid. Further, this solid was recrystallized with toluene to give 1.1 g of a yellow powder in a yield of 57%.

By a train sublimation method, 1.1 g of the obtained yellow powder was purified. In the purification, the yellow powder was heated at 300° C. under a pressure of 6.2 Pa with a flow rate of an argon gas at 15 mL/min. Through the purification, 0.80 g of a target yellow powder was obtained in a yield of 73%.

This compound was identified as 2mDBTBPDBq-II (abbreviation), which was the object of the synthesis, by nuclear magnetic resonance (NMR) spectroscopy.

¹H NMR data of the obtained substance is shown below. ¹H NMR (CDCl₃, 300 MHz): δ=7.46-7.50 (m, 2H), 7.61 (d, J=4.5 Hz, 2H), 7.67-7.89 (m, 10H), 8.17-8.24 (m, 3H), 8.35 (d, J=8.1 Hz, 1H), 8.65-8.70 (m, 3H), 9.24-9.27 (m, 1H), 9.44-9.48 (m, 2H).

Next, a result of analyzing 2mDBTBPDBq-II (abbreviation) obtained in Reference Synthesis Example 1 by liquid chromatography mass spectrometry (LC/MS) is shown below.

The LC/MS was carried out with Acquity UPLC (produced by Waters Corporation) and Xevo G2 T of MS (produced by Waters Corporation).

In the MS, ionization was carried out by an electrospray ionization (ESI) method. At this time, the capillary voltage and the sample cone voltage were set to 3.0 kV and 30 V, respectively, and detection was performed in a positive mode.

A component which underwent the ionization under the above-described conditions was collided with an argon gas in a collision cell to dissociate into a plurality of product ions. Energy (collision energy) for the collision with argon was 50 eV.

The mass range for the measurement was m/z=100 to 1200.

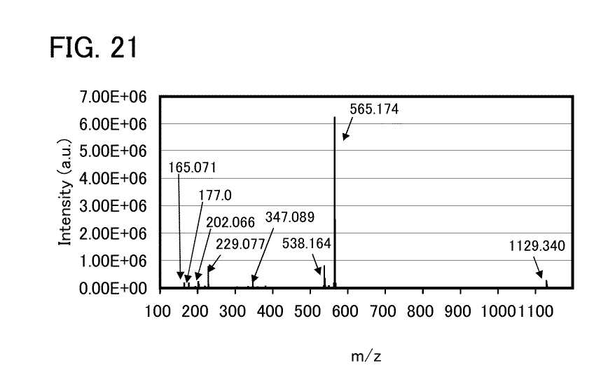

FIG. 21 shows the measurement result. The result in FIG. 21 shows that product ions of 2mDBTBPDBq-II (abbreviation), which is the heterocyclic compound of an embodiment of the present invention represented by the structural formula (103), are detected mainly around m/z=347 and m/z=229.

Note that the result in FIG. 21 shows characteristics derived from 2mDBTBPDBq-II (abbreviation) and therefore can be regarded as important data for identifying 2mDBTBPDBq-II (abbreviation) contained in the mixture.

The product ion around m/z=538 is assumed to be a cation in a state where one C atom and one N atom are detached from the dibenzo[f,h]quinoxaline ring of the compound represented by the structural formula (103), which is one feature of the heterocyclic compound of an embodiment of the present invention. Further, the product ion around m/z=229 is assumed to be a cation of a diazatriphenylenyl group such as dibenzo[f,h]quinoxaline. Moreover, the product ions around m/z=202, m/z=177, and m/z=165 are also detected at the same time. Therefore, it is indicated that 2mDBTBPDBq-II (abbreviation) which is the heterocyclic compound of an embodiment of the present invention includes a dibenzo[f,h]quinoxaline ring.

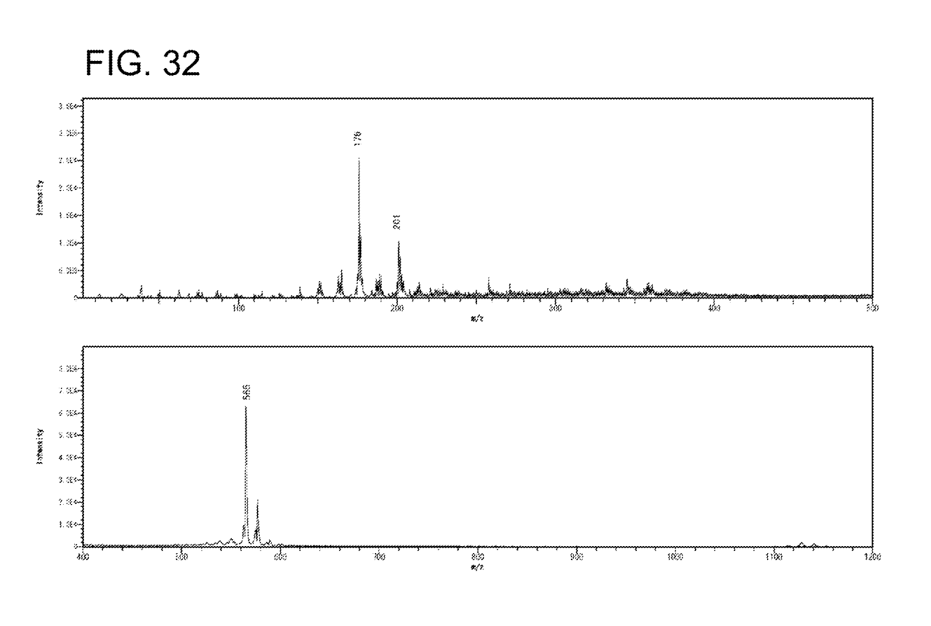

Further, 2mDBTBPDBq-II (abbreviation), which is an embodiment of the present invention, was measured with a time-of-flight secondary ion mass spectrometer (TOF-SIMS); FIG. 32 shows the obtained qualitative spectrum in the case of a positive ion.

TOF.SIMS 5 (produced by ION-TOF GmbH) was used, and Bi₃⁺⁺ was used as a primary ion source. Note that irradiation with the primary ions was performed in a pulsed manner with a pulse width of 7 nm to 12 nm. The irradiation amount was greater than or equal to 8.2×10¹⁰ ions/cm² and less than or equal to 6.7×10¹¹ ions/cm² (less than or equal to 1×10¹² ions/cm²), the acceleration voltage was 25 keV, and the current value was 0.2 pA. A powder of 2mDBTBPDBq-II (abbreviation) was the sample used for the measurement.

The result of analysis by TOF-SIMS (positive ion) in FIG. 32 shows that product ions of 2mDBTBPDBq-II (abbreviation) (m/z=564.17), which is the heterocyclic compound of an embodiment of the present invention represented by the structural formula (103), are detected mainly around m/z=565, m/z=201, and m/z=176. Here, the expression “around” indicates that difference in values of product ions, which change depending on whether a hydrogen ion or an isotope is present or not, is allowable. Since the product ions shown in the result in FIG. 32 are similar to the product ions of 2mDBTBPDBq-II (abbreviation) in FIG. 21, which were detected by the MS analysis (positive ion), the result of the measurement by TOF-SIMS can also be regarded as important data for identifying 2mDBTBPDBq-II (abbreviation) contained in the mixture.

### Reference Synthesis Example 2

This example gives descriptions of an example of a method of synthesizing 2-[3-(2,8-diphenyldibenzothiophen-4-yl)phenyl]dibenzo[f,h]quinoxaline (abbreviation: 2mDBTPDBq-III) represented by the following structural formula (113), which was used in Example 4.

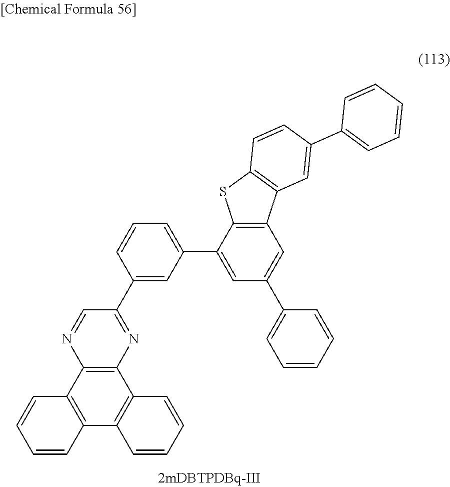 [SMILES: c1ccc(-c2ccc3sc4c(-c5cccc(-c6cnc7c8ccccc8c8ccccc8c7n6)c5)cc(-c5ccccc5)cc4c3c2)cc1]

### Synthesis of 2mDBTPDBq-III (abbreviation)

A synthesis scheme of 2mDBTPDBq-III (abbreviation) is illustrated in (F-1).

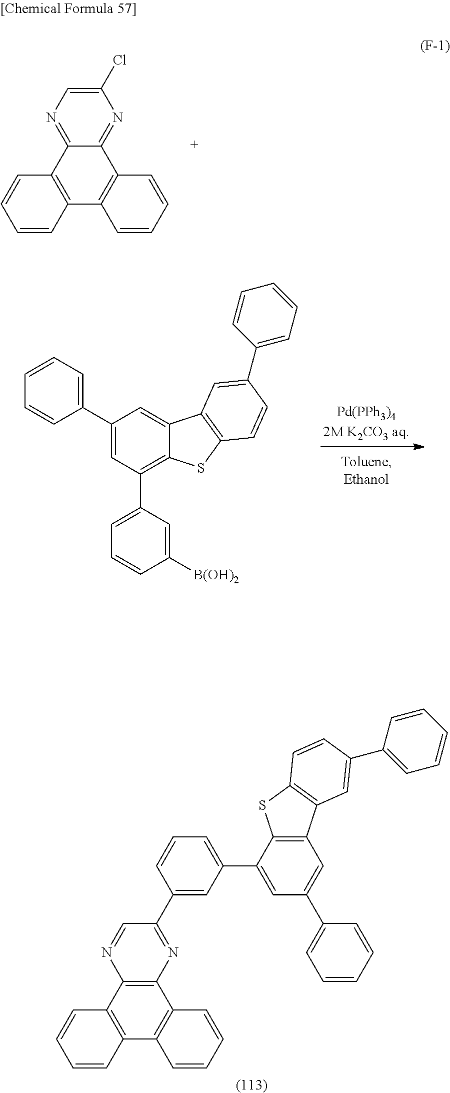 [SMILES: Clc1cnc2c3ccccc3c3ccccc3c2n1 ; OB(O)c1cccc(-c2cc(-c3ccccc3)cc3c2sc2ccc(-c4ccccc4)cc23)c1 ; c1ccc(-c2ccc3sc4c(-c5cccc(-c6cnc7c8ccccc8c8ccccc8c7n6)c5)cc(-c5ccccc5)cc4c3c2)cc1 ; "(113)"]

In a 100 mL three-neck flask were put 0.40 g (1.5 mmol) of 2-chlorodibenzo[f,h]quinoxaline, 0.68 g (1.5 mmol) of 3-(2,8-diphenyldibenzothiophen-4-yl)phenylboronic acid, 15 mL of toluene, 2.0 mL of ethanol, and 1.5 mL of a 2M aqueous solution of potassium carbonate. This mixture was degassed by stirring under reduced pressure, and the air in the flask was replaced with nitrogen. To this mixture was added 51 mg (43 μmol) of tetrakis(triphenylphosphine)palladium(0). This mixture was stirred at 80° C. for 4 hours under a nitrogen stream. After a predetermined time had elapsed, water was added to the obtained mixture, and organic substances were extracted from the aqueous layer with toluene. The extracted solution and the organic layer were combined, washed with saturated saline, and dried with magnesium sulfate. The obtained mixture was gravity filtered, and the filtrate was concentrated to give a solid. The obtained solid was dissolved in toluene, and the toluene solution was suction filtered through alumina, Florisil (produced by Wako Pure Chemical Industries, Ltd., Catalog No. 540-00135), and Celite (produced by Wako Pure Chemical Industries, Ltd., Catalog No. 531-16855), and the obtained filtrate was concentrated to give a solid. The obtained solid was washed with toluene, methanol was added to this solid, and the methanol suspension was irradiated with ultrasonic waves. A solid was collected by suction filtration to give 0.60 g of a target white powder in a yield of 61%.

By a train sublimation method, 0.59 g of the obtained white powder was purified. In the purification, the white powder was heated at 330° C. under a pressure of 2.7 Pa with a flow rate of an argon gas at 5 mL/min. Through the purification, 0.54 g of a target white powder was obtained in a yield of 90%.

This compound was identified as 2mDBTPDBq-III (abbreviation), which was the object of the synthesis, by nuclear magnetic resonance (NMR) spectroscopy.

¹H NMR data of the obtained substance is shown below. ¹H NMR (CDCl₃, 300 MHz): δ=7.37-7.55 (m, 6H), 7.73-7.84 (m, 10H), 7.90-7.98 (m, 3H), 8.44-8.48 (m, 3H), 8.65 (dd, J=7.8 Hz, 1.5 Hz, 2H), 8.84-8.85 (m, 1H), 9.27 (dd, J=7.2 Hz, 2.7 Hz, 1H), 9.46 (dd, J=7.8 Hz, 2.1 Hz, 1H), 9.51 (s, 1H).

Next, results of analyzing 2mDBTPDBq-III (abbreviation) obtained in Reference Synthesis Example 2 by liquid chromatography mass spectrometry (LC/MS) are shown below.

The LC/MS was carried out with Acquity UPLC (produced by Waters Corporation) and Xevo G2 T of MS (produced by Waters Corporation).

In the MS, ionization was carried out by an electrospray ionization (ESI) method. At this time, the capillary voltage and the sample cone voltage were set to 3.0 kV and 30 V, respectively, and detection was performed in a positive mode.

A component which underwent the ionization under the above-described conditions was collided with an argon gas in a collision cell to dissociate into a plurality of product ions. Energy (collision energy) for the collision with argon was 50 eV and 70 eV. The mass range for the measurement was m/z=100 to 1200.

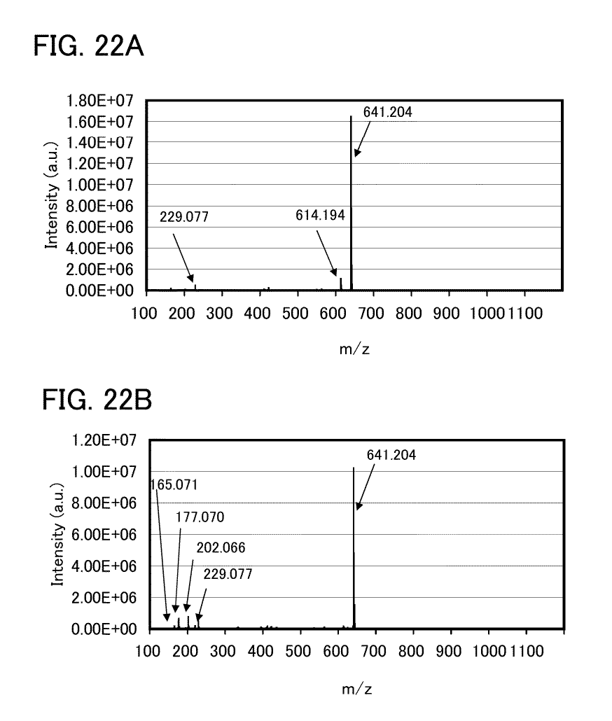

FIGS. 22A and 22B show the measurement results. Note that FIG. 22A shows the case of 50 eV and FIG. 22B shows the case of 70 eV. The result in FIG. 22B shows that product ions of 2mDBTPDBq-III (abbreviation), which is the heterocyclic compound of an embodiment of the present invention represented by the structural formula (113), are detected mainly around m/z=229, m/z=202, and m/z=177.

Note that the result in FIG. 22B shows characteristics derived from 2mDBTPDBq-III (abbreviation) and therefore can be regarded as important data for identifying 2mDBTPDBq-III (abbreviation) contained in the mixture.

The product ion around m/z=614 in FIG. 22A is assumed to be a cation in a state where one C atom and one N atom are detached from the dibenzo[f,h]quinoxaline ring of the compound represented by the structural formula (113), which is one feature of the heterocyclic compound of an embodiment of the present invention. Further, the product ion around m/z=229 is assumed to be a cation of a diazatriphenylenyl group such as dibenzo[f,h]quinoxaline. Moreover, the product ions around m/z=202, m/z=177, and m/z=165 are also detected at the same time. Therefore, it is indicated that 2mDBTPDBq-III (abbreviation) which is the heterocyclic compound of an embodiment of the present invention includes a dibenzo[f,h]quinoxaline ring.

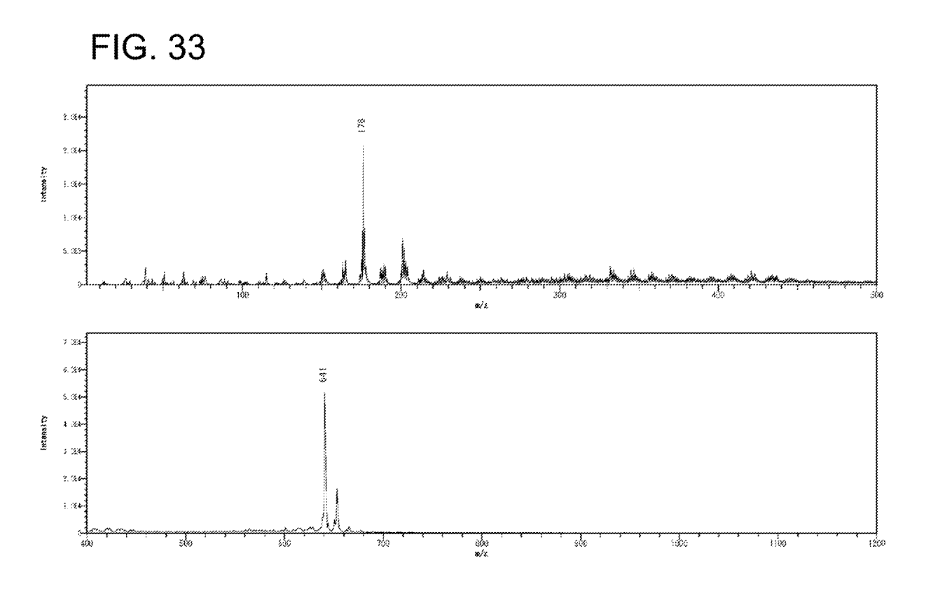

Further, 2mDBTPDBq-III (abbreviation), which is an embodiment of the present invention, was measured with a time-of-flight secondary ion mass spectrometer (TOF-SIMS); FIG. 33 shows the obtained qualitative spectrum in the case of a positive ion.

TOF.SIMS 5 (produced by ION-TOF GmbH) was used, and Bi₃⁺⁺ was used as a primary ion source. Note that irradiation with the primary ions was performed in a pulsed manner with a pulse width of 7 nm to 12 nm. The irradiation amount was greater than or equal to 8.2×10¹⁰ ions/cm² and less than or equal to 6.7×10¹¹ ions/cm² (less than or equal to 1×10¹² ions/cm²), the acceleration voltage was 25 keV, and the current value was 0.2 pA. A powder of 2mDBTPDBq-III (abbreviation) was the sample used for the measurement.

The result of analysis by TOF-SIMS (positive ion) in FIG. 33 shows that a product ion of 2mDBTPDBq-III (abbreviation) (m/z=640.2), which is the heterocyclic compound of an embodiment of the present invention represented by the structural formula (113), is detected mainly around m/z=176. Here, the expression “around” indicates that difference in values of product ions, which change depending on whether a hydrogen ion or an isotope is present or not, is allowable. Since the product ions shown in the result in FIG. 33 are similar to the product ions of 2mDBTPDBq-III (abbreviation) in FIGS. 22A and 22B, which were detected by the MS analysis (positive ion), the result of the measurement by TOF-SIMS can also be regarded as important data for identifying 2mDBTPDBq-III (abbreviation) contained in the mixture.

### Reference Synthesis Example 3

This example gives descriptions of an example of a method of synthesizing 2-[3-(dibenzothiophen-4-yl)phenyl]dibenzo[f, h]quinoxaline (abbreviation: 2mDBTPDBq-II) represented by the following structural formula (600).

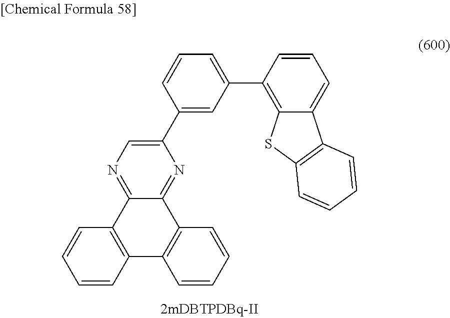 [SMILES: c1cc(-c2cnc3c4ccccc4c4ccccc4c3n2)cc(-c2cccc3c2sc2ccccc23)c1]

### Synthesis of 2mDBTPDBq-II (abbreviation)

A synthesis scheme of 2mDBTPDBq-II (abbreviation) is illustrated in (G-1).

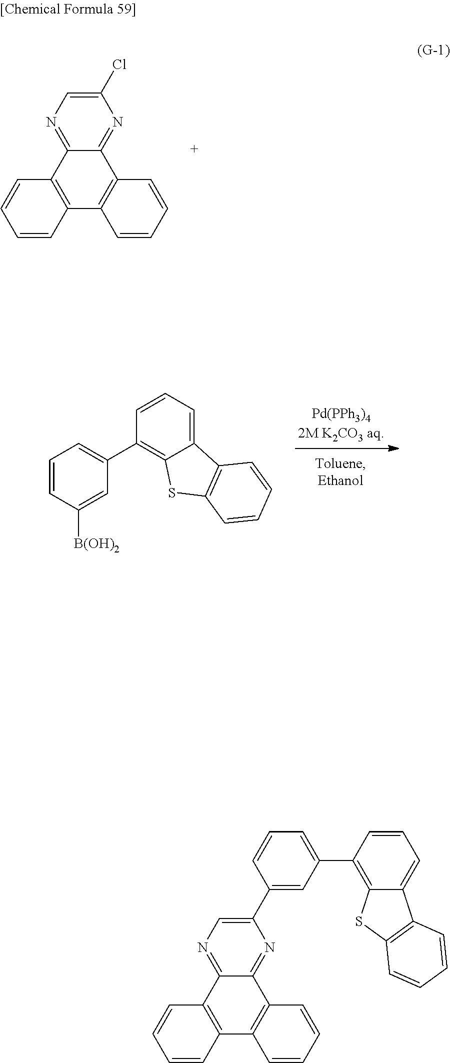 [SMILES: Clc1cnc2c3ccccc3c3ccccc3c2n1 ; OB(O)c1cccc(-c2cccc3c2sc2ccccc23)c1 ; c1cc(-c2cnc3c4ccccc4c4ccccc4c3n2)cc(-c2cccc3c2sc2ccccc23)c1]

In a 2 L three-neck flask were put 5.3 g (20 mmol) of 2-chlorodibenzo[f,h]quinoxaline, 6.1 g (20 mmol) of 3-(dibenzothiophen-4-yl)phenylboronic acid, 460 mg (0.4 mmol) of tetrakis(triphenylphosphine)palladium(0), 300 mL of toluene, 20 mL of ethanol, and 20 mL of a 2M aqueous solution of potassium carbonate. This mixture was degassed by stirring under reduced pressure, and the air in the flask was replaced with nitrogen. This mixture was stirred at 100° C. for 7.5 hours under a nitrogen stream. After cooled to room temperature, the obtained mixture was filtered to give a white residue. The obtained residue was washed with water and ethanol in this order, and then dried. The obtained solid was dissolved in about 600 mL of hot toluene, followed by suction filtration through Celite (produced by Wako Pure Chemical Industries, Ltd., Catalog No. 531-16855) and Florisil (produced by Wako Pure Chemical Industries, Ltd., Catalog No. 540-00135), whereby a clear colorless filtrate was obtained. The obtained filtrate was concentrated and purified by silica gel column chromatography. The chromatography was carried out using hot toluene as a developing solvent. Acetone and ethanol were added to the solid obtained here, followed by irradiation with ultrasonic waves. Then, the generated suspended solid was filtered and the obtained solid was dried to give 7.85 g of a target white powder in a yield of 80%.

The above produced substance was relatively soluble in hot toluene, but is a material that is easy to precipitate when cooled. Further, the substance was poorly soluble in other organic solvents such as acetone and ethanol. Hence, the utilization of these different degrees of solubility resulted in a high-yield synthesis by a simple method as above. Specifically, after the reaction finished, the mixture was returned to room temperature and the precipitated solid was collected by filtration, whereby most impurities were able to be easily removed. Further, by the column chromatography with hot toluene as a developing solvent, the produced substance, which is easy to precipitate, was able to be readily purified.

By a train sublimation method, 4.0 g of the obtained white powder was purified. In the purification, the white powder was heated at 300° C. under a pressure of 5.0 Pa with a flow rate of an argon gas at 5 mL/min. Through the purification, 3.5 g of a target white powder was obtained in a yield of 88%.

This compound was identified as 2mDBTPDBq-II (abbreviation), which was the object of the synthesis, by nuclear magnetic resonance (NMR) spectroscopy.

¹H NMR data of the obtained substance is shown below. ¹H NMR (CDCl₃, 300 MHz): δ (ppm)=7.45-7.52 (m, 2H), 7.59-7.65 (m, 2H), 7.71-7.91 (m, 7H), 8.20-8.25 (m, 2H), 8.41 (d, J=7.8 Hz, 1H), 8.65 (d, J=7.5 Hz, 2H), 8.77-8.78 (m, 1H), 9.23 (dd, J=7.2 Hz, 1.5 Hz, 1H), 9.42 (dd, J=7.8 Hz, 1.5 Hz, 1H), 9.48 (s, 1H).

### Example 5

This example gives descriptions of a light-emitting element 4 in which 2-{3-[3-(2,8-diphenyldibenzothiophen-4-yl)phenyl]phenyl}dibenzo[f,h]quinoxaline (abbreviation: 2mDBTBPDBq-III) (structural formula (418)), which is a heterocyclic compound of an embodiment of the present invention, is used for part of a light-emitting layer, with reference to FIG. 11 which is used for explanation of the light-emitting element 1 in Example 3. Chemical formulae of materials used in this example are shown below.

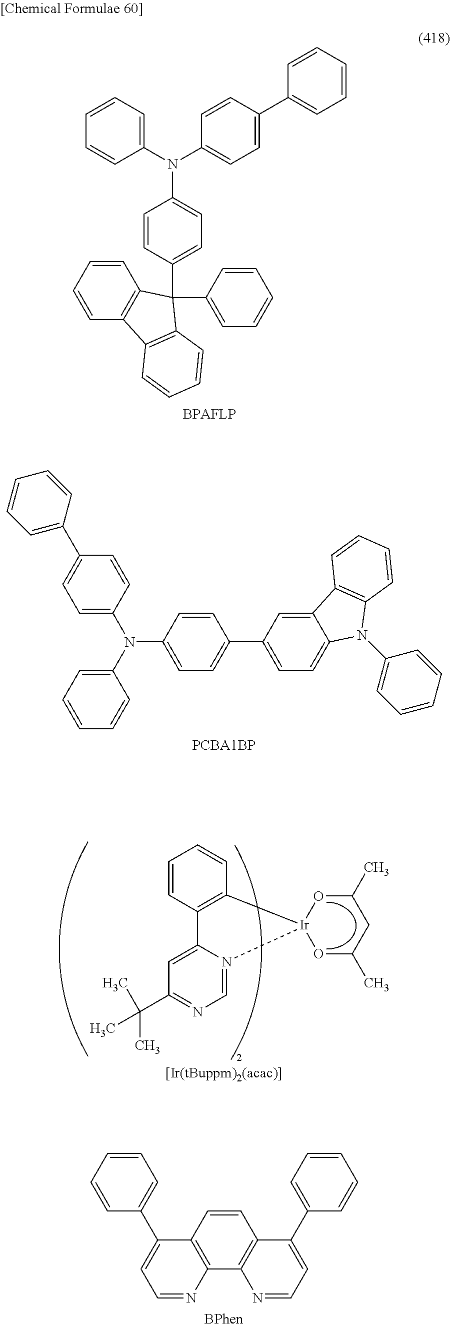

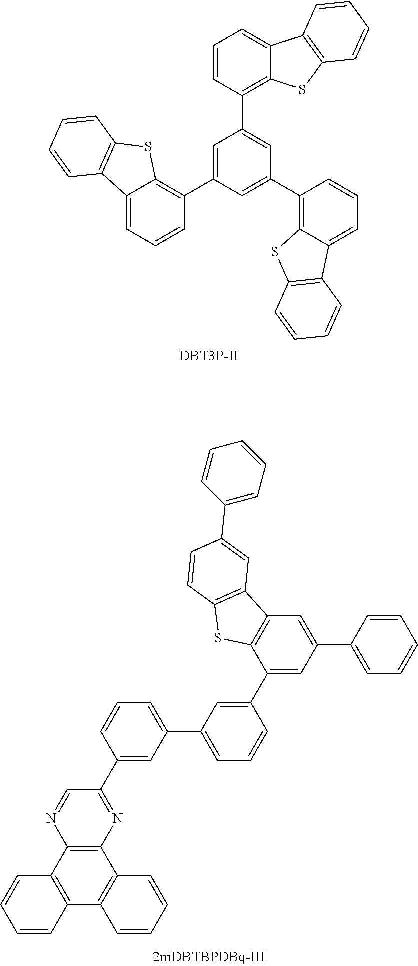 [SMILES: c1ccc2c(c1)sc1c(-c3cc(-c4cccc5c4sc4ccccc45)cc(-c4cccc5c4sc4ccccc45)c3)cccc12 ; "DBT3P-II" ; c1ccc(-c2ccc3sc4c(-c5cccc(-c6cccc(-c7cnc8c9ccccc9c9ccccc9c8n7)c6)c5)cc(-c5ccccc5)cc4c3c2)cc1]

### <Fabrication of Light-Emitting Element 4>

First, indium tin oxide containing silicon oxide (ITSO) was deposited over a glass substrate 1100 by a sputtering method, so that a first electrode 1101 which functions as an anode was formed. The thickness was 110 nm and the electrode area was 2 mm×2 mm.

Then, as pretreatment for forming the light-emitting element over the substrate 1100, UV ozone treatment was performed for 370 seconds after washing of a surface of the substrate with water and baking that was performed at 200° C. for 1 hour.

After that, the substrate was transferred into a vacuum evaporation apparatus where the pressure had been reduced to approximately 10⁻⁴ Pa, and was subjected to vacuum baking at 170° C. for 30 minutes in a heating chamber of the vacuum evaporation apparatus, and then the substrate 1100 was cooled down for about 30 minutes.

Next, the substrate 1100 was fixed to a holder provided in the vacuum evaporation apparatus so that the surface of the substrate 1100 over which the first electrode 1101 was formed faced downward. In this example, a case is described in which a hole-injection layer 1111, a hole-transport layer 1112, a light-emitting layer 1113, an electron-transport layer 1114, and an electron-injection layer 1115 which are included in an EL layer 1102 are sequentially formed by a vacuum evaporation method.

After reducing the pressure of the vacuum evaporation apparatus to 10⁻⁴ Pa, 1,3,5-tri(dibenzothiophen-4-yl)benzene (abbreviation: DBT3P-II) and molybdenum(VI) oxide were co-evaporated with a mass ratio of DBT3P-II (abbreviation) to molybdenum oxide being 4:2, whereby the hole-injection layer 1111 was formed over the first electrode 1101. The thickness of the hole-injection layer 1111 was 40 nm. Note that the co-evaporation is an evaporation method in which some different substances are evaporated from some different evaporation sources at the same time.

Then, 4-phenyl-4′-(9-phenylfluoren-9-yl)triphenylamine (abbreviation: BPAFLP) was evaporated to a thickness of 20 nm, so that the hole-transport layer 1112 was formed.

Next, the light-emitting layer 1113 was formed over the hole-transport layer 1112. Co-evaporated were 2-{3-[3-(2,8-diphenyldibenzothiophen-4-yl)phenyl]phenyl}dibenzo[f,h]quinoxaline (abbreviation: 2mDBTBPDBq-III), 4-phenyl-4′-(9-phenyl-9H-carbazol-3-yl)triphenylamine (abbreviation: PCBA1BP), and (acetylacetonato)bis(6-tert-butyl-4-phenylpyrimidinato)iridium(III) (abbreviation: [Ir(tBuppm)₂(acac)]) with a mass ratio of 2mDBTBPDBq-III (abbreviation) to PCBA1BP (abbreviation) and [Ir(tBuppm)₂(acac)] (abbreviation) being 0.8:0.2:0.05. The thickness of the light-emitting layer 1113 was 40 nm. Thus, the light-emitting layer 1113 was formed.

Then, 2mDBTBPDBq-III (abbreviation) was evaporated to a thickness of 10 nm over the light-emitting layer 1113 and bathophenanthroline (abbreviation: Bphen) was evaporated to a thickness of 20 nm, whereby the electron-transport layer 1114 having a stacked structure was formed. Furthermore, lithium fluoride was evaporated to a thickness of 1 nm over the electron-transport layer 1114, whereby the electron-injection layer 1115 was formed.

Finally, aluminum was evaporated to a thickness of 200 nm over the electron-injection layer 1115 to form a second electrode 1103 serving as a cathode; thus, the light-emitting element 4 was obtained. Note that in all the above evaporation steps, evaporation was performed by a resistance-heating method.

An element structure of the light-emitting element 4 obtained as described above is shown in Table 4.

TABLE 4

| | | Hole- | Hole- | Light- | Electron- | | Electron- | |
|---|---|---|---|---|---|---|---|---|
| | First | injection | transport | emitting | transport | | injection | Second |
| | Electrode | Layer | Layer | Layer | Layer | | Layer | Electrode |
| Light- | ITSO | DBT3P-II: | BPAFLP | *4 | **4 | Bphen | LiF | Al |
| emitting | (110 nm) | MoOx | (20 nm) | | | (20 nm) | (1 nm) | (200 nm) |
| Element 4 | | (4:2, 40 nm) | | | | | | |

*4 2mDBTBPDBq-III:PCBA1BP:[Ir(tBuppm)₂(acac)] (0.8:0.2:0.05, 40 nm)
**4 2mDBTBPDBq-III (10 nm)

Further, the manufactured light-emitting element 4 was sealed in a glove box containing a nitrogen atmosphere so as not to be exposed to the air (a sealant was applied onto an outer edge of the element and heat treatment was performed at 80° C. for 1 hour at the time of sealing).

### <Operation Characteristics of Light-Emitting Element 4>

Operation characteristics of the manufactured light-emitting element 4 were measured. Note that the measurement was carried out at room temperature (under an atmosphere in which the temperature was kept at 25° C.).

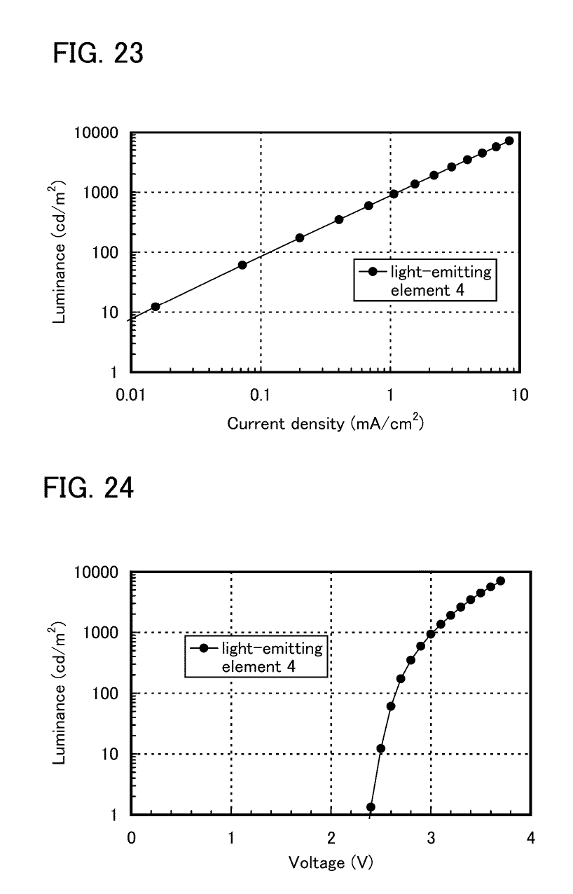

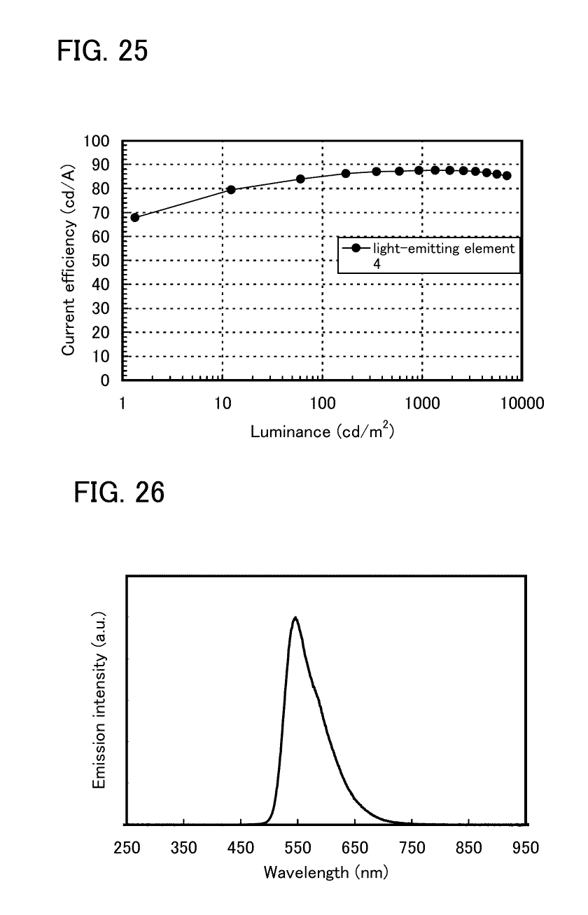

FIG. 23 shows luminance versus current density characteristics, FIG. 24 shows luminance versus voltage characteristics, and FIG. 25 shows current efficiency versus luminance characteristics of the light-emitting element 4.

FIG. 25 suggests that the light-emitting element 4 in which the heterocyclic compound of an embodiment of the present invention is used for part of the light-emitting layer as a host material has low power consumption and high efficiency.

Table 5 below shows initial values of main characteristics of the light-emitting element 4 at a luminance of about 1000 cd/m².

TABLE 5

| | | | | | | | | External |
|---|---|---|---|---|---|---|---|---|
| | | | Current | | | Current | Power | Quantum |
| | Voltage | Current | Density | Chromaticity | Luminance | Efficiency | Efficiency | Efficiency |
| | (V) | (mA) | (mA/cm²) | (x, y) | (cd/m²) | (cd/A) | (lm/W) | (%) |
| Light- | 3.0 | 0.043 | 1.1 | (0.43, 0.56) | 930 | 87 | 92 | 24 |
| emitting | | | | | | | | |
| Element 4 | | | | | | | | |

The above results in Table 5 also suggest that the light-emitting element 4 manufactured in this example has high luminance and high current efficiency.

FIG. 26 shows an emission spectrum when a current at a current density of 0.1 mA/cm² was supplied to the light-emitting element 4. FIG. 26 shows that the emission spectrum of the light-emitting element 4 has a peak at around 544 nm, which indicates that the emission spectrum is derived from emission of [Ir(tBuppm)₂(acac)] (abbreviation) included in the light-emitting layer 1113.

Thus, it was found that 2mDBTBPDBq-III (abbreviation) has a high T1 level and can be used for a host material or a carrier-transport material in a light-emitting element which exhibits phosphorescence in a visible light region (wavelength longer than or equal to that of blue light).

### <Heat-Resisting Property of Light-Emitting Element 4>

Heat-resistance test was conducted on the manufactured light-emitting element 4. The evaluation was performed in such a manner that the light-emitting element 4 was preserved in a constant temperature bath kept at 100° C. for a predetermined period and then current efficiency was measured. Note that the current efficiency was measured at room temperature (in an atmosphere kept at 25° C.) after the light-emitting element 4 was taken out of the constant temperature bath.

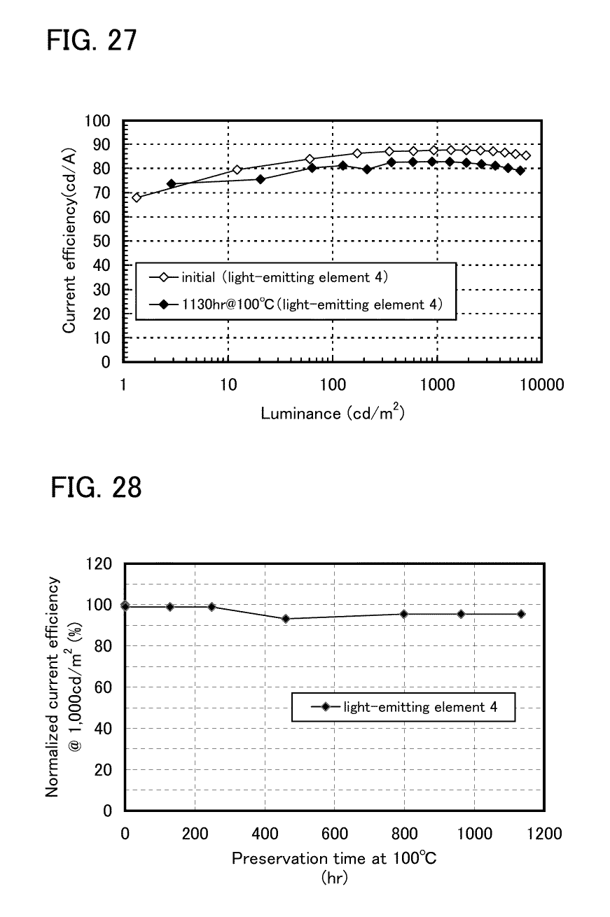

FIG. 27 shows a result of measuring current efficiency of the light-emitting element 4 preserved at 100° C. for 1130 hours.

As shown in this measurement result, deterioration of the current efficiency of the light-emitting element 4 is very small even after preservation at 100° C. for longer than 1000 hours.

FIG. 28 shows normalized current efficiency versus preservation time at 100° C., which is the result of preservation test in this example. In FIG. 28, the horizontal axis represents preservation time at 100° C. and the vertical axis represents normalized value of the current efficiency of the light-emitting element 4 at a luminance of 1000 [cd/m²] with a current efficiency before the preservation test regarded as 100%. This result shows that the characteristics of the light-emitting element 4 including 2mDBTBPDBq-III (abbreviation) including four benzene rings are hardly deteriorated.

A compound including one dibenzo[f,h]quinoxaline ring, one ring having a hole-transport skeleton, and benzene rings, such as the heterocyclic compound of an embodiment of the present invention, can be formed into a uniform film by vacuum evaporation, so that it is suitable for formation by vacuum evaporation. However, since the glass transition temperature (Tg) had not been observed, it had not been known which part of a skeleton of the compound had contributed to heat resistance. In other words, it had been difficult to determine how much Tg depends on difference in the number of benzene rings. However, as disclosed in this invention, in the case of a structure of the heterocyclic compound of an embodiment of the present invention, an obvious difference was found in heat resistance of the film of the compound having one benzene ring and the films of the compounds having two or more benzene rings. Thus, the heterocyclic compound of an embodiment of the present invention includes a unique number of benzene rings, thereby having higher heat resistance than the conventional heterocyclic compound.

### Example 6

### Synthesis Example 3

This example gives descriptions of a method for synthesizing 2-{3-[3-(6-phenyldibenzothiophen-4-yl)phenyl]phenyl}dibenzo[f,h]quinoxaline (abbreviation: 2mDBTBPDBq-IV), which is a heterocyclic compound of an embodiment of the present invention represented by the structural formula (131) in Embodiment 1. A structure of 2mDBTBPDBq-W (abbreviation) is shown below.

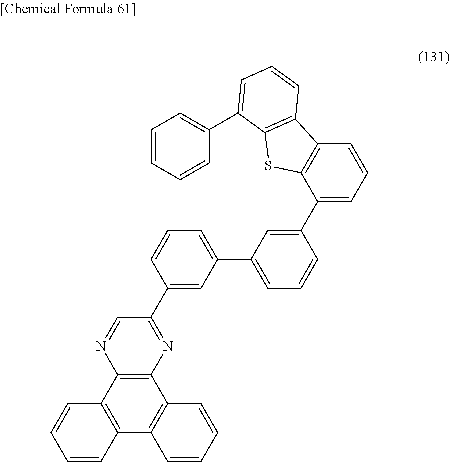 [SMILES: c1ccc(-c2cccc3c2sc2c(-c4cccc(-c5cccc(-c6cnc7c8ccccc8c8ccccc8c7n6)c5)c4)cccc23)cc1]

### Step 1: Synthesis of 4-(3-bromophenyl)-6-phenyldibenzothiophene

A synthesis scheme of 4-(3-bromophenyl)-6-phenyldibenzothiophene is illustrated in (H-1).

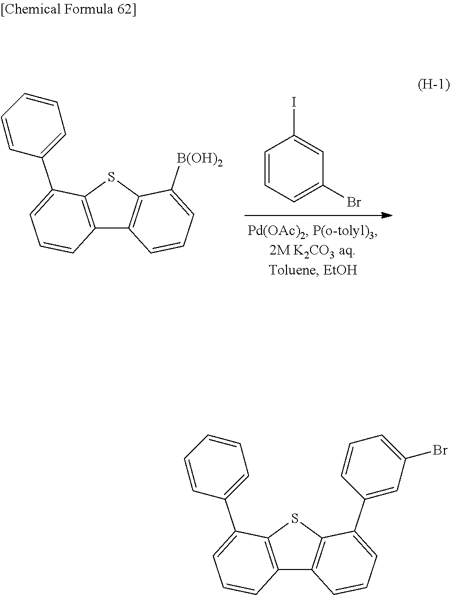 [SMILES: OB(O)c1cccc2c1sc1c(-c3ccccc3)cccc12 ; Brc1cccc(I)c1 ; Brc1cccc(-c2cccc3c2sc2c(-c4ccccc4)cccc23)c1]

In a 300 mL three-neck flask were put 7.00 g (23.0 mmol) of 6-phenyldibenzothiophen-4-ylboronic acid, 8.46 g (29.9 mmol) of 1-bromo-3-iodobenzene, 840 mg (2.8 mmol) of tris(2-methylphenyl)phosphine, 115 mL of toluene, 23 mL of ethanol, and 35 mL of a 2M aqueous solution of potassium carbonate. This mixture was degassed by stirring. Then, 155 mg (0.69 mmol) of palladium acetate was added to this mixture, and the mixture was heated and stirred at 80° C. for 4 hours under a nitrogen stream to be reacted. After the reaction, the aqueous layer of this mixture was extracted with toluene, and the extracted solution and the organic layer were combined and washed with saturated saline. The organic layer was dried with magnesium sulfate and the mixture was gravity filtrated after the drying.

The obtained filtrate was concentrated to an appropriate amount and then filtered through Celite and alumina. The filtrate was concentrated to give a brown oily substance. Hexane and acetonitrile were added to the oily substance for separation, and the hexane layer was concentrated to give 7.45 g of a white solid in a yield of 78.0%.

### Step 2: Synthesis of 3-(6-phenyldibenzothiophen-4-yl)phenylboronic acid

A synthesis scheme of 3-(6-phenyldibenzothiophen-4-yl)phenylboronic acid is illustrated in (H-2).

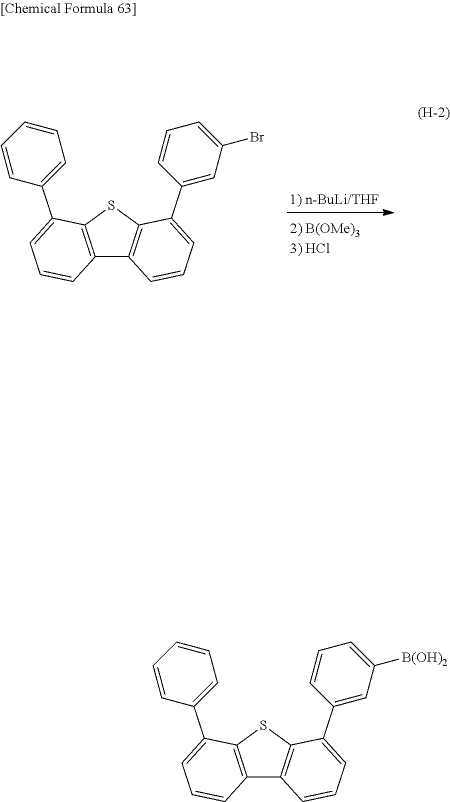 [SMILES: Brc1cccc(-c2cccc3c2sc2c(-c4ccccc4)cccc23)c1 ; OB(O)c1cccc(-c2cccc3c2sc2c(-c4ccccc4)cccc23)c1]

In a 300 mL flask was put 5.69 g (13.7 mmol) of 4-(3-bromophenyl)-6-phenyldibenzothiophene and the air in the flask was replaced with nitrogen. Then, 137 mL of tetrahydrofuran (THF) was added and this solution was cooled to −78° C. under a nitrogen stream. After the cooling, 9.4 mL (15.0 mmol) of n-butyllithium (a 1.6 mol/L hexane solution) was dripped to this solution with a syringe. After that, this solution was stirred at the same temperature for 2 hours. Then, 1.6 mL (16.4 mmol) of trimethyl borate was added to this solution and this mixture was stirred for 16 hours while its temperature was returned to room temperature. After the stirring, about 40 mL of 1M hydrochloric acid was added to this solution, followed by stirring for 2.5 hours. Then, the aqueous layer of this mixture was extracted with ethyl acetate, and the obtained extracted solution and the organic layer were combined and washed with a saturated aqueous solution of sodium hydrogen carbonate and saturated saline. The organic layer was dried with magnesium sulfate. After the drying, this mixture was subjected to gravity filtration and the filtrate was concentrated to give a brown oily substance. This oily substance was recrystallized with toluene/hexane to give 1.2 g of a target pale brown solid in a yield of 23%.

### Step 3: Synthesis of 2-{3-[3-(6-phenyldibenzothiophen-4-yl)phenyl]phenyl}dibenzo[f,h]quinoxaline (abbreviation: 2mDBTBPDBq-IV)

A synthesis scheme of 2mDBTBPDBq-IV (abbreviation) is illustrated in (H-3).

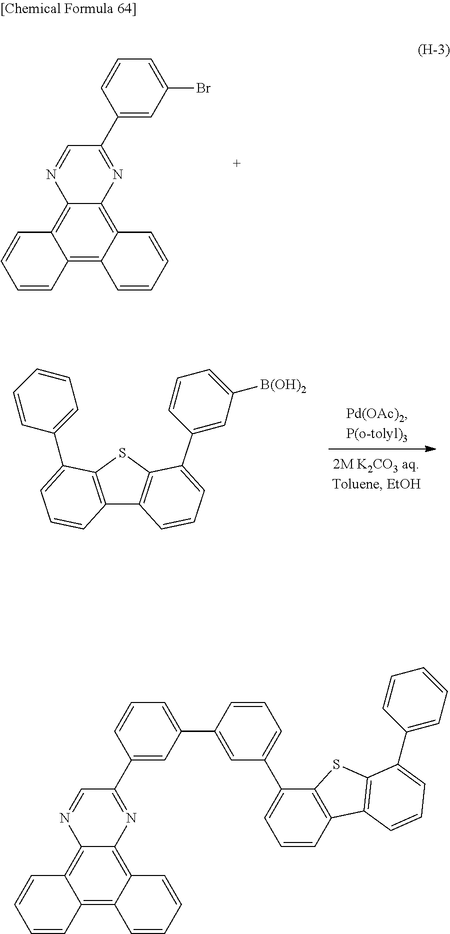 [SMILES: Brc1cccc(-c2cnc3c4ccccc4c4ccccc4c3n2)c1 ; OB(O)c1cccc(-c2cccc3c2sc2c(-c4ccccc4)cccc23)c1 ; c1ccc(-c2cccc3c2sc2c(-c4cccc(-c5cccc(-c6cnc7c8ccccc8c8ccccc8c7n6)c5)c4)cccc23)cc1]

In a 300 mL three-neck flask were put 1.04 g (2.7 mmol) of 3-(6-phenyldibenzothiophen-4-yl)phenylboronic acid, 910 mg (2.6 mmol) of 2-(3-bromophenyl)dibenzoquinoxaline, 100 mg (0.33 mmol) of tris(2-methylphenyl)phosphine, 13 mL of toluene, 3 mL of ethanol, and 4 mL of a 2M aqueous solution of potassium carbonate. After degasification by stirring, 18 mg (0.08 mmol) of palladium acetate was added to this mixture. The mixture was heated and stirred at 80° C. for 6 hours under a nitrogen stream to be reacted. After the reaction, this mixture was filtered. Toluene was added to the residue and the mixture was subjected to filtration while heated. The filtrate was concentrated to give 0.6 g of a white solid in a yield of 37.5%.

By a train sublimation method, 0.60 g of the obtained white solid was purified. In the purification, 2mDBTBPDBq-IV (abbreviation) was heated at 280° C. under a pressure of 2.8 Pa with a flow rate of an argon gas at 5.0 mL/min. Through the purification, 0.46 g of a white solid of 2mDBTBPDBq-IV (abbreviation) was obtained at a collection rate of 77%.

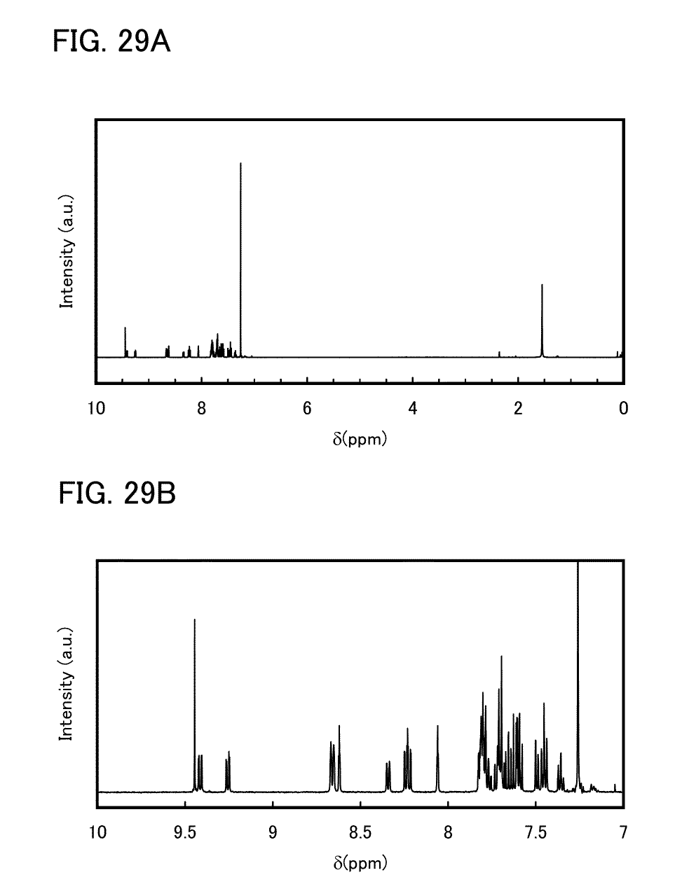

A result of nuclear magnetic resonance spectroscopy (¹H-NMR) of the compound obtained by the above synthesis method is shown below. The ¹H-NMR charts are shown in FIGS. 29A and 29B. FIG. 29B is an enlarged view of FIG. 29A within a range of 7 ppm to 10 ppm. The result reveals that 2mDBTBPDBq-IV (abbreviation), which is the heterocyclic compound of an embodiment of the present invention represented by the structural formula (131), was obtained.

¹H NMR (CDCl₃, 500 MHz): δ=7.34-7.37 (m, 1H), 7.44-7.47 (t, J=7.7 Hz, 2H), 7.49-7.50 (dd, J=7.5 Hz, 1.1 Hz, 1H), 7.58-7.73 (m, 8H), 7.75-7.83 (m, 6H), 8.06 (t, J=1.7 Hz, 1H), 8.21-8.25 (t, J=9.1 Hz, 2H), 8.33-8.35 (d, J=8.0 Hz, 1H), 8.62 (t, J=1.7 Hz, 1H), 8.65-8.67 (d, J=8.0 Hz, 2H), 9.25-9.27 (dd, J=8.0, 1.7 Hz, 1H), 9.40-9.42 (dd, J=8.0, 1.1 Hz, 1H), 9.45 (s, 1H).

Next, 2mDBTBPDBq-IV (abbreviation) obtained in this example was analyzed by liquid chromatography mass spectrometry (LC/MS).

The LC/MS was carried out with Acquity UPLC (produced by Waters Corporation) and Xevo G2 T of MS (produced by Waters Corporation).

In the MS, ionization was carried out by an electrospray ionization (ESI) method. At this time, the capillary voltage and the sample cone voltage were set to 3.0 kV and 30 V, respectively, and detection was performed in a positive mode.

A component which underwent the ionization under the above-described conditions was collided with an argon gas in a collision cell to dissociate into a plurality of product ions. Energy (collision energy) for the collision with argon was 50 eV and 70 eV. The mass range for the measurement was m/z=100 to 1200.

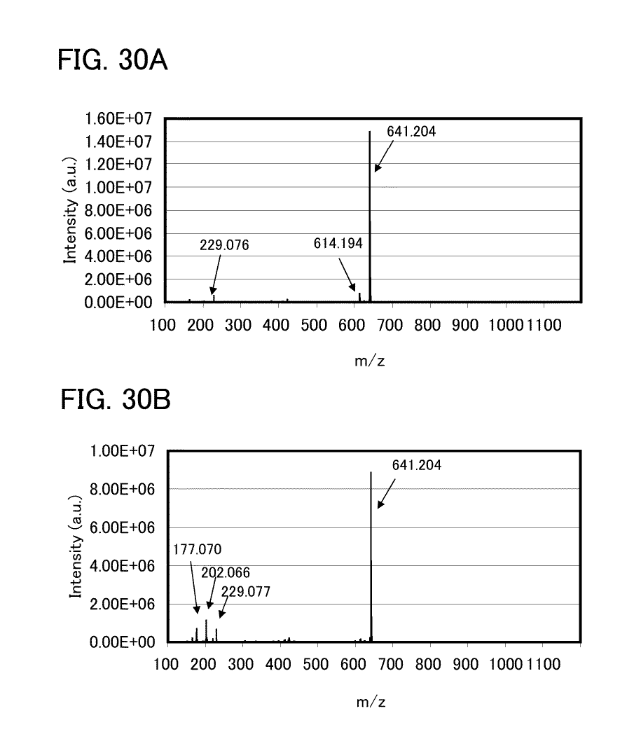

FIGS. 30A and 30B show the measurement results. Note that FIG. 30A shows the case of 50 eV and FIG. 30B shows the case of 70 eV. The result in FIG. 30B shows that product ions of 2mDBTBPDBq-IV (abbreviation), which is the heterocyclic compound of an embodiment of the present invention represented by the structural formula (131), are detected mainly around m/z=229, m/z=202, and m/z=177.

Note that the result in FIG. 30B shows characteristics derived from 2mDBTBPDBq-IV (abbreviation) and therefore can be regarded as important data for identifying 2mDBTBPDBq-IV (abbreviation) contained in the mixture.

The product ion around m/z=614 in FIG. 30A is assumed to be a cation in a state where one C atom and one N atom are detached from the dibenzo[f,h]quinoxaline ring of the compound represented by the structural formula (131), which is one feature of the heterocyclic compound of an embodiment of the present invention. Further, the product ion around m/z=229 is assumed to be a cation of a diazatriphenylenyl group such as dibenzo[f,h]quinoxaline. Moreover, the product ions around m/z=202, m/z=177, and m/z=165 are also detected at the same time. Therefore, it is indicated that 2mDBTBPDBq-IV (abbreviation) which is the heterocyclic compound of an embodiment of the present invention includes a dibenzo[f,h]quinoxaline ring.

### Example 7

### Synthesis Example 4

This example gives descriptions of a method for synthesizing 2-{3-[3-(dibenzofuran-4-yl)phenyl]phenyl}dibenzo[f,h]quinoxaline (abbreviation: 2mDBFBPDBq-II), which is the heterocyclic compound of an embodiment of the present invention represented by the structural formula (203) in Embodiment 1. A structure of 2mDBFBPDBq-II (abbreviation) is shown below.

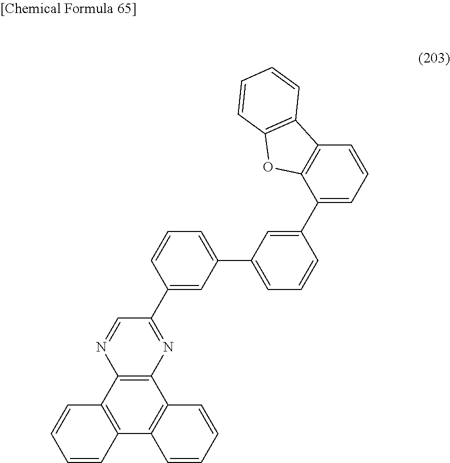 [SMILES: c1cc(-c2cccc(-c3cccc4c3oc3ccccc34)c2)cc(-c2cnc3c4ccccc4c4ccccc4c3n2)c1]

### Synthesis of 2mDBFBPDBq-II (abbreviation)

A synthesis scheme of 2mDBFBPDBq-II (abbreviation) is illustrated in (1-1).

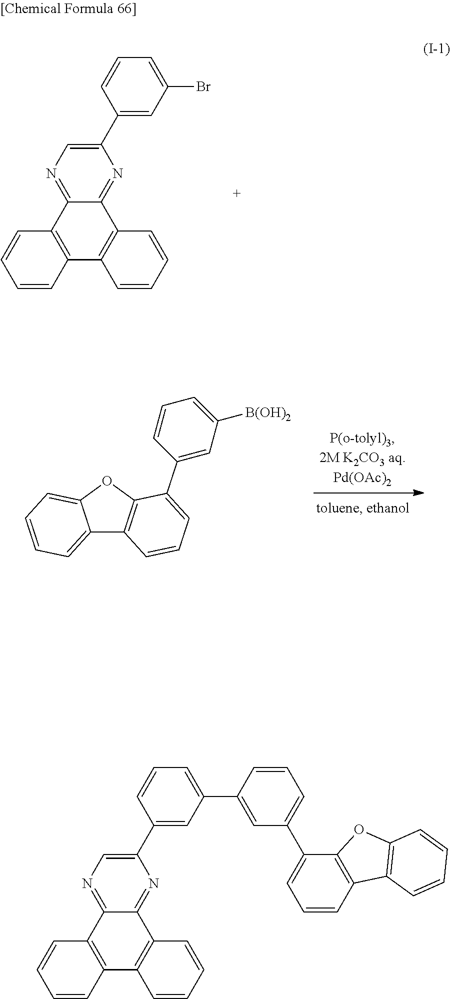 [SMILES: Brc1cccc(-c2cnc3c4ccccc4c4ccccc4c3n2)c1 ; OB(O)c1cccc(-c2cccc3c2oc2ccccc23)c1 ; c1cc(-c2cccc(-c3cccc4c3oc3ccccc34)c2)cc(-c2cnc3c4ccccc4c4ccccc4c3n2)c1]

In a 300 mL three-neck flask were put 4.0 g (10 mmol) of 2-(3-bromophenyl)dibenzo[f,h]quinoxaline, 3.3 g (11 mmol) of 3-(benzofuran-4-yl)phenylboronic acid, and 0.28 g (1.0 mmol) of tri(ortho-tolyl)phosphine, and the air in the flask was replaced with nitrogen. To this mixture were added 100 mL of toluene, 10 mL of ethanol, and 16 mL of an aqueous solution of potassium carbonate (2.0 mol/L). This mixture was degassed by stirring while the pressure was reduced. To this mixture was added 67.1 mg (0.3 mmol) of palladium(II)acetate, and the mixture was stirred at 80° C. for 1.5 hours under a nitrogen stream. After the stirring, 47.3 mg (0.2 mmol) of palladium(II)acetate was added, and stirring was performed for 2.8 hours. To this mixture were added 0.23 g (0.8 mmol) of tri(ortho-tolyl)phosphine and 91.2 mg (0.4 mmol) of palladium(II) acetate, and stirring was performed for 2.2 hours. After the stirring, the temperature of the mixture was set to 90° C., 0.23 g (0.8 mmol) of tri(ortho-tolyl)phosphine and 0.12 g (0.5 mmol) of palladium(II)acetate were added, and stirring was performed for 1 hour. After water and toluene were added to this mixture and heating was performed, this mixture was subjected to suction filtration through Celite. The aqueous layer of the obtained filtrate was extracted with toluene, and the extracted solution and the organic layer were combined and dried with magnesium sulfate. After the drying, this mixture was subjected to gravity filtration. The solid, which was obtained by concentration of the obtained filtrate, was recrystallized with a mixed solvent of toluene and ethanol to give 5.3 g of a target solid in a yield of 91%.

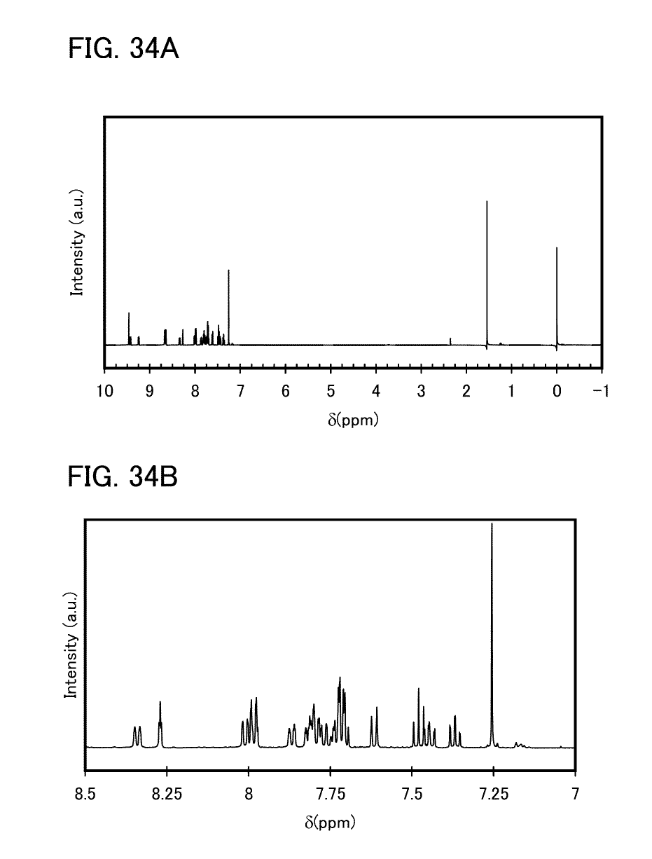

A result of nuclear magnetic resonance spectroscopy (¹H-NMR) of the compound obtained by the above synthesis method is shown below. The ¹H-NMR charts are shown in FIGS. 34A and 34B. FIG. 34B is an enlarged view of FIG. 34A within a range of 7 ppm to 8.5 ppm. The result reveals that 2mDBFBPDBq-II (abbreviation), which is the heterocyclic compound of an embodiment of the present invention represented by the structural formula (203), was obtained.

¹H NMR (CDCl₃, 500 MHz): δ=7.37 (ddd, J=7.5, 1.5 Hz, 1H), 7.43-7.50 (m, 2H), 7.62 (d, J=8.0 Hz, 1H), 7.70-7.83 (m, 8H), 7.87 (dd, J=6.0, 2.0 Hz, 1H), 7.97-8.02 (m, 3H), 8.27 (t, J=1.5 Hz, 1H), 8.34 (d, J=7.5 Hz, 1H), 8.64-8.67 (m, 3H), 9.24 (dd, J=7.5, 1.5 Hz, 1H), 9.43 (d, J=8.0 Hz, 1H), 9.46 (s, 1H).

Next, 2mDBFBPDBq-II (abbreviation) obtained in this example was analyzed by liquid chromatography mass spectrometry (LC/MS).

The LC/MS was carried out with Acquity UPLC (produced by Waters Corporation) and Xevo G2 T of MS (produced by Waters Corporation).

In the MS, ionization was carried out by an electrospray ionization (ESI) method. At this time, the capillary voltage and the sample cone voltage were set to 3.0 kV and 30 V, respectively, and detection was performed in a positive mode.

A component which underwent the ionization under the above-described conditions was collided with an argon gas in a collision cell to dissociate into a plurality of product ions. Energy (collision energy) for the collision with argon was 50 eV. The mass range for the measurement was m/z=100 to 1200.

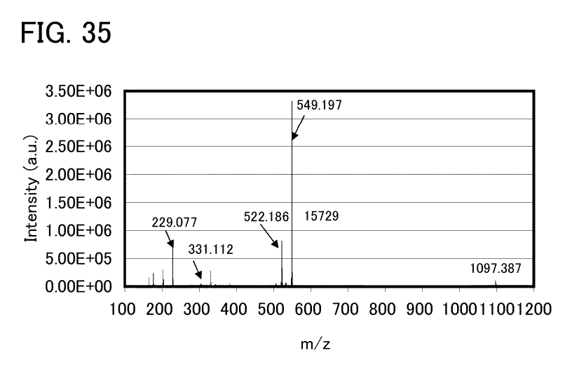

FIG. 35 shows the measurement result. The result in FIG. 35 shows that product ions of 2mDBFBPDBq-II (abbreviation), which is the heterocyclic compound of an embodiment of the present invention represented by the structural formula (203), are detected mainly around m/z=229, m/z=331, and m/z=522.

Note that the result in FIG. 35 shows characteristics derived from 2mDBFBPDBq-II (abbreviation) and therefore can be regarded as important data for identifying 2mDBFBPDBq-II (abbreviation) contained in the mixture.

The product ion around m/z=522 is assumed to be a cation in a state where one C atom and one N atom are detached from the dibenzo[f,h]quinoxaline ring of the compound represented by the structural formula (131), which is one feature of the heterocyclic compound of an embodiment of the present invention. Further, the product ion around m/z=229 is assumed to be a cation of a diazatriphenylenyl group such as dibenzo[f,h]quinoxaline. Moreover, the product ion around m/z=331 is also detected at the same time. Therefore, it is indicated that 2mDBFBPDBq-II (abbreviation) which is the heterocyclic compound of an embodiment of the present invention includes a dibenzo[f,h]quinoxaline ring.

### REFERENCE NUMERALS

101: first electrode, 102: EL layer, 103: second electrode, 111: hole-injection layer, 112: hole-transport layer, 113: light-emitting layer, 114: electron-transport layer, 115: electron-injection layer, 116: charge-generation layer, 201: anode, 202: cathode, 203: EL layer, 204: light-emitting layer, 205: phosphorescent compound, 206: first organic compound, 207: second organic compound, 301: first electrode, 302(1): first EL layer, 302(2): second EL layer, 302(n−1): (n−1)-th EL layer, 302(n): n-th EL layer, 304: second electrode, 305: charge-generation layer (I), 305(1): first charge-generation layer (I), 305(2): second charge-generation layer (I), 305(n−2): (n−2)-th charge-generation layer (I), 305(n−1): (n−1)-th charge-generation layer (I), 401: reflective electrode, 402: semi-transmissive and semi-reflective electrode, 403a: first transparent conductive layer, 403b: second transparent conductive layer, 404B: first light-emitting layer (B), 404G: second light-emitting layer (G), 404R: third light-emitting layer (R), 405: EL layer, 410R: first light-emitting element (R), 410G: second light-emitting element (G), 410B: third light-emitting element (B), 501: element substrate, 502: pixel portion, 503: driver circuit portion (source line driver circuit), 504a: driver circuit portion (gate line driver circuit), 504b: driver circuit portion (gate line driver circuit), 505: sealant, 506:

sealing substrate, 507: wiring, 508: flexible printed circuit (FPC), 509: n-channel TFT, 510: p-channel TFT, 511: switching TFT, 512: current control TFT, 513: first electrode (anode), 514: insulator, 515: EL layer, 516: second electrode (cathode), 517: light-emitting element, 518: space, 7100: television set, 7101: housing, 7103: display portion, 7105: stand, 7107: display portion, 7109: operation key, 7110: remote controller, 7201: main body, 7202: housing, 7203: display portion, 7204: keyboard, 7205: external connection port, 7206: pointing device, 7301: housing, 7302: housing, 7303: joint portion, 7304: display portion, 7305: display portion, 7306: speaker portion, 7307: recording medium insertion portion, 7308: LED lamp, 7309: operation key, 7310: connection terminal, 7311: sensor, 7312: microphone, 7400: mobile phone, 7401: housing, 7402: display portion, 7403: operation button, 7404: external connection port, 7405: speaker, 7406: microphone, 8001: lighting device, 8002: lighting device, 8003: lighting device, 8004: lighting device, 9033: clip, 9034: display-mode switching button, 9035: power button, 9036: power-saving-mode switching button, 9038: operation button, 9630: housing, 9631: display portion, 9631a: display portion, 9631b: display portion, 9632a: touch panel area, 9632b: touch panel area, 9633: solar battery, 9634: charge and discharge control circuit, 9635: battery, 9636: DCDC converter, 9637: operation key, 9638: converter, and 9639: button.

This application is based on Japanese Patent Application serial no. 2011-188235 filed with Japan Patent Office on Aug. 31, 2011, and Japanese Patent Application serial no. 2012-144180 filed with Japan Patent Office on Jun. 27, 2012, the entire contents of which are hereby incorporated by reference.

1. A light-emitting element comprising a heterocyclic compound which comprises one dibenzo[f,h]quinoxaline ring, one ring having a hole-transport skeleton, and two to eight benzene rings.

2. The light-emitting element according to claim 1, wherein a molecular weight of the heterocyclic compound is greater than or equal to 564 and less than or equal to 1000.

3. A light-emitting element comprising a heterocyclic compound which comprises one dibenzo[f,h]quinoxaline ring, one ring having a hole-transport skeleton, and four to eight benzene rings.

4. The light-emitting element according to claim 3, wherein a molecular weight of the heterocyclic compound is greater than or equal to 716 and less than or equal to 1000.

5. The light-emitting element according to claim 1, wherein the ring having the hole-transport skeleton is a carbazole ring, a dibenzothiophene ring, or a dibenzofuran ring.

6. The light-emitting element according to claim 3, wherein the ring having the hole-transport skeleton is a carbazole ring, a dibenzothiophene ring, or a dibenzofuran ring.

7. The light-emitting element according to claim 1, wherein the ring having the hole-transport skeleton is a dibenzothiophene ring.

8. The light-emitting element according to claim 3, wherein the ring having the hole-transport skeleton is a dibenzothiophene ring.

9. The light-emitting element according to claim 1, wherein the heterocyclic compound comprises a biphenyl group or a biphenyldiyl group.

10. The light-emitting element according to claim 3, wherein the heterocyclic compound comprises a biphenyl group or a biphenyldiyl group.

11. A light-emitting device comprising the light-emitting element according to claim 1.

12. A light-emitting device comprising the light-emitting element according to claim 3.

13. An electronic device comprising the light-emitting device according to claim 11.

14. An electronic device comprising the light-emitting device according to claim 12.

15. A lighting device comprising the light-emitting device according to claim 11.

16. A lighting device comprising the light-emitting device according to claim 12.

17. A heterocyclic compound having a structure represented by a general formula (G1), 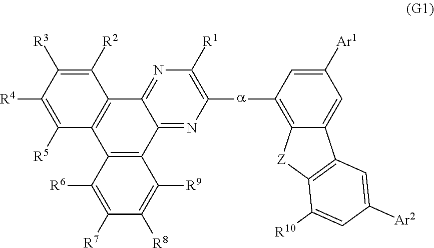
wherein in the formula:
α represents a substituted or unsubstituted phenylene group;
Ar¹ and Ar² each represent a substituted or unsubstituted biphenyl group;
R¹ to R¹⁰ independently represent hydrogen, an alkyl group having 1 to 4 carbon atoms, or a substituted or unsubstituted aryl group having 6 to 13 carbon atoms; and
Z represents oxygen or sulfur.

18. The heterocyclic compound according to claim 17, wherein the phenylene group is an m-phenylene group.

19. A heterocyclic compound having a structure represented by a general formula (G2), 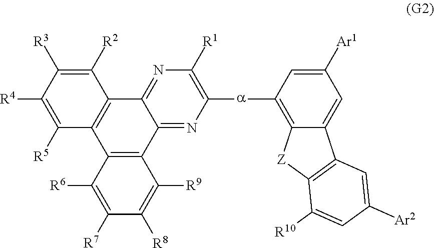
wherein in the formula:
α represents a substituted or unsubstituted biphenyldiyl group;
Ar¹ and Ar² each represent a substituted or unsubstituted phenyl group or a substituted or unsubstituted biphenyl group;
R¹ to R¹⁰ independently represent hydrogen, an alkyl group having 1 to 4 carbon atoms, or a substituted or unsubstituted aryl group having 6 to 13 carbon atoms; and
Z represents oxygen or sulfur.

20. The heterocyclic compound according to claim 19, wherein the biphenyldiyl group is a biphenyl-3,3′-diyl group.

21. The heterocyclic compound according to claim 19, wherein the heterocyclic compound is represented by a structural formula (418):

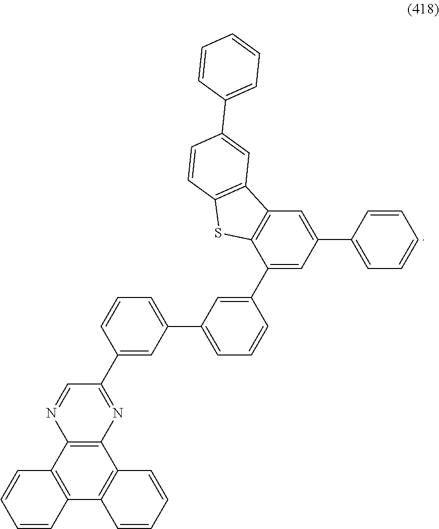 [SMILES: c1ccc(-c2ccc3sc4c(-c5cccc(-c6cccc(-c7cnc8c9ccccc9c9ccccc9c8n7)c6)c5)cc(-c5ccccc5)cc4c3c2)cc1]

22. The heterocyclic compound according to claim 17, wherein the heterocyclic compound is represented by a structural formula (400): 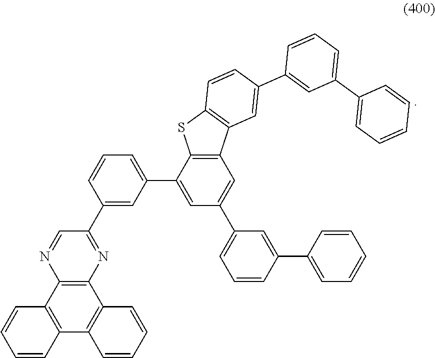 [SMILES: c1ccc(-c2cccc(-c3ccc4sc5c(-c6cccc(-c7cnc8c9ccccc9c9ccccc9c8n7)c6)cc(-c6cccc(-c7ccccc7)c6)cc5c4c3)c2)cc1]
